(19) United States
(12) Patent Application Publication
(10) Pub. No.: US 2003/0191279 A1
(43) Pub. Date: Oct. 9, 2003

(54) Urea derivatives useful as anticancer agents

(76) Inventors: Steven Wayne Goldstein, Noank, CT (US); John Adams Lowe III, Stonington, CT (US); Kelly Porter Longo, Mystic, CT (US); Mohamed Mohamed Ali Awad, Westerly, RI (US); Chakrapani Subramanyam, South Glastonbury, CT (US); Peter Hans Dorff, Norwich, CT (US)

Correspondence Address: SCULLY SCOTT MURPHY & PRESSER, PC, 400 GARDEN CITY PLAZA, GARDEN CITY, NY, 11530

(21) Appl. No.: 10/371,343

(22) Filed: Feb. 20, 2003

Related U.S. Application Data
(63) Continuation of application No. 09/649,299, filed on Aug. 28, 2000, now abandoned.
(60) Provisional application No. 60/151,161, filed on Aug. 27, 1999.

(51) Int. Cl. C07K 5/04; C07D 413/02; C 07D 4 3/02

(52) U.S. Cl. 530/331; 544/331

(57) ABSTRACT

The present invention relates to compounds of formula I

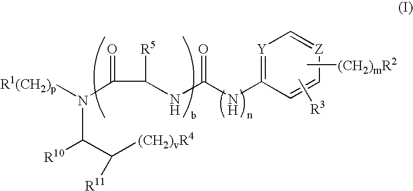

And to pharmaceutically acceptable salts, hydrates and prodrugs thereof, wherein R¹, R², R³, R⁴, R⁵, R¹⁰, R¹¹, b, m, n, p and v are as defined herein. The invention also relates to pharmaceutical compositions containing the above compounds and methods of treating hyperproliferative disorders in mammals by administering the above compounds.

### BACKGROUND OF THE INVENTION

This invention relates to novel urea derivatives that are useful in the treatment of hyperproliferative diseases, such as cancers, in mammals. This invention also relates to a method of using such compounds in the treatment of hyperproliferative diseases in mammals, especially humans, and to pharmaceutical compositions containing such compounds.

A cell may become cancerous by virtue of the transformation of a portion of its DNA into an oncogene (i.e. a gene that upon activation leads to the formation of malignant tumor cells). Many oncogenes encode proteins that are aberrant tyrosine kinases capable of causing cell transformation. Alternatively, the overexpression of a normal proto-oncogenic tyrosine kinase may also result in proliferative disorders, sometimes resulting in a malignant phenotype. It has been shown that certain tyrosine kinases may be mutated or overexpressed in many human cancers such as brain, lung, squamous cell, bladder, gastric, breast, head and neck, oesophageal, gynecological and thyroid cancers. Furthermore, the overexpression of a ligand for a tyrosine kinase receptor may result in an increase in the activation state of the receptor, resulting in proliferation of the tumor cells or endothelial cells. Thus, it is believed that the growth of mammalian cancer cells can be selectively inhibited by reducing tyrosine kinase activity.

Polypeptide growth factors, such as vascular endothelial growth factor (VEGF) having a high affinity to the human kinase insert-domain-containing receptor (KDR) or the murine fetal liver kinase 1 (FLK-1) receptor, have been associated with the proliferation of endothelial cells and more particularly vasculogenesis and angiogenesis. See PCT international application publication number WO 95/21613 (published Aug. 17, 1995). A significant body of evidence has been put forth detailing the importance of VEGF in the formation of new blood vessels (angiogenesis). It has also been noted that new blood vessel formation is crucial in supplying and maintaining the physiological conditions and nutrients necessary for tumor growth and metastasis. It has been shown that both VEGF receptor subtypes appear to be over expressed in proliferating endothelial cells located in near proximity to tumor cells in vivo. At the molecular level, intracellular portions of both FLT-1 and FLK-1 contain functional tyrosine kinase domains. Kinase activities depend on high affinity to, and interaction with, VEGF. Such interaction results in the autophosphorylation of the receptors and ultimately in endothelial cell proliferation. High affinity VEGF binding and the resulting functional effects appear to depend on the presence of specific heparin sulfate proteoglycans (VEGF glyceptor) associated with the extracellular matrix of endothelial cells. This supposition is supported by the ability of exogenous levels of heparin to inhibit VEGF induced endothelial cell proliferation by acting as a sink for secreted VEGF. By inhibiting the binding of VEGF to VEGF glyceptor (GAG), phosphorylation of tyrosine (kinase) is modulated. Agents, such as the compounds of the present invention, which are capable of modulating the KDR/FLK-1 receptor, may be used to treat disorders related to vasculogenesis or angiogenesis. Such disorders include, but are not limited to, diabetes, diabetic retinopathy, hemangioma, glioma, melanoma, Kaposi's sarcoma and ovarian, breast, lung, pancreatic, prostate, colon and epidermoid cancer.

### SUMMARY OF THE INVENTION

The present invention relates to compounds of the formula I

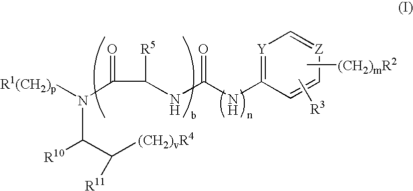

and to pharmaceutically acceptable salts, hydrates and prodrugs thereof, wherein:

R¹ and R⁵ are each independently selected from H, C₁-C₁₀ alkyl, C₂-C₁₀ alkenyl, C₂-C₁₀ alkynyl, —(CH₂)ₜ(C₆-C₁₀ aryl), or —(CH₂)ₜ(4 to 10 membered heterocyclic), wherein t is an integer from 0 to 5; said alkyl group optionally including 1 or 2 hetero moieties selected from O, S and —N(R⁶)— with the proviso that two O atoms, two S atoms, or an O and S atom are not attached directly to each other; said aryl and heterocyclic R¹ and R⁵ groups being optionally fused to a C₆-C₁₀ aryl group, a C₅-C₈ saturated cyclic group, or a 4 to 10 membered heterocyclic group; one or two carbon atoms in said 4 to 10 membered heterocyclic group of R¹ and R⁵ being optionally substituted by an oxo (═O) moiety; the ,—(CH₂)— moieties of R¹ and R⁵ optionally including a carbon-carbon double or triple bond when t is an integer from two to five; R¹ and R⁵ groups being optionally substituted by one to five R⁶ groups;

each R⁶ is independently selected from C₁-C₁₀ alkyl, C₂-C₁₀ alkenyl, C₂-C₁₀ alkynyl, halo, cyano, nitro, trifluoromethyl, trifluoromethoxy, azido, —OR⁷, —C(O)R⁸, —C(O)OR⁷, —NR⁸C(O)OR⁷, —OC(O)R⁷, —NR⁸SO₂R⁷, —SO₂NR⁷R⁸, —NR⁸C(O)R⁷, —C(O)NR¹R⁸, —NR⁷R⁸, —S(O)ⱼR⁹ wherein j is an integer ranging from zero to two, —SO₃H, —NR(CR⁸R⁹)ₜOR⁸, —(CH₂)ₜ(C₆-C₁₀ aryl), —SO₂(CH₂)ₜ(C₆-C₁₀ aryl), —S(CH₂)ₜ(C₆-C₁₀ aryl), —O(CH₂)ₜ(C₆-C₁₀ aryl), —(CH₂)ₜ(4 to 10 membered heterocyclic), and —(CR⁸R⁹)ₘOR⁸, wherein m is an integer from one to five and t is an integer from zero to five; said alkyl group optionally containing one or two hetero moieties selected from O, S and —N(R⁸)— with the proviso that two O atoms, two S atoms, or an O and S atom are not attached directly to each other; aryl and heterocyclic moieties of R⁶ being optionally fused to a C₆-C₁₀ aryl group, a C₅-C₈ saturated cyclic group, or a 4 to 10 membered heterocyclic group; one or two carbon atoms of the heterocyclic moieties. of R⁶ being optionally substituted by an oxo (═O) moiety; and the alkyl, aryl and heterocyclic moieties of R⁶ groups being optionally substituted by one to three substituents independently selected from halo, cyano, nitro, trifluoromethyl, trifluoromethoxy, azido, —NR⁸SO₂R⁷, —SO₂NR⁷R⁸, —C(O)R⁷, —C(O)OR⁷, —OC(O)R⁷, —NR⁸C(O)R⁷, —C(O)NR⁷R⁸, —NR⁷R⁸, —(CR⁸R⁹)ₘOR⁸ wherein m is an integer from one to five, —OR⁷ and R⁷;

each R⁷ is independently selected from H, C₁-C₁₀ alkyl, —(CH₂)ₜ(C₆-C₁₀ aryl), and —(CH₂)ₜ(4 to 10 membered heterocyclic), wherein t is an integer from zero to five; said alkyl group optionally including one or two hetero moieties selected from O, S and —N(R⁶)— with the proviso that two O atoms, two S atoms, or an O and S atom are not attached directly to each other; said aryl and heterocyclic R⁷ groups being optionally fused to a C₆-C₁₀ aryl group, a C₅-C₈ saturated cyclic group, or a 4 to 10 membered heterocyclic group; the foregoing moieties of R⁷, with the exception of H, being optionally substituted by one to three substituents independently selected from halo, cyano, nitro, trifluoromethyl, trifluoromethoxy, azido, —C(O)R⁸, —C(O)OR⁸, —CO(O)R⁸, —NR⁸C(O)R⁹, —C(O)NR⁸R⁹, —NR⁸R⁹, hydroxy, C₁-C₆ alkyl, and C₁-C₆ alkoxy;

each R⁸ and R⁹ is independently H or C₁-C₆ alkyl;

R² is a group having an acidic proton, for example, CO₂H, CONHSO₂R¹, CONR¹(CH₂)CO₂H, SO₂H, PO₃H₂,

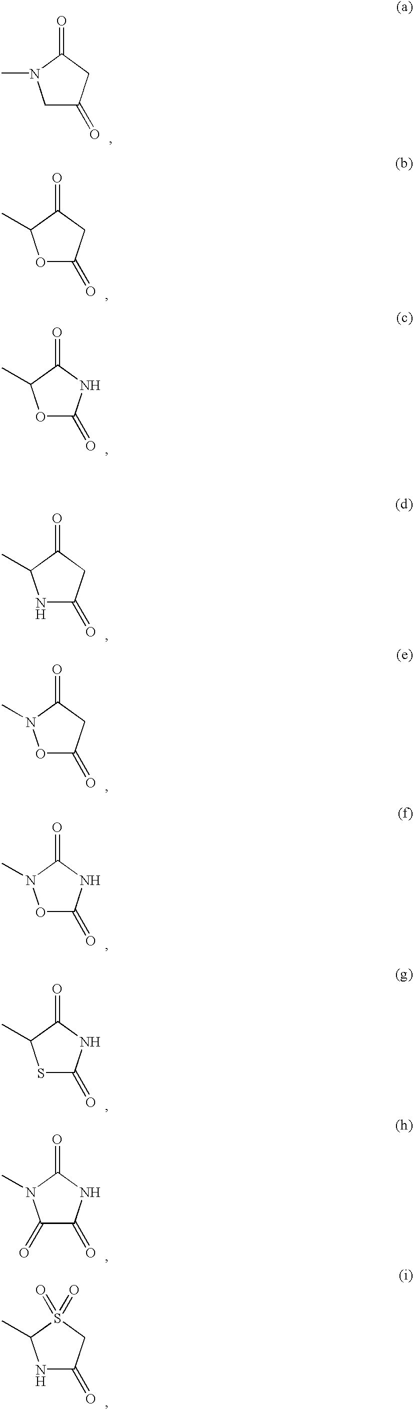

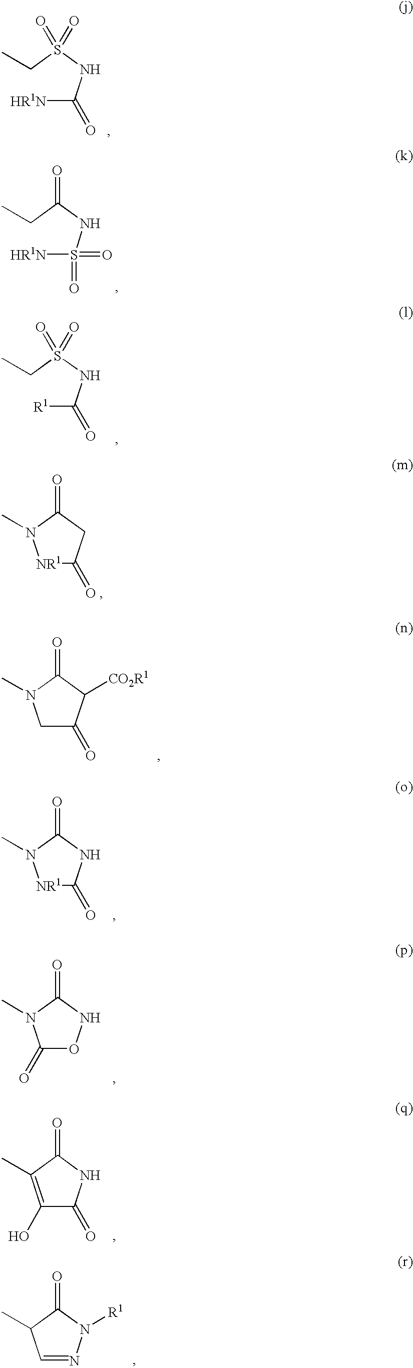

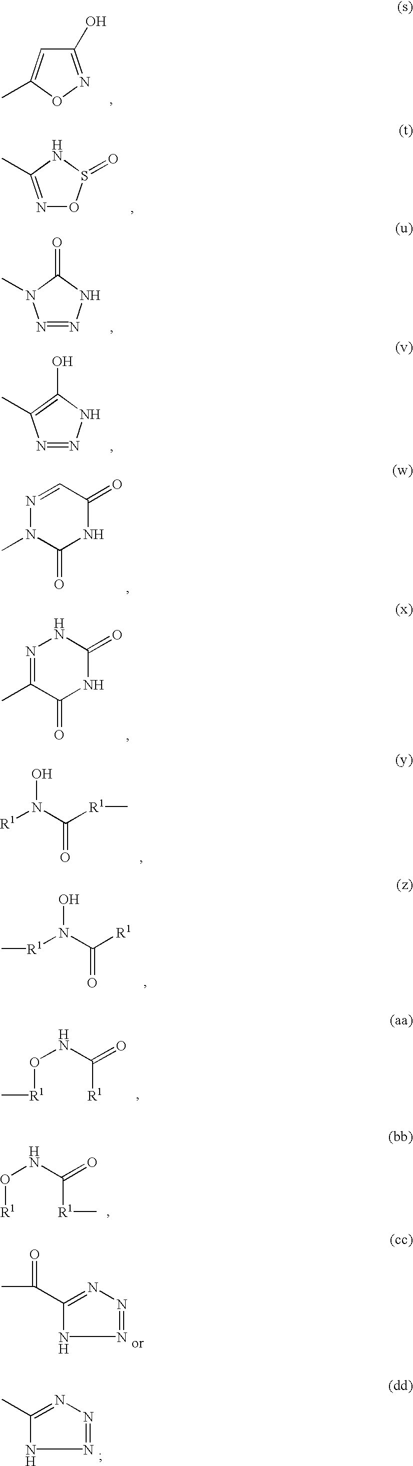

each R³ is independently selected from H and R²;

R⁴ is —(CH₂)ₜ(C₆-C₁₀ aryl), or —(CH₂)ₜ(4 to 10 membered heterocyclic), wherein t is an integer from zero to five; said alkyl group optionally including one or two hetero moieties selected from O, S and —N(R⁶)— with the proviso that two O atoms, two S atoms, or an O and S atom are not attached directly to each other; said aryl and heterocyclic R⁴ groups are optionally fused to a C₆-C₁₀ aryl group, a C₅-C₈ saturated cyclic group, or a 4 to 10 membered heterocyclic group; one or two carbon atoms of the heterocyclic moieties of R⁴ being optionally substituted by an oxo (═O) moiety; the —(CH₂)ₜ— moieties of R⁴ optionally including a carbon-carbon double or triple bond where t is an integer from two to five, R⁴ being optionally substituted by one to five R⁶ groups or methylenedioxy;

R¹⁰ and R¹¹ are each independently R¹, or R¹⁰ and R¹¹, together with the carbons to which R¹⁰ and R¹¹ are attached, optionally form a 4 to 10 membered carbocyclic group optionally substituted by ═O or H(OH) or a 4 to 10 membered heterocyclic group comprising heterocyclic moieties selected from O, N or S optionally substituted with R¹, S, SO or SO₂; said carbocyclic group or heterocyclic group formed by R¹⁰ and R¹¹ being optionally fused to a C₆-C₁₀ aryl group, a C₅-C₈ saturated cyclic group, or a 4 to 10 membered heterocyclic group optionally substituted with one or more substituents selected from halogen, hydroxy, C₁-C₁₀ alkyl, C₁-C₁₀ alkoxy and methylenedioxy;

Y and Z are independently CH, N optionally substituted with R¹, O, S, SO or SO₂;

m is zero or 1;

n is zero or 1;

b is zero or 1;

v is zero or 1 and

p is zero to 6,

with the proviso that said compound of formula I is not 3-(3-{[benzyl-(2-benzyl-1,2,3,4-tetrahydro-naphthalen-1-yl)-carbamoyl]-methyl}-ureido)-benzoic acid or 3-(3-{[2(2-benzyl-1,2,3,4-tetrahydro-naphthalen-1-yl)-isoxalol-5-ylmethyl-carbamoyl]-methyl}-ureido)-benzoic acid.

Preferred compounds include those of formula I wherein R¹ and R⁵ are each independently selected from H, C₁-C₁₀ alkyl, C₂-C₁₀ alkenyl, C₂-C₁₀ alkynyl, C₆-C₁₀ aryl, or a 4 to 10 membered heterocyclic group, wherein any. aromatic carbocyclic or heterocyclic rings are optionally substituted with one or more substituents selected from halogen, hydroxy, C₁-C₆alkoxy, C₁-C₆alkyl, CF₃, CO₂H, CO₂-C₁-C₆alkyl or CN.

Other preferred compounds are those in which R² is —CO₂H, —CONHSO₂R¹, —CONR¹(CH₂)CO₂H,

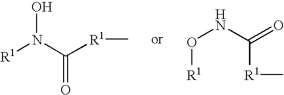

Other preferred compounds include those wherein R₂ is meta-substituted benzoic acid or phenylacetic acetic acid.

Other preferred compounds are those wherein R⁴ is phenyl or a 4 to 10 membered heterocyclic group optionally substituted with one or more substituents selected from halogen, hydroxy, C₁-C₆alkoxy, C₁-C₆alkyl, or methylenedioxy.

Other preferred compounds are those wherein Y and Z are independently selected from CH and N.

Another preferred class of compounds comprises compounds of the formula

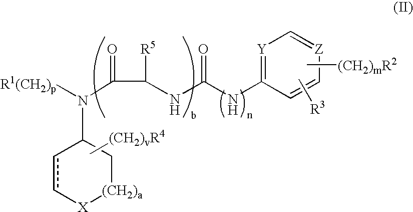

wherein R¹, R², R³, R⁴, R⁵, b, m, n, p, v, Y and Z are as defined for formula I, X is CHR¹, O, NR₁, S, SO or SO₂, a is zero, 1 or 2; and the dotted line indicates optional fusion to a C₆-C₁₀ aryl group, a C₅-C₈ saturated cyclic group, or a 4 to 10 membered heterocyclic group, each optionally substituted with one or more substituents selected from halogen, hydroxy, C₁-C₁₀ alkyl, C₁-C₁₀ alkoxy and methylenedioxy.

Specific preferred compounds of the present invention include:

trans-3-(3-{[Benzyl-(2-benzyl-1,2,3,4-tetrahydro-naphthalen-1-yl)-carbamoyl]-methyl}-ureido)-benzoic acid;

trans-2-[3-(3-Benzenesulfonylaminocarbonyl-phenyl)-ureido]-N-benzyl-N-(2-benzyl-1,2,3,4-tetrahydro-naphthalen-1-yl)-acetamide;

trans-3-(3-{[Benzyl-(2-benzyl-1,2,3,4-tetrahydro-naphthalen-1-yl)-carbamoyl]-methyl}-ureido)-N-(1H-tetrazol-5-yl)-benzamide;

trans-N-{[Benzyl-(2-benzyl-1,2,3,4-tetrahydro-naphthalen-1-yl)-carbamoyl]-methyl}-3-(1H-tetrazol-5-yl)-benzamide;

trans-[3-(3-{[Benzyl-(2-benzyl-1,2,3,4-tetrahydro-naphthalen-1-yl)-carbamoyl]-methyl}-ureido)-phenyl]-acetic acid;

cis-[3-(3-{[Benzyl-(2-benzyl-1,2,3,4-tetrahydro-naphthalen-1-yl)-carbamoyl]-methyl}-ureido)-phenyl]-acetic acid;

cis-3-(3-{[(2-Benzyl-1,2,3,4-tetrahydro-naphthalen-1-yl)-furan-2-ylmethyl-carbamoyl]-methyl}-ureido)-benzoic acid;

trans-3-(3-{[(2-Benzyl-1,2,3,4-tetrahydro-naphthalen-1-yl)-furan-2-ylmethyl-carbamoyl]-methyl}-ureido)-benzoic acid;

trans-3-(3-{[(2-Benzyl-1,2,3,4-tetrahydro-naphthalen-1-yl)-isoxazol-5-ylmethyl-carbamoyl]-methyl}-ureido)-benzoic acid;

trans-3-(3-{[(2-Benzyl-1,2,3,4-tetrahydro-naphthalen-1-yl)-(4-fluoro-benzyl)-carbamoyl]-methyl}-ureido)-benzoic acid;

trans-3-(3-{[(2-Benzyl-1,2,3,4-tetrahydro-naphthalen-1-yl)-(2-methoxy-benzyl)-carbamoyl]-methyl}-ureido)-benzoic acid;

trans-3-(3-{[(2-Benzyl-1,2,3,4-tetrahydro-naphthalen-1-yl)-pyridin-2-ylmethyl-carbamoyl]-methyl}-ureido)-benzoic acid;

trans-3-(3-{[(2-Benzyl-1,2,3,4-tetrahydro-naphthalen-1-yl)-thiophen-2-ylmethyl-carbamoyl]-methyl}-ureido)-benzoic acid;

trans-3-(3-{[Benzyl-(2-benzyl-cyclohexyl)-carbamoyl]-methyl}-ureido)-benzoic acid

trans-3-(3-{[(2-Benzyl-1,2,3,4-tetrahydro-naphthalen-1-yl)-pyridin-4-ylmethyl-carbamoyl]-methyl}-ureido)-benzoic acid;

trans-3-(3-{[(2-Benzyl-1,2,3,4-tetrahydro-naphthalen-1-yl)-ethyl-carbamoyl]-methyl}-ureido)-benzoic acid;

trans-3-(3-{[(2-Benzyl-1,2,3,4-tetrahydro-naphthalen-1-yl)-thiazol-2-ylmethyl-carbamoyl]-methyl}-ureido)-benzoic acid;

trans-3-[3-({Benzyl-[2-(2-methoxy-benzyl)-1,2,3,4-tetrahydro-naphthalen-1-yl]-carbamoyl}-methyl)-ureido]-benzoic acid;

trans-3-[3-({Benzyl-[2-(2-fluoro-benzyl)-1,2,3,4-tetrahydro-naphthalen-1-yl]-carbamoyl}-methyl)-ureido]-benzoic acid;

trans-6-({[Benzyl-(2-benzyl-cyclohexyl)-carbamoyl]-methyl}-carbamoyl)-pyrimidine-4-carboxylic acid;

trans-4-(3-{1-[Benzyl-(2-benzyl-1,2,3,4-tetrahydro-naphthalen-1-yl)-carbamoyl]-2-phenyl-ethyl}-ureido)-phthalic acid;

trans-4-[3-Benzyl-3-(2-benzyl-1,2,3,4-tetrahydro-naphthalen-1-yl)-ureido]-phthalic acid;

3-(3-{[Benzyl-(3-benzyl-chroman-4-yl)-carbamoyl]-methyl}-ureido)-benzoic acid;

3-(3-{[Benzyl-(2-benzyl-7-methoxy-1,2,3,4-tetrahydro-naphthalen-1-yl)-carbamoyl]-methyl}-ureido)-benzoic acid;

3-(3-{[Benzyl-(1,3-diphenyl-propyl)-carbamoyl]-methyl}-ureido)-benzoic acid;

3-(3-{[Benzyl-(2-benzyl-indan-1-yl)-carbamoyl]-methyl}-ureido)-benzoic acid;

3-(3-{[Benzyl-(2-benzyl-5,7-dimethyl-1,2,3,4-tetrahydro-naphthalen-1-yl)-carbamoyl]-methyl}-ureido)-benzoic acid;

3-(3-{[Benzyl-(2-benzyl-6,7-dimethoxy-1,2,3,4-tetrahydro-naphthalen-1-yl)-carbamoyl]-methyl}-ureido)-benzoic acid;

3-[3-({Benzyl-[2-(4-methyl-benzyl)-1,2,3,4-tetrahydro-naphthalen-1-yl]-carbamoyl}-methyl)-ureido]-benzoic acid;

3-[3-({Benzyl-[2-(3-methyl-benzyl)-1,2,3,4-tetrahydro-naphthalen-1-yl]-carbamoyl}-methyl)-ureido]-benzoic acid;

3-[3-({Benzyl-[2-(3-methoxy-benzyl)-1,2,3,4-tetrahydro-naphthalen-1-yl]-carbamoyl}-methyl)-ureido]-benzoic acid;

3-[3-({Benzyl-[2-(3,4-dichloro-benzyl)-1,2,3,4-tetrahydro-naphthalen-1-yl]-carbamoyl}-methyl)-ureido]-benzoic acid;

3-[3-({Benzyl-[2-(2-methyl-benzyl)-1,2,3,4-tetrahydro-naphthalen-1-yl]-carbamoyl}-methyl)-ureido]-benzoic acid;

3-[3-({Benzyl-[2-(3-fluoro-benzyl)-1,2,3,4-tetrahydro-naphthalen-1-yl]-carbamoyl}-methyl)-ureido]-benzoic acid;

3-(3-{[(2-Benzo[1,3]dioxol-5-ylmethyl-1,2,3,4-tetrahydro-naphthalen-1-yl)-benzyl-carbamoyl]-methyl}-ureido)-benzoic acid;

3-[3-({Benzyl-[2-(2-chloro-benzyl)-1,2,3,4-tetrahydro-naphthalen-1-yl]-carbamoyl}-methyl)-ureido]-benzoic acid;

3-[3-({Benzyl-[2-(4-fluoro-benzyl)-1,2,3,4-tetrahydro-naphthalen-1-yl]-carbamoyl}-methyl)-ureido]-benzoic acid;

3-[3-({Benzyl-[2-(4-methoxy-benzyl)-1,2,3,4-tetrahydro-naphthalen-1-yl]-carbamoyl}-methyl)-ureido]-benzoic acid;

3-[3-({Benzyl-[2-(4-chloro-benzyl)-1,2,3,4-tetrahydro-naphthalen-1-yl]-carbamoyl}-methyl)-ureido]-benzoic acid;

3-(3-{[Benzyl-(2-benzyl-6-methoxy-1,2,3,4-tetrahydro-naphthalen-1-yl)-carbamoyl]-methyl}-ureido)-benzoic acid;

3-(3-{[2-benzyl-1,2,3,4-tetrahydro-naphthalen-1-yl)-furan-2-ylmethyl-carbamoyl]-methyl}-ureido)-benzoic acid;

N-{[benzyl-(2-benzyl-1,2,3,4-tetrahydro-napthalen-1-yl)-carbamoyl]-methyl}-3-(1H-tetrazol-5-yl)-benzamide; and

2-[3-(3-benzenesulfonylaminocarbonyl-phenyl)-ureido]-N-benzyl-N-(2-benzyl-1,2,3,4-tetrahydro-naphthalen-1-yl)-acetamide.

Particularly preferred compounds of the present invention include:

3-[3-({Benzyl-[2-(3-fluoro-benzyl)-1,2,3,4-tetrahydro-naphthalen-1-yl]-carbamoyl}-methyl)-ureido]-benzoic acid;

3-(3-{[(2-Benzo[1,3]dioxol-5-ylmethyl-1,2,3,4-tetrahydro-naphthalen-1-yl)-benzyl-carbamoyl]-methyl}-ureido)-benzoic acid;

3-[3-({Benzyl-[2-(2-chloro-benzyl)-1,2,3,4-tetrahydro-naphthalen-1-yl]-carbamoyl}-methyl)-ureido]-benzoic acid;

3-[3-({Benzyl-[2-(4-fluoro-benzyl)-1,2,3,4-tetrahydro-naphthalen-1-yl]-carbamoyl}-methyl)-ureido]-benzoic acid;

3-[3-({Benzyl-[2-(4-methoxy-benzyl)-1,2,3,4-tetrahydro-naphthalen-1-yl]-carbamoyl}-methyl)-ureido]-benzoic acid;

3-[3-({Benzyl-[2-(4-chloro-benzyl)-1,2,3,4-tetrahydro-naphthalen-1-yl]-carbamoyl}-methyl)-ureido]-benzoic acid; and

3-(3-{[Benzyl-(2-benzyl-6-methoxy-1,2,3,4-tetrahydro-naphthalen-1-yl)-carbamoyl]-methyl}-ureido)-benzoic acid.

3-(3-{[2-benzyl-1,2,3,4-tetrahydro-naphthalen-1-yl)-furan-2-ylmethyl-carbamoyl]-methyl}-ureido)-benzoic acid;

N-{[benzyl-(2-benzyl-1,2,3,4-tetrahydro-napthalen-1-yl)-carbamoyl]-methyl}-3-(1H-tetrazol-5-yl)-benzamide; and

2-[3-(3-benzenesulfonylaminocarbonyl-phenyl)-ureido]-N-benzyl-N-(2-benzyl-1,2,3,4-tetrahydro-naphthalen-1-yl)-acetamide.

The invention also relates to a pharmaceutical composition for the treatment of a hyperproliferative disorder in a mammal which comprises a therapeutically effective amount of a compound of formula I, or a pharmaceutically acceptable salt or hydrate thereof, and a pharmaceutically acceptable carrier. In one embodiment, said pharmaceutical composition is for the treatment of cancer such as. brain, lung, squamous cell, bladder, gastric, pancreatic, breast, head, neck, renal, prostate, colorectal, oesophageal, gynecological (such as ovarian) or thyroid cancer. In another embodiment, said pharmaceutical composition is for the treatment of a non-cancerous hyperproliferative disorder such as benign hyperplasia of the skin (e.g., psoriasis) or prostate (e.g., benign prostatic hypertropy (BPH)).

The invention also relates to a pharmaceutical composition for the treatment of pancreatitis or kidney disease (including proliferative glomerulonephritis and diabetes-induced renal disease) in a mammal which comprises a therapeutically effective amount of a compound of formula I, or a pharmaceutically acceptable salt or hydrate thereof, and a pharmaceutically acceptable carrier.

The invention also relates to a pharmaceutical composition for the prevention of blastocyte implantation in a mammal which comprises a therapeutically effective amount of a compound of formula I, or a pharmaceutically acceptable salt or hydrate thereof, and a pharmaceutically acceptable carrier.

The invention also relates to a pharmaceutical composition for treating a disease related to vasculogenesis or angiogenesis in a mammal which comprises a therapeutically effective amount of a compound of formula I, or a pharmaceutically acceptable salt or hydrate thereof, and a pharmaceutically acceptable carrier. In one embodiment, said pharmaceutical composition is for treating a disease selected from the group consisting of tumor angiogenesis, chronic inflammatory disease such as rheumatoid arthritis, atherosclerosis, skin diseases such as psoriasis, excema, and scleroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

The invention also relates to a method of treating a hyperproliferative disorder in a mammal which comprises administering to said mammal a therapeutically effective amount of the compound of formula I, or 3-(3-{[benzyl-(2-benzyl-1,2,3,4-tetrahydro-naphthalen-1-yl)-carbamoyl]-methyl}-ureido)-benzoic acid or 3-(3-{[2(2-benzyl-1,2,3,4-tetrahydro-naphthalen-1-yl)-isoxalol-5-ylmethyl-carbamoyl]-methyl}-ureido)-benzoic acid, or a pharmaceutically acceptable salt or hydrate thereof. In one embodiment, said method relates to the treatment of cancer such as brain, squamous cell, bladder, gastric, pancreatic, breast, head, neck, oesophageal, prostate, colorectal, lung, renal, gynecological (such as ovarian) or thyroid cancer. In another embodiment, said method relates to the treatment of a non-cancerous hyperproliferative disorder such as benign hyperplasia of the skin (e.g., psoriasis) or prostate (e.g., benign prostatic hypertropy (BPH)).

The invention also relates to a method for the treatment of a hyperproliferative disorder in a mammal which comprises administering to said mammal a therapeutically effective amount of a compound of formula I, or 3-(3-{[benzyl-(2-benzyl-1,2,3,4-tetrahydro-naphthalen-1-yl)-carbamoyl]-methyl)-ureido)-benzoic acid or 3-(3-{[2(2-benzyl-1,2,3,4-tetrahydro-naphthalen-1-yl)-isoxalol-5-ylmethyl-carbamoyl]-methyl}-ureido)-benzoic acid or a pharmaceutically acceptable salt or hydrate thereof, in combination with an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, and anti-androgens.

The invention also relates to a method of treating pancreatitis or kidney disease in a mammal which comprises administering to said mammal a therapeutically effective amount of a compound of formula I, or 3-(3-{[benzyl-(2-benzyl-1,2,3,4-tetrahydro-naphthalen-1-yl)-carbamoyl]-methyl)-ureido)-benzoic acid or 3-(3-{[2(2-benzyl-1,2,3,4-tetrahydro-naphthalen-1-yl)-isoxalol-5-ylmethyl-carbamoyl]-methyl}-ureido)-benzoic acid, or a pharmaceutically: acceptable salt or hydrate thereof.

The invention also relates to a method of preventing blastocyte implantation in a mammal which comprises administering to said mammal a therapeutically effective amount of a compound of formula I, or 3-(3-([benzyl-(2-benzyl-1,2,3,4-tetrahydro-naphthalen-1-yl)-carbamoyl]-methyl}-ureido)-benzoic acid or 3-(3-{[2(2-benzyl-1,2,3,4-tetrahydro-naphthalen-1-yl)-isoxalol-5-ylmethyl-carbamoyl]-methyl}-ureido)-benzoic acid, or a pharmaceutically acceptable salt or hydrate thereof.

The invention also relates to a method of treating diseases related to vasculogenesis or angiogenesis in a mammal which comprises administering to said mammal an effective amount of a compound of formula I, or 3-(3-{[benzyl-(2-benzyl-1,2,3,4-tetrahydro-naphthalen-1-yl)-carbamoyl]-methyl}-ureido)-benzoic acid or 3-(3-{[2(2-benzyl-1,2,3,4-tetrahydro-naphthalen-1-yl)-isoxalol-5-ylmethyl-carbamoyl]-methyl}-ureido)-benzoic acid, or a pharmaceutically acceptable salt or hydrate thereof. In one embodiment, said method is for treating a disease selected from the group consisting of tumor angiogenesis, chronic inflammatory disease such as rheumatoid arthritis, atherosclerosis, skin diseases such as psoriasis, excema, and scieroderma, diabetes, diabetic retinopathy, retinopathy of prematurity, age-related macular degeneration, hemangioma, glioma, melanoma, Kaposi's sarcoma and ovarian, breast, lung, pancreatic, prostate, colon and epidermoid cancer.

Further, the compounds of the present-invention, as well as 3-(3-{[benzyl-(2-benzyl-1,2,3,4-tetrahydro-naphthalen-1-yl )-carbamoyl]-methyl}-ureido)-benzoic acid or 3-(3-([2(2-benzyl-1,2,3,4-tetrahydro-naphthalen-1-yl)-isoxalol-5-ylmethyl-carbamoyl]-methyl}-ureido)-benzoic acid, may be used as contraceptives in mammals.

Patients that can be treated with the compounds of formula I, or 3-(3-{[benzyl-(2-benzyl-1,2,3,4-tetrahydro-naphthalen-1-yl)-carbamoyl]-methyl}-ureido)-benzoic acid or 3-(3-{[2(2-benzyl-1,2,3,4-tetrahydro-naphthalen-1-yl)-isoxalol-5-ylmethyl-carbamoyl]-methyl}-ureido)-benzoic acid, and the pharmaceutically acceptable salts and hydrates of said compounds, according to the methods of this invention include, for example, patients that have been diagnosed as having psoriasis, BPH, lung cancer, bone cancer, pancreatic cancer, skin cancer, cancer of the head and neck, cutaneous or intraocular melanoma, uterine cancer, ovarian cancer, rectal cancer or cancer of the anal region, stomach cancer, colon cancer, breast cancer, gynecologic tumors (e.g., uterine sarcomas, carcinoma of the fallopian tubes, carcinoma of the endometrium, carcinoma of the cervix, carcinoma of the vagina or carcinoma of the vulva), Hodgkin's disease, cancer of the esophagus,,cancer of the small intestine, cancer of the endocrine system (e.g., cancer of the thyroid, parathyroid or adrenal glands), sarcomas of soft tissues, cancer of the urethra, cancer of the penis, prostate cancer, chronic or acute leukemia, solid tumors of childhood, lymphocytic, lymphonas, cancer of the bladder, cancer of the kidney or ureter (e.g., renal cell carcinoma, carcinoma of the renal pelvis), or neoplasms of the central nervous system (e.g., primary CNS lymphoma, spinal axis tumors, brain stem gliomas or pituitary adenomas).

The invention is further directed to a process for forming a compound of formula III

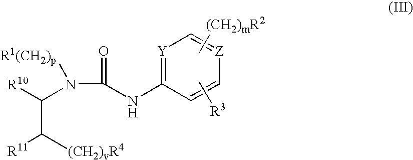

which comprises reacting a compound of formula VIII

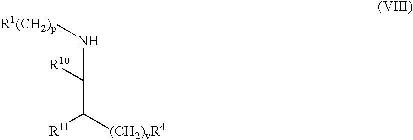

with a compound of formula IX

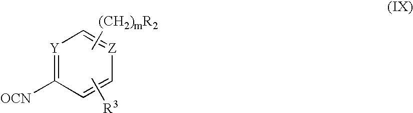

wherein R¹, R², R³, R⁴, R¹⁰, R¹¹, m, p and v are as defined for formula I.

The invention also relates to a process for forming a compound of formula IV

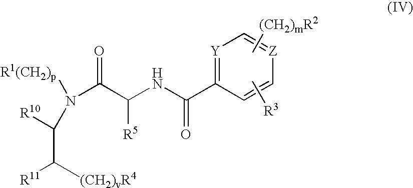

which comprises reacting a compound of formula XII

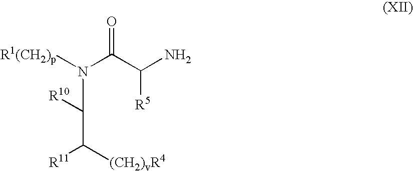

with a compound of formula XIII

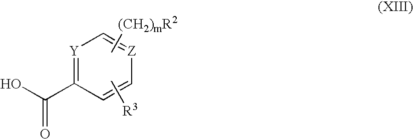

wherein R¹, R², R³, R⁴, R⁵, R¹⁰, R¹¹, m, p, and v are as defined for formula I.

The invention is still further directed to a method of forming a compound of formula V

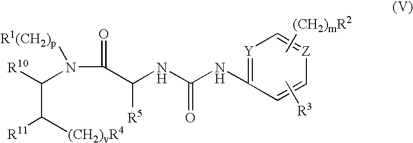

which comprises reacting a compound of formula XII

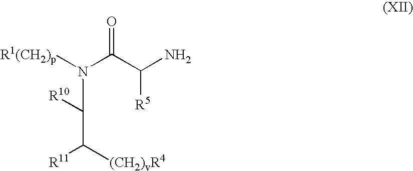

with a compound of formula IX

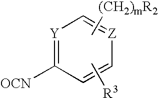

wherein R¹, R², R³, R⁴, R⁵, R¹⁰, R¹¹, m, p and v are as defined for formula I.

The term “halo”, as used herein, unless otherwise indicated, includes fluoro, chloro, bromo or iodo. Preferred halo groups are fluoro, chloro and bromo.

The term “alkyl”, as used herein, unless otherwise indicated, includes saturated monovalent hydrocarbon radicals having straight, cyclic or branched moieties. It is understood that for cyclic moieties at least three carbon atoms are required in said alkyl group.

The term “alkenyl”, as used herein, unless otherwise indicated, includes monovalent hydrocarbon radicals having at least one carbon-carbon double bond and also having straight, cyclic or branched moieties as provided above in the definition of “alkyl”.

The term “alkynyl”, as used herein, unless otherwise indicated, includes monovalent hydrocarbon radicals having at least one carbon-carbon triple bond and also having straight, cyclic or branched moieties as provided above in the definition of “alkyl”.

The term “alkoxy”, as used herein, unless otherwise indicated, includes O-alkyl groups wherein “alkyl” is as defined above.

The term “aryl”, as used herein, unless otherwise indicated, includes an organic radical derived from an aromatic hydrocarbon by removal of one hydrogen, such as phenyl or naphthyl.

The term “4-10 membered heterocyclic”, as used herein, unless otherwise indicated, includes aromatic and non-aromatic heterocyclic groups containing one or more heteroatoms each selected from O, S and N, wherein each heterocyclic group has from 4-10 atoms in its ring system. Non-aromatic heterocyclic groups include groups having only 4 atoms in their ring system, but aromatic heterocyclic groups must have at least 5 atoms in their ring system. An example of a 4 membered heterocyclic group is azetidinyl (derived from azetidine). An example of a 5 membered heterocyclic group is thiazolyl and an example of a 10 membered heterocyclic group is quinolinyl. Examples of non-aromatic heterocyclic groups are pyrrolidinyl, tetrahydrofuranyl, tetrahydrothienyl, tetrahydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the compounds listed above, may be C-attached, S-attached or N-attached where such is possible. For instance, a group derived from pyrrole may be pyrrol-1-yl (N-attached) or pyrrol-3-yl (C-attached).

The phrase “pharmaceutically acceptable salt(s)”, as used herein, unless otherwise indicated, includes salts of acidic or basic groups which may be present in the compounds of formula I. The compounds of, formula I that are basic in nature are capable of forming a wide variety of salts with various inorganic and organic acids. The acids that may be used to prepare pharmaceutically acceptable acid addition salts of such basic compounds of formula I are those that form non-toxic acid addition salts, i.e., salts containing pharmacologically acceptable anions, such as the hydrochloride, hydrobromide, hydroiodide, nitrate, sulfate, bisulfate, phosphate, acid phosphate, isonicotinate, acetate, lactate, salicylate, citrate, acid citrate, tartrate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate [i.e., 1,1′-methylene-bis-(2-hydroxy-3-naphthoate)] salts.

Those compounds of the formula I that are acidic in nature are capable of forming base salts with various pharmacologically acceptable cations. Examples of such salts include the alkali metal or alkaline earth metal salts and particularly, the sodium and potassium salts.

Certain compounds of formula I may have asymmetric centers and therefore exist in different enantiomeric forms. This invention relates to the use of all optical isomers and stereoisomers of the compounds of formula I and mixtures thereof. The compounds of formula I may also exist as tautomers. This invention relates to the use of all such tautomers and mixtures thereof.

The subject invention also includes isotopically-labelled compounds, and the pharmaceutically acceptable salts thereof, which are identical to those recited in formula I, but for the fact that one or more atoms are replaced by an atom having anatomic mass or mass number different from the atomic mass or mass number usually found in nature. Examples of isotopes that can be incorporated into compounds of the invention include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³⁵S, ¹⁸F, and ³⁶Cl, respectively. Compounds, of the present invention, prodrugs thereof, and pharmaceutically acceptable salts of said compounds or of said prodrugs which contain the aforementioned isotopes and/or other isotopes of other atoms are within the scope of this invention. Certain isotopically-labelled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labelled compounds of formula I of this invention and prodrugs thereof can generally be prepared by carrying out the procedures disclosed in the Schemes and/or in the Examples and Preparations below, by substituting a readily available isotopically labelled reagent for a non-isotopically labelled reagent.

This invention also encompasses pharmaceutical compositions containing, and methods of treating diseases related to vasculogenesis or angiogenesis in mammals through administration of prodrugs of, compounds of the formula I. Compounds of formula I having free amino, amido, hydroxy or carboxylic groups can be converted into prodrugs. Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of formula I. The amino acid residues include, but are not limited to, the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline homocysteine, homoserine, ornithine and methionine sulfone.

Additional types of prodrugs are also encompassed. For instance, free carboxyl groups can be derivatized as amides or alkyl esters. The amide and ester moieties may incorporate groups including, but not limited, to ether, amine and carboxylic acid functionalities. Free hydroxy groups may be derivatized using groups including, but not limited to, hemisuccinates, phosphate esters, dimethylaminoacetates, and phosphoryloxymethyloxycarbonyls, as outlined in D. Fleisher, R. Bong, B. H. Stewart, Advanced Drug Delivery Reviews (1996) 19, 115. Carbamate prodrugs of hydroxy and amino groups are also included, as are carbonate prodrugs and sulfate esters of hydroxy groups. Derivatization of hydroxy groups as (acyloxy)methyl and (acyloxy)ethyl ethers wherein the acyl group may be an alkyl ester, optionally substituted with groups including, but not limited to, ether, amine and carboxylic acid functionalities, or where the acyl group is an amino acid ester as described above, are also encompassed. Prodrugs of this type are described in R. P. Robinson et al., J. Medicinal Chemistry (1996)39, 10.

### DETAILED DESCRIPTION OF THE INVENTION

Compounds of the formula I and their pharmaceutically acceptable salts and solvates may be prepared as described below. Unless otherwise indicated, R¹, R², R³, R⁴, R⁵, R¹⁰, R¹¹, m, p and v are as defined above.

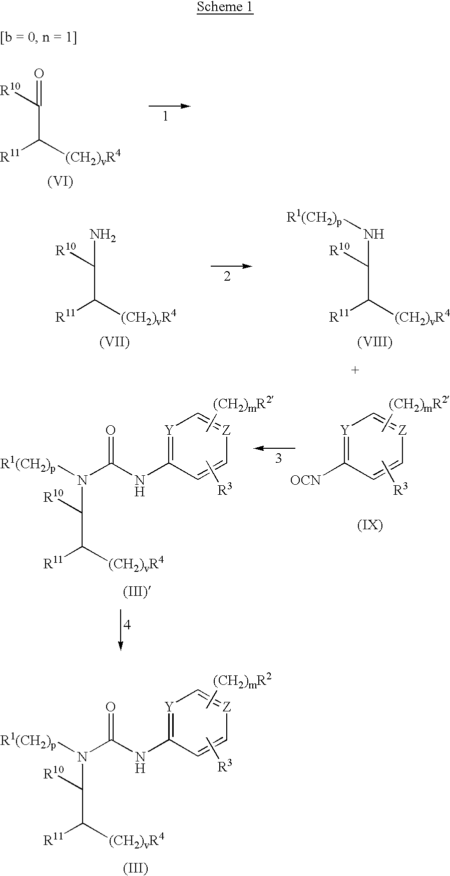

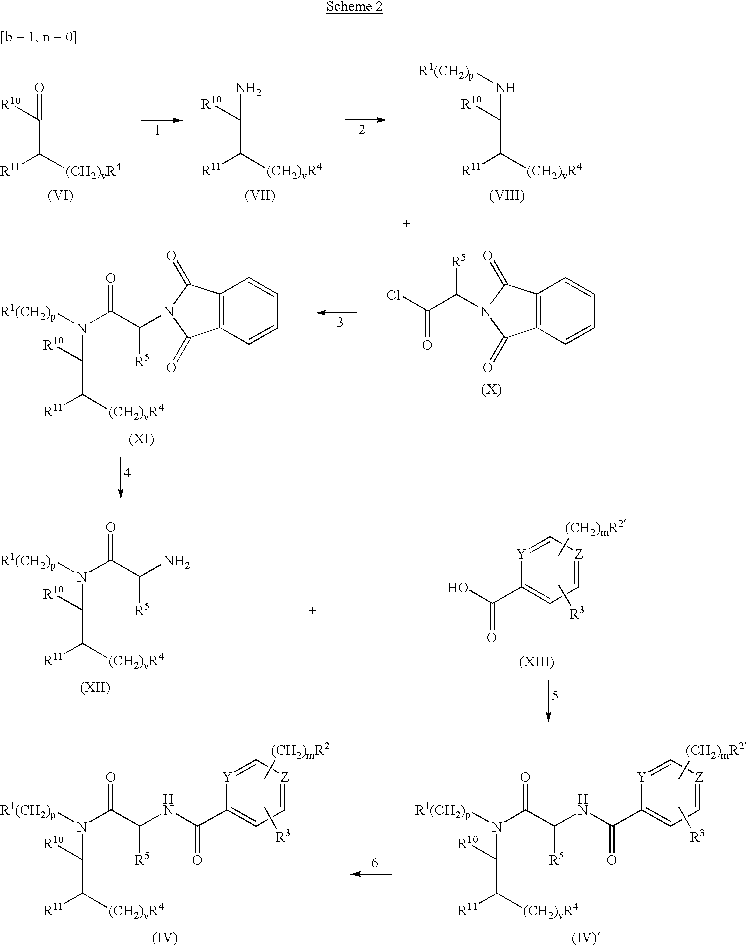

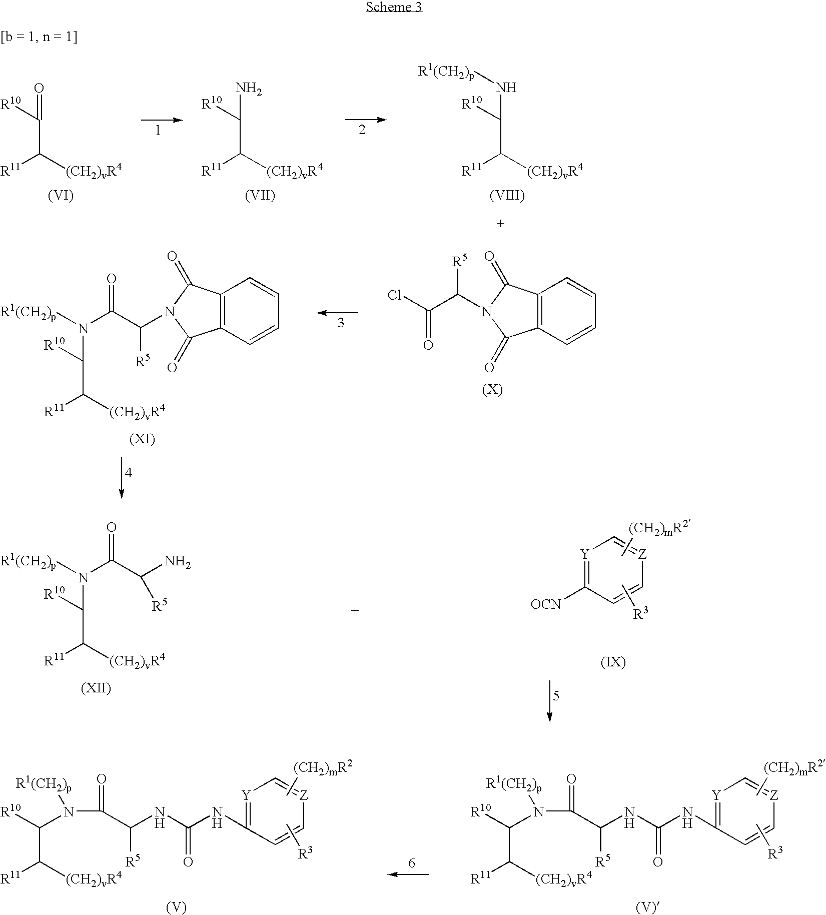

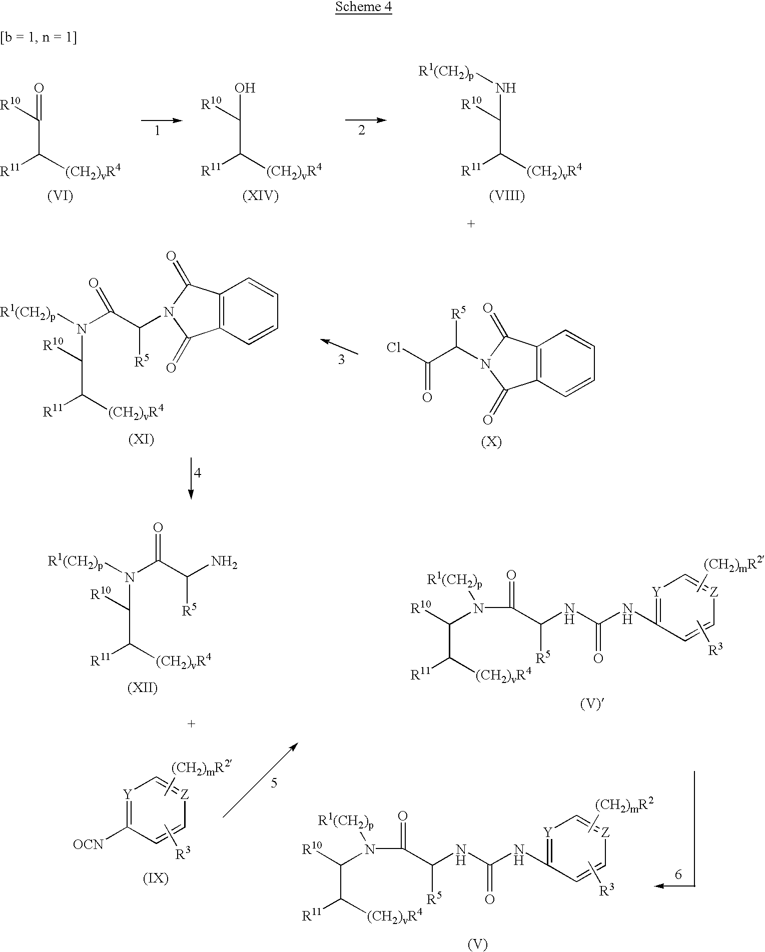

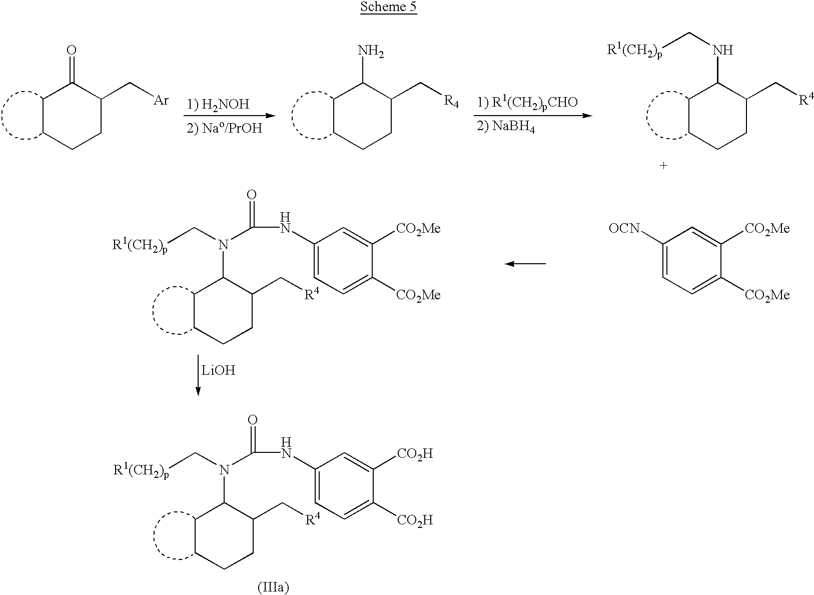

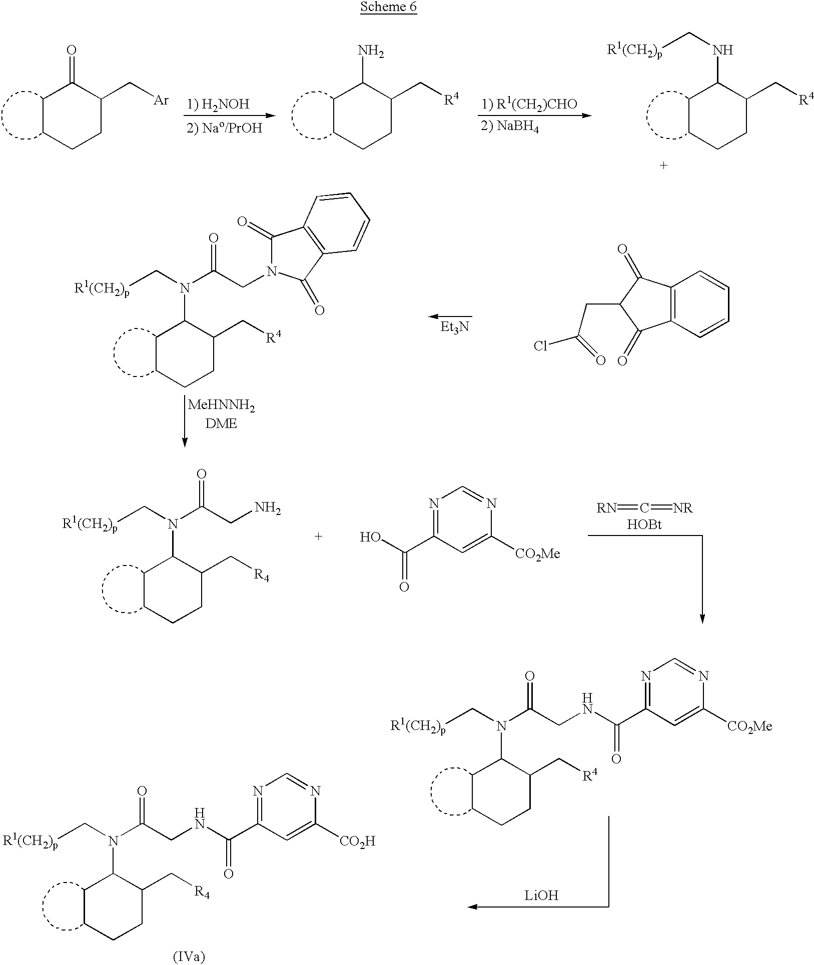

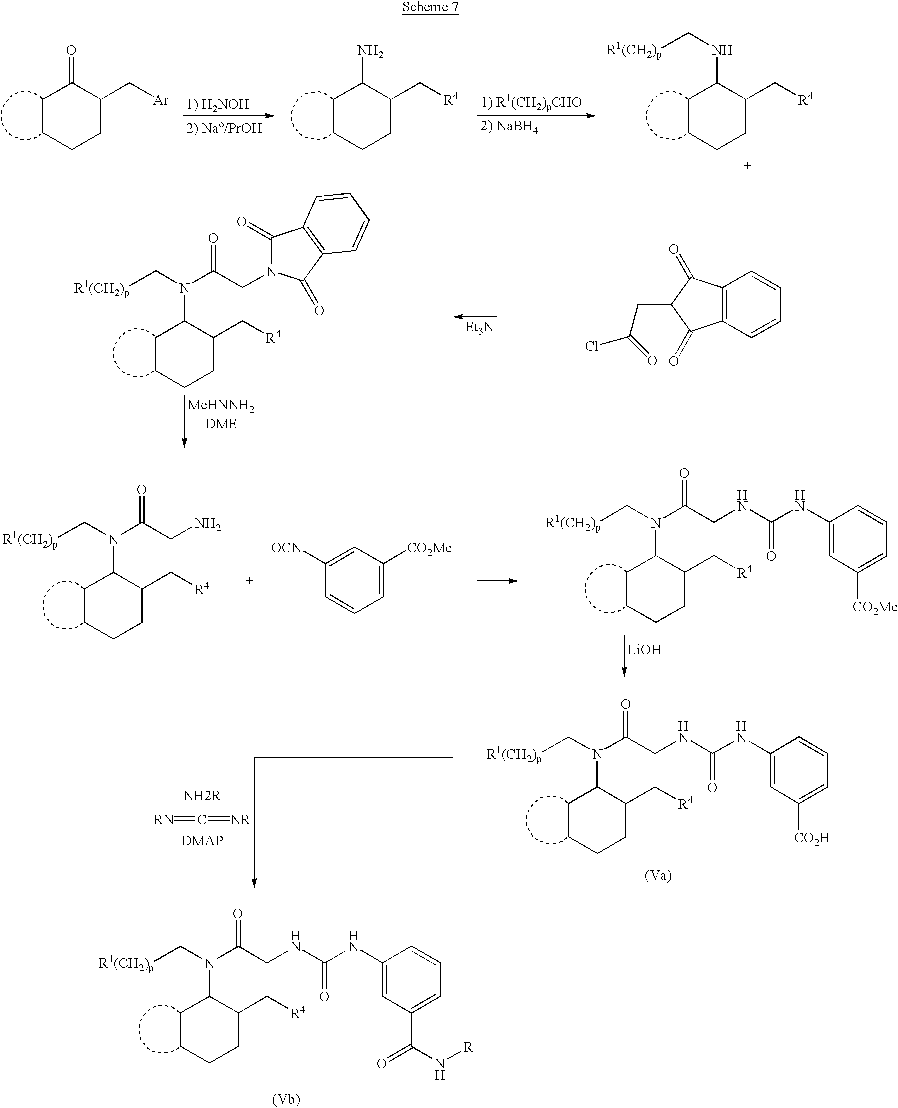

The compounds of the present invention are readily prepared by following the procedures outlined in the schemes illustrated above and typical synthetic procedures familiar to those skilled in the art. Scheme 1 illustrates the preparation of a compound of formula I in which b=0 and n=1. In step 1 of Scheme 1, the compound of formula VII may be prepared in the following manner. First, the compound of formula VI is treated with H₂NOH in a suitably strong base, such as an alkali metal salt of a carboxylic acid, preferably sodium acetate, or an amine base (e.g., ammonia) and a protic solvent, such as an alcohol, preferably ethanol, at a temperature ranging from about 0° C. to about 150° C., preferably between about 20° C. and about 80° C., for a period of from about 1 hours to about 48 hours, to yield an oxime. Thereafter, the oxime so formed may be reduced to the compound of formula VII by dissolving the oxime in an alcoholic solvent, preferably a low molecular weight alcohol such as isopropanol, then heating to boiling and adding a sodium metal, at a temperature ranging from about 0° C. to about 150° C., preferably between about 50° C. and about 100° C., for a period of from about 2 hours to about 48 hours. In step 2 of Scheme 1, the compound of formula VII may be reacted at a temperature ranging from about 0° C. to about 150° C., preferably between about 20° C. and about 110° C., for a period of about 0.5 hours to about 24 hours, with an aldehyde of the formula RCHO, wherein R is (C₁-C₆)alkyl, to form an intermediate imine, which in turn is reduced to a secondary amine (of formula VIII) using a hydride source such as sodium borohydride.

In step 3 of Scheme 1, the compound of formula III may be prepared in the following manner. A compound of formula VII is reacted with an isocyanate of the formula IX, wherein R²′ is a nitrogen protected, oxygen protected, or enol protected analog of the corresponding compound wherein R²′ is replaced by R². The protected R²′ group will be different depending on the identity of R² (in the final desired compound of formula III). For example, when R² is a carboxyclic acid, R²′ will be a carboxylic acid ester. When R² is a group of the formula (c), (d), (f), (g), (h), (j), (k), (l), (o), (p), (f), (v), (w), (x), (aa), (bb), (cc) or (dd), R²′ will contain a nitrogen protecting group. When R² is a group of the formula (a), (b), (e), (i), (m), (n), (r) or (s), R²′ will contain an enol protecting group. When R² is a group of the formula (q) or (v), R² will contain an enol and a nitrogen protecting group. When R²′ is a group of the formula (y) or (z), R²′ will contain an oxygen protecting group. Appropriate nitrogen, enol and oxygen protecting groups will be obvious to those of skill in the art. See Theodora W. Greene and Peter G. M. Wuts, Protective Groups in Organic Synthesis, Second Edition, John Wiley & Sons, Inc., New York 1991.

The reaction of the compound of formula VIII with the isocyanate of formula IX is generally carried out at a temperature ranging from about −20° C. to about 120° C., preferably between about 0° C. and about 80° C., for a period of about 0.5 hours to about 48 hours. This reaction produces the corresponding ester or the corresponding nitrogen, oxygen or enol protected compound of formula III′, which can then be converted into the desired compound of formula III, in which R²′ is replaced by R², by base hydrolysis (preferably using LiOH) in cases where R² is CO₂H, SO₂H or PO₃H₂, or by removal of the respective protecting group(s) in cases where R² is a group of the formula (a) through (dd).

Compounds of the formula III wherein R² is a group of the formula CONHSO₂R¹ or CONR¹(CH₂)CO₂H can be prepared using methods well known to those of skill in the art from the corresponding compounds wherein R² is CO₂H.

Scheme 2 illustrates a method of preparing the compounds of formula I wherein b=1 and n=0. In steps 1 and 2 of Scheme 2, a compound of formula VIII is derived as in Scheme 1. In step 3 of Scheme 2, the secondary amine moiety of formula VIII may be acylated with an activated acid (e.g., an acid chloride) of a nitrogen protected glycine derivative, in the presence of a base at a temperature ranging from about −20° C. to about 150° C., preferably between about 20° C. and about 100° C., for a period of about 1 hour to about 48 hours, to form a compound of formula XI. In step 4 of Scheme 2, the nitrogen protecting group (in the case shown, phthalimide) may be cleaved, using methods known to those of skill in the art, to reveal the basic, nucleophilic nitrogen moiety and provide a compound of formula XII. In step 5 of Scheme 2, the amine moiety of the compound of formula XII may be reacted with a carboxylic acid of formula XIII under conditions suitable to form an amido pyrimidine carboxylic acid ester (for example, in the presence of an amide-forming reagent, such as a mixed carbodiimide/hydroxylbenzoltriazole) reagent, at a temperature ranging from about 0° C. to 100° C., preferably between about 20° C. and about 80° C., for a period of about 1 hour to about 48 hours, followed by base hydrolysis to provide a compound of formula IV′, wherein R² is defined as above. The compound of formula IV′ is then converted, in step 6, into a compound of the formula IV by a method similar to that described above for converting compounds of formula III′ into compounds of the formula III.

Scheme 3 illustrates a process for forming a compound of formula I wherein b=1 and n=1. In steps 1 through 4 of Scheme 3, a compound of formula XII can be derived in the same manner as in steps 1 through 4 of Scheme 2. In steps 5 and 6 of Scheme 3, the. compound of formula V may be formed using a process analogous to that described above for converting compounds of the formula VIII into compounds of the formula III as shown in Scheme 1.

Scheme 4 shows an alternative synthesis of a compound of formula I wherein b=1 and n=1. In step 1 of Scheme 4, the ketone of formula VI is reduced to the alcohol (predominantly cis when R¹⁰ and R¹¹ taken together form a ring, e.g., 7:1 to >15:1 Cis to Trans) with a hydride source (e.g., a borohydride/CeCl₃ mixed reagent). In step 2 of Scheme 4, the secondary alcohol of formula XIV is converted to an azide via a Mitsunobu-type reaction with inversion of stereochemistry; the azide is reduced to the corresponding primary amine with a hydride source (e.g., a borohydride/Ni(OAc)₂ mixed reagent); and the primary amine is reacted with an aldehyde to form an intermediate imine that is in turn reduced to a secondary amine with a hydride source (e.g., a polymer-bound borohydride reagent) to provide a compound of formula VIII. A compound of formula V may then be formed from the compound of formula VIII as in steps 3 through 6 of scheme 3.

Schemes 5, 6 and 7 are more specific schemes for forming certain compounds of formulae III, IV and V, indicated by the formulae designations IIIa, IVa and Va, respectively. The process of Scheme 5 is carried out in a manner similar to the above described process of Scheme 1. The process of Scheme 6 is carried at in a manner similar to the above described, process of Scheme 2. The process of Scheme 7 is carried out in a manner similar to the above described process of Scheme 3. Scheme 7 further illustrates the optional coupling of a functionalized amino derivative with the activated carboxylate of formula Va (generated in this Scheme via a carbodiimide) to provide the corresponding amine of formula Vb.

The curved dashed lines in the compounds of Schemes 5, 6 and 7 refer to an optionally present ring that is fused to the ring formed by R¹⁰ and R¹¹.

The Formula IIIa, wherein the fused ring is naphthalen-1-yl and each of R¹ and R⁴ is phenyl is the compound of Example 21. Formula IVa, wherein the ring is cyclohexyl and each of R¹ and R⁴ is phenyl, is the compound of Example 18. Formula Va, wherein the fused ring is naphthalen-1-yl, and each of R¹ and R⁴ is phenyl, defines the compound of Example 1. Formula Vb, wherein the fused ring is naphthalen-1-yl, and each of R¹ and R⁴ is phenyl, may define the compound of Example 2 or 3 depending on the amine used.

The compounds of the present invention may have asymmetric carbon atoms. Such diasteromeric mixtures can be separated into their individual diastereomers on the basis of their physical chemical differences by methods known to those skilled in the art, for example, by chromatography or fractional crystallization. Enantiomers can be separated by converting the enantiomeric mixtures into a diastereomeric mixture by reaction with an appropriate optically active compound (e.g., alcohol), separating the diastereomers and converting (e.g., hydrolyzing) the individual diastereomers to the corresponding pure enantiomers. All such isomers, including diastereomer mixtures and pure enantiomers are considered as part of the invention.

The compounds of formula I that are basic in nature are capable of forming a wide variety of different salts with various inorganic and organic acids. Although such salts must be pharmaceutically acceptable for administration to animals, it is often desirable in practice to initially, isolate the compound of formula I from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

Those compounds of formula I that are acidic in nature, are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of formula I. Such non-toxic base salts include those derived from such pharmacologicaily acceptable cations as sodium, potassium, calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

Included in the present invention are compounds identical to the compounds of formula I but for the fact that one or more hydrogen or carbon atoms are replaced by isotopes thereof. Such compounds are useful as research and diagnostic tools in metabolism pharmokinetic studies and in binding assays. Specific applications in research include radioligand binding assays, autoradiography studies and in vivo binding studies. Included among the radiolabelled forms of the compounds of formula I are the tritium and C¹⁴ isotopes thereof.

The in vitro activity of the compounds of formula I in inhibiting VEGF/GAG (glycosaminoglycan) binding may be measured using the assay described in U.S. Pat. No. 5,795,860, the subject matter of which is incorporated herein by reference.

Using mixed cellulose ester 96-well filter plates, 150 μL of Dulbeccos PBS (phosphate buffered saline) containing 10% ovalbumin is added. Compound (5.0 μL) is added at a final concentration of 1.8 μM. Compounds are dissolved in 8% DMSO (final DMSO concentration is 0.16%) and tested at concentrations of 32, 10, 3.2, 1.0, 0.32 and 0.10 μM. A mixture of [¹²⁵I]heparin-16-mer (4500 cpm per well), purified VEGF₁₆₅ (20 nM final concentration/well, prepared by Repligen, Inc.) and Dulbeccos PBS with 10% ovalbumin is added to the 96-well plate in a volume of 100 μL. Nonspecific binding is defined using 10 μM heparin-sodium from porcine intestinal mucosa. The assay plate is incubated for 60 minutes at room temperature, filtered using a Millipore filtration apparatus, the plastic bottom plate is removed and the filter plate is allowed to completely dry. The plate bottom is sealed with plastic plate seal and 25 μL of scintillation cocktail is added to each well. The top plate is sealed and is counted for radioactivity on a Microbeta Scintillation Counter. The assay is run in a final volume of 250 μL.

The activity of the compounds of formula I, in vivo, can be determined by the amount of inhibition of tumor growth by a test compound relative to a control. The tumor growth inhibitory effects of various compounds are measured according to the methods of Corbett T. H., et al. “Tumor Induction Relationships in Development of Transplantable Cancers of the Colon in Mice for Chemotherapy Assays, with a Note on Carcinogen Structure”, Cancer Res., 35, 2434-2439 (1975) and Corbett, T. H., et al., “A Mouse Colon-tumor Model for Experimental Therapy”, Cancer Chemother. Rep. (Part 2)”, 5, 169-186 (1975), with slight modifications. Tumors are induced in the flank by s.c. injection of 1×10⁶ log phase cultured tumor cells suspended in 0.1-0.2 ml PBS. After sufficient time has elapsed for the tumors to become palpable (5-6 mm in diameter), the test animals (athymic mice) are treated with active compound (formulated by dissolution in appropriate diluent, for example water or 5% Gelucire™ 44/14 rn PBS by the intraperitoneal (ip) or oral (po) routes of administration once or twice daily for 4 to 10 consecutive days. In order to determine an anti-tumor effect, the tumor is measured in millimeters with Vernier calipers across two diameters and the tumor volume (mm³) is calculated using the formula: Tumor weight=(length×[width]²)/2, according to the methods of Geran, R. I., et al. “Protocols for Screening Chemical Agents and Natural Products Against Animal Tumors and Other Biological Systems”; Third Edition, Cancer Chemother. Rep., 3, 1-104 (1972). The flank site of tumor implantation provides reproducible dose/response effects for a variety of chemotherapeutic agents, and the method of measurement (tumor diameter) is a reliable method for assessing tumor growth rates.

Administration of the compounds of the present invention (hereinafter the “active compound(s)”) can be effected by any method that enables delivery of the compounds to the site of action. These methods include oral routes, intraduodenal routes, parenteral injection (including intravenous, subcutaneous, intramuscular, intravascular or infusion), topical, and rectal administration.

The amount of the active compound administered will be dependent on the subject being treated, the severity of the disorder or condition, the rate of administration and the judgement of the prescribing physician. However, an effective dosage is in the range of about 0.001 to about 100 mg per kg body weight per day, preferably about 1 to about 35 mg/kg/day, in single or divided doses. For a 70 kg human, this would amount to about 0.05 to about 7 g/day, preferably about 0.2 to about 2.5 g/day. In some instances, dosage levels below the lower limit of the aforesaid range may be more than adequate, while in other cases still larger doses may be employed without causing any harmful side effect, provided that such larger doses are first divided into several small doses for administration throughout the day.

The active compound may be applied as a sole therapy or may involve one or more other anti-tumour substances, for example those selected from, for example, mitotic inhibitors, for example vinblastine; alkylating agents, for example cis-platin, carboplatin and cyclophosphamide; anti-metabolites, for example 5-fluorouracil, cytosine arabinoside and hydroxyurea, or, for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; enzymes, for example interferon; and anti-hormones, for example anti-estrogens such as Nolvadex™ (tamoxifen) or, for example anti-androgens such as Casodex™ (4′-cyano-3-(4-fluorophenylsulphonyl)-2-hydroxy-2-methyl-3′-(trifluoromethyl)propionanilide). Such conjoint treatment may be achieved by way of the simultaneous, sequential or separate dosing of the individual components of the treatment.

The pharmaceutical composition may, for example, be in a form suitable for oral administration as a tablet, capsule, pill, powder, sustained release formulations, solution, suspension, for parenteral injection as a sterile solution, suspension or emulsion, for topical administration as an ointment or cream or for rectal administration as a suppository. The pharmaceutical composition may be in unit dosage forms suitable for single administration of precise dosages. The pharmaceutical composition will include a conventional pharmaceutical. carrier or excipient and a compound according to the invention as an active ingredient. In addition, it may include other medicinal or pharmaceutical agents, carriers, adjuvants, etc.

Exemplary parenteral administration forms include solutions or suspensions of active compounds in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical compositions may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials, therefore, include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

Methods of preparing various pharmaceutical compositions with a specific amount of active compound are known, or will be apparent, to those skilled in this art. For examples, see Remington's Pharmaceutical Sciences, Mack Publishing Company, Easter, Pa., 15th Edition (1975).

The examples and preparations provided below further illustrate and exemplify the compounds of the present invention and methods of preparing such compounds. It is to be understood that the scope of. the present invention is not limited in any way by the scope of the following examples and preparations.

### Preparation A

### 2-Benzyl-3,4-dihydro-2H-naphthalen-1-one oxime

To a solution of 2-benzyl-1-tetralone (prepared according to Org. Prep. Proc. Inter., 2, 37 (1970)), (58.95 g, 0.250 mol) and 95% ethanol (500 mL) was added sodium acetate (45.1 g, 0.55 mol) and hydroxylamine hydrochloride (20.8 g, 0.30 mol). The reaction was heated to reflux overnight and then allowed to cool to room temperature and concentrated under vacuum. The residue was partitioned between water (300 mL) and ethyl acetate (500 mL), the organic layer was then sequentially washed with water (300 mL), and brine (500 mL). This was then dried over sodium sulfate and concentrated to give 61.1 g of an orange solid. Recrystalization from ethyl acetateihexanes gave the title compound as a white solid, m.p. 123-124° C.

¹HNMR (CDCl₃, δ): 1.8 (m, 2H), 2.61 (dd, J=11.4, 13.5 Hz, 1H), 2.69 (m, 1H), 3.03 (ddd, J=6.0, 11, 17 Hz, 1H), 3.16 (dd, J=4.1, 13 Hz, 1H), 3.87 (ddd, J=4.0, 4.0, 11 Hz, 1H), 7.1-7.3 (m, 8H), 7.95 (d, J=7.7Hz, 1H), 8.82 (br s, 1H).

¹³C-NMR (CDCl₃, δ): 23.6, 25.0, 33.8, 34.5, 124.5, 126.2, 126.5, 128.4, 129.0, 129.2, 129.3, 129.9, 138.7, 140.1, 158.1.

MS, APCl (%): 252 (M+1, 100), 234 (25)

### Preparation B

### 1-Amino-2-benzyltetralin

To a solution of the oxime from preparation A (15.0 g, 59.7 mmol) and dry isopropanol (1 L) which had been heated to reflux was added sodium metal spheres (74 g, 3.2 mol) over 1 h. The reaction mixture was heated to reflux for an additional 4 h, and then allowed to stand at room temperature overnight. Water (500 mL) was cautiously added to the now solid reaction mixture, and concentrated HCl was added until pH 1. The mixture was concentrated to a solid and then partitioned between water (500 mL) and ether (1 L). The remaining aqueous solution was made basic with solid NaOH and then extracted with ether (3×500 mL). These latter combined organic layers were washed with brine (500 mL), dried (K₂CO₃) and concentrated to give 10.5 g of the title compound (1:1 mixture of cis/trans isomers) as a pale yellow oil.

¹H-NMR (CDCl₃, δ): 1.4-2.2 (m, 3H), 2.49 (dd, J=8.9, 14 Hz, 1H), 2.6-2.9 (m), 2.96 (dd, J=5.5, 14 Hz, 1H), 3.70 (d, J=5.6 Hz, 1H), 3.81 (d, J=3.5 Hz, 1H).

MS, APCl (%): 237 (M+1, 6), 221 (100).

### Preparation C

### 1-Benzylamino-2-benzyltetralin

To a solution of the product of preparation B (10.5 g, 44.4 mmol) and toluene (200 mL) was added benzaldehyde (4.74 mL, 46.6 mmol) and p-toluenesulfonic acid (1.0 g, 5.3 mmol). The reaction was heated to reflux for 18 h and the water produced was collected in a Dean-Stark trap. After cooling, the reaction was partitioned between NaHCO₃ (2% aqueous, 200 mL) and ethyl acetate (500 mL), dried (Na₂SO₄) and concentrated to afford an intermediate imine which was dilute immediately with dry methanol (400 mL). Reduction was effected by the portionwise addition of NaBH4 (10.0 g, 0.264 mol) and stirring was continued for 3 h at room temperature. The reaction mixture was concentrated to dryness and partitioned between water (300 mL) and ethyl acetate (500 mL). The organic layer was dried (Na₂SO₄) and concentrated to afford a cis/trans isomeric mixture of the title compound. Chromatography of this product on silica gel with ethyl acetate/hexanes mixture afforded the separate isomers:

Less polar diastereomer, assigned cis stereochemistry based on an X-ray crystal, 5.00 g (34%), as an oil.

¹H-NMR (CDCl₃, δ): 1.7-1.9 (m, 2H), 2.2 (m, 1H), 2.75 (m, 2H), 2.94 (ddd, J=3.5, 6.5, 17 Hz, 1H), 3.03 (dd, J=7.0, 13 Hz, 1H), 3.61 (d, J=3.5 Hz, 1H), 3.83 (d, J=12.9 Hz, 1H), 3.96 (d, J=12.9 Hz, 1H), 7.1-7.5 (m, 14H).

MS, APCl (%): 328 (M+1, 40), 221 (100).

More polar trans diastereomer, 5.27 g (36%), as an oil.

¹H-NMR (CDCl₃, δ): 1.6 (m, 2H), 2.10 (m, 1H), 2.3 (m, 1H), 2.49 (dd, J=8.3 13 Hz, 1H), 2.8 (m, 2H), 3.58 (d, J=4.3 Hz, 1H), 3.68 (d, J=13.2 Hz, 1H), 3.74 (d, J=13.2 Hz,1H), 7.0-7.4 (m, 14H).

MS, APCl (%): 328 (M+1, 30), 221 (100).

### Preparation D

### trans-N-Benzyl-N-(2-benzyl-1,2,3,4-tetrahydro-naphthalen-1-yl)-2-(1,3-dioxo-1,3-dihydro-isoindol-2-yl)-acetamide

To a 25 mL round-bottomed flask equipped with N₂ inlet were added the product of preparation C (trans isomer, 510 mg, 1.56 mmol), methylene chloride (5 mL), triethylamine (217 uL, 1.56 mmol), 4-dimethylaminopyridine (10 mg), and 348 mg (1.56 mmol) phthalimidoglycine acid chloride (prepared according to Org. Syn., 72, 15 (1994)). The reaction was stirred at r.t. 14 h, another100 mg phthalimidoglycine acid chloride and 20 mg 4-dimethylaminopyridine added, and stirring continued for 3 h. The reaction was then poured into dilute aqueous sodium bicarbonate solution, extracted with ethyl acetate, and the organic layer washed with. brine, dried (Na₂SO₄), and concentrated. The residue. was chromatographed on silica gel using hexane/ethyl acetate as eluant to afford 340 mg (42%) of the product as a low-melting solid.

¹H-NMR (CDCl₃, δ): 1.3-2.1 (m, 2H), 2.17, 2.36 (t, 1H), 2.5-2.8 (m, 2H), 3.20 (t, 1H), 3.55, 4.11 (d, 1H), 4.5-4.9 (m, 3H), 5.20, 5.94 (d, 1H), 6.35 (br s, 1H), 6.80 (d, 1H), 7.0-7.5 (m, 14H), 7.7 (m, 2H), 7.9 (m, 2H).

MS, APCl (%): 515 (M+1, 50), 295 (100).

### Preparation E

### trans-2-Amino-N-benzyl-N-(2-benzyl-1,2,3,4-tetrahydro-naphthalen-1-yl)-acetamide

To a 25 mL round-bottomed flask equipped with N₂ inlet were added the product of preparation D (340 mg, 0.661 mmol), 1,2-dimethoxymethane (5 mL), and N-methylhydrazine (176 uL, 3.31 mmol). The solution was heated at 70 ° C. for 14 h, cooled, and evaporated. The residue was chromatographed on silica gel using methanol/methylene chloride as eluant to afford 360 mg of a material contaminated with byproduct phthalazinedione, and used directly in the following step.

¹H-NMR (DMSO-_{D6}, δ): 1.2-1.5 (m, 2H), 1.8 (m, 1H) 2.0-3.6 (multiplets, 6H), 4.20 (AB_{q}, J=18, Δν=48, 2H), 4.4-4.9 (multiplets, 4H), 6.9-7.2 (m, 14H).

MS; APCl (%): 385 (M+1, 100), 206 (70), 165 (30).

### Preparation F

### trans-3-(3-{[Benzyl-(2-benzyl-1,2,3,4-tetrahydro-naphthalen-1-yl)-carbamoyl]-methyl}-ureido)-benzoic Acid Methyl Ester

To a solution of monomethyl isophthalate (0.23 g, 0.60 mmol), benzene (10 mL) and diisopropyl ethylamine (0.136 mL, 0.781 mmol was added diphenylphosphoryl azide (0.168 mL, 0.780 mmol) and the solution was heated at reflux for 2 h. The product of preparation E (0.23 gm, 0.60 mmol) was added as a solution with benzene (1 mL) and the heating was continued overnight. After cooling to room temperature, the reaction was concentrated and chromatographed on silica gel utilizing ethyl acetate/methylene chloride as eluent to give the title compound (218 mg, 64%) as a white foam.

¹H-NMR (DMSO-_{D6}, δ): 1.2-1.5 (m, 2H), 1.8 (m, 1H), 2.6 (m, 1H), 2.9 (m, 1H)), 3.31 (s, 3H), 3.47 (d, J=15 Hz, 1H), 3.8 (m, 2H), 4.0-5.0 (multiplets, 4H), 6.28 (br s, 1H), 6.55, 6.7 (m, 1H), 7.0-7.7 (m, 17H), 8.12, 8.20 (m, 1H), 8.97, 9.17, 9.24 (s, 1H).

MS, APCl (%): 562 (M+1, 100), 385 (50), 342 (90), 169 (60).

### EXAMPLE 1

### trans-3-(3-{[Benzyl-(2-benzyl-1,2,3,4-tetrahydro-naphthalen-1-yl)-carbamoyl]-methyl}-ureido)-benzoic acid

To a solution of the product of preparation F (145 mg, 0.258 mmol), methanol (30 mL) and water (5 mL) was added lithium hydroxide (100 mg, 2.38 mmol). After heating to reflux for 16 h, the solution was cooled and concentrated under vacuum. 1N HCl (10 mL) was added and the aqueous layer was extracted with ethyl acetate (5×20 mL), the combined organic layers were then washed with brine, dried (Na₂SO₄), and concentrated to give a solid (45 mg). The title compound was isolated (25 mg) as a white solid following trituration with ethyl acetate/hexanes (mp>250° C.).

¹H-NMR (DMSO-_{D6}, δ): 1.2-1.5 (m, 2H), 1.8 (m, 1H), 2.6 (m, 2H), 2.9 (m, 1H), 3.49 (d, J=15 Hz, 1 H), 3.8 (m, 2H), 4.0-5.0 (multiplets, 4H), 6.29 (br s, 1 H), 6.53, 6.7 (m, 1H), 7.0-8.2 (m, 18H), 9.09, 9.18 (s, 1H).

MS, APCl (%): 548 (M+1, 80), 385 (50), 328 (60), 169 (100).

### EXAMPLE 2

### trans-2-[3-(3-Benzenesulfonylaminocarbonyl-phenyl)-ureido]-N-benzyl-N-(2-benzyl-1,2,3,4-tetrahydro-naphtha en-1-yl)-acetamide

To a solution of the product of Example 1 (0.24 g, 0.44 mmol), benzene sufonamide (83 mg, 0.53 mmol), 4-dimethylaminopyridine (64 mg, 0.53 mmol) and methylene chloride (10 mL) was added 1-(3-dimethylaminopropyl)-3-ethyl-carbodiimide hydrochloride (101 mg, 0.53 mmol). After stirring at room temperature overnight, the reaction was diluted with ethyl acetate (200 mL) and washed with 1N HCl (50 mL), brine (50 mL) , dried (Na₂SO₄), and concentrated to give a solid. Chromatography on silica gel (methanol/methylene chloride) and recrystalization from benzene/cyclohexane gave the title compound (50 mg) as a white solid (mp 120-121° C.).

¹H-NMR (DMSO-_{D6}, δ): 1.2-1.5 (m, 2H), 1.8 (m, 1H), 2.6 (m, 2H), 2.9 (m, 1H), 3.46 (d, J=15 Hz, 1H), 4.0-5.0 (multiplets, 6H), 6.27 (br s, 1H), 6.54, 6.7 (m, 1H), 7.0-8.0 (m, 23H), 9.06, 9.13 (s, 1H).

MS, APCl (%): 685 (M−1, 100), 670 (35), 545 (40), 156 (70).

### EXAMPLE 3

### trans-3-(3-{[Benzyl-(2-benzyl-1,2,3,4-tetrahydro-naphthalen-1-yl)-carbamoyl]-methyl}-ureido)-N-(1H-tetrazol-5-yl)-benzamide

In a procedure similar to Example 2, utilizing aminotetrazole (rather than benzensulfonamide), the title compound was isolated as a white solid, following recrystalization from benzene/cyclohexane (mp 155° C., decomposition).

¹H-NMR (DMSO-_{D6}, δ): 1.2-1.6 (m, 2H), 1.8 (m, 1H), 2.2 (m, 1H), 2.6 (m, 2H), 2.9 (m, 1H), 3.5 (m, 2H), 4.0-5.0 (multiplets, 4H), 6.27 (br s, 1H), 6.58, 6.70 (m, 1H), 7.0-8.2 (m, 18H), 9.14, 9.22 (s, 1H).

MS, APCl (%): 613 (M−1, 30), 175 (100).

### Preparation G

### 3-Tetrazolylbenzoic acid

To a 500 mL round-bottomed flask equipped with condenser and N2 inlet were added 10 g (68 mmol) 3-cyano benzoic acid and 225 mL methanol. To the stirring solution was added 7 mL acetyl chloride carefully, and the reaction heated at 55° C. for 12 hr, then stirred at room temperature overnight. The reaction was concentrated, partitioned between water and ethyl acetate, washed with aqueous sodium bicarbonate, water, and brine, and dried over sodium sulfate. After evaporation, the residue was crystallized from isopropanol to give 8.32 g (76%).

¹H-NMR (δ, CDCl₃): 3.93 (s, 3H), 7.56 (t, J=7, 1H), 7.82 (m, 1H), 8.23 (m, 1H), 8.30 (m, 1 H).

IR (neat, cm.⁻¹): 2228 (CN) cm.⁻¹.

The nitrile, 3.4 g (21 mmol), was dissolved in 50 mL xylene, treated with 11.0 g (52.7 mmol) of trimethylstannyl azide, and refluxed 3 hr. The reaction was cooled, poured into 5 N hydrochloric acid, shaken vigorously after addition of ethyl acetate, and the organic layer separated and washed with water and brine. After drying over sodium sulfate and evaporation, the residue (particle beam MS showed P=205 for parent+1 peak, IR showed no peak at 2228 cm.⁻¹) was taken up in 30 mL tetrahydrofuran and treated with a solution of 1.04 g (24.84 mmol) lithium hydroxide in 15 mL water and then enough methanol (8 mL) to give a solution. After stirring at room temperature for 20 hr, the reaction was concentrated, partitioned between ethyl acetate and 1 N hydrochloric acid. The organic layer was washed with water and brine, dried over sodium sulfate, and evaporated to a white solid, 1.7 g (91%).

¹H-NMR (δ, CDCl₃): 7.70 (t, J=8, 1H), 8.22 (m, 2H), 8.69 (bs, 1H).

IR (neat, cm.⁻¹): 1671 (C═O) cm.⁻¹.

MS (particle beam, %): 208 (parent+NH₄⁺, 100), 191 (parent+1, 20).

### EXAMPLE 4

### trans-N-{[Benzyl-(2-benzyl-1,2,3,4-tetrahydro-naphthalen-1-yl)-carbamoyl]-methyl}-3-(1H-tetrazol-5-yl)-benzamide

In a procedure similar to preparation F, utilizing the product of preparation G (rather then monomethyl isophthalate), the title compound was isolated following chromatography (ethyl acetate/methylene chloride/acetic acid) and recrystallization from benzene/cyclohexane (mp 119° C., decomposition).

¹H-NMR (DMSO-_{D6}, δ): 1.2-1.6 (m, 2H), 1.8 (m, 1H), 2.2 (m, 1H), 2.6 (m, 2H), 2.9 (m, 1 H), 3.47 (d, J=15 Hz, 1 H), 4.0-5.2 (multiplets, 6H), 6.28 (br s, 1 H), 6.66 (m, 1H), 7.0-8.22 (m, 18H), 8.63 (m, 1H), 9.10 (m, 1H).

MS, APCl (%): 555 (M−1, 100).

### Preparation H

### trans-[3-(3-{[Benzyl-(2-benzyl-1,2,3,4-tetrahydro-naphthalen-1-yl)-carbamoyl]-methyl}-ureido)-phenyl]-acetic acid methyl ester

To a 0° C. solution of 3-amino-phenyl acetic acid methyl ester (112 mg, 0.68 mmol), triethylamine (0.070 mL) and THF (10 mL) was added triphosgene (54 mg, 0.18 mmol) and triethylamine (0.14 mL, 1.56 mmol total). The reaction was stirred for 30 min at room temperature and then a solution of the product of preparation E (200 mg, 0.52 mmol) and THF (10 mL) was added. After stirring overnight the reaction was diluted with ethyl acetate and washed with aqueous acid, water, brine, dried (Na₂SO₄), and concentrated to give a solid. Chromatography on silica gel utilizing ethyl acetate/hexanes gave the title compound as a foam (100 mg).

¹H-NMR (CDCl₃, δ): 1.2-1.6 (m, 2H), 1.8 (m, 1H), 2.1 (m, 1H), 2.6 (m, 2H), 3.01, 3.14 (d, J=10.4 Hz, 1H), 3.55 (d, J=15 Hz, 1H), 3.63 (s, 3H), 4.0-4.6 (multiplets, 3H), 4.85, 5.20 (d, 1H) 6.2-7.4 (m, 18H), 7.67 (s, 1H).

### EXAMPLE 5

### trans-[3-(3-{[Benzyl-(2-benzyl-1,2,3,4-tetrahydro-naphthalen-1-yl)-carbamoyl]-methyl}-ureido)-phenyl]-acetic acid

In a procedure similar to Example 1, the product of preparation H afforded the title compound isolated as an amorphous solid.

¹H-NMR (CDCl₃, δ): 1.2-1.6 (m, 2H), 1.8 (m, 1H), 2.1 (m, 1H), 2.6 (m, 2H), 2.95, 3.11 (d, J=10 Hz, 1H), 3.5 (m, 2H), 3.63 (s, 3H), 4.0-4.6 (multiplets, 3H), 4.85, 5.20 (d, 1H) 6.2-7.9 (m, 20H).

MS (FAB, %): 562 (M+, 10), 342 (15), 165 (30).

| | C | H | N |
|---|---|---|---|
| Calc'd for C₃₅H₃₅N₃O₄·0.5 H₂O | 73.66 | 6.36 | 7.36 |
| found | 74.03 | 6.45 | 7.07 |

### Preparation I

### cis-N-Benzyl-N-(2-benzyl-1,2,3,4-tetrahydro-naphthalen-1-yl)-2-(1,3-dioxo-1,3-dihydro-isoindol-2-yl)-acetamide

To a solution of the cis isomer of preparation C (1.0 g, 3.1 mmol), triethylamine (0.9 mL, 6.1 mmol), 4-dimethylaminopyridine (a few crystals) and THF (50 mL) was added 1.4 g (6.1 mmol) phthalimidoglycine acid chloride (prepared according to Org. Syn., 72, 15 (1994)). The reaction was heated to reflux for 14 h, and then cooled to room temperature and diluted with ethyl acetate (200 mL). The organic solution was washed with water (2×50 mL), brine, dried (Na₂SO₄), and concentrated to give a solid. Chromatography on silica gel utilizing ethyl acetate/hexanes gave the title compound (970 mg, 58%) as a foam.

¹H-NMR (CDCl₃, δ): 1.4 (m, 1H), 1.6 (m, 2H), 2.3 (m, 1H), 2.40 (dd, J=13, 14 Hz, 1H), 2.4-3.0 (m, 2H), 3.20 (dd, J=3.2, 13 Hz, 1H), 4.31 (d, J=16 Hz, 1H), 4.37 (d, J=18 Hz, 1H), 4.45 (d, J=16 Hz, 1H), 4.54 (d, J=18 Hz, 1H), 6.27 (d, J=4.8 Hz, 1H), 7.1-7.9 (m, 18H).

MS (FAB, %): 515 (M+, 10), 295 (25).

### Preparation J

### cis-2-Amino-N-benzyl-N-(2-benzyl-1,2,3,4-tetrahydro-naphthalen-1-yl)-acetamide

In a procedure similar to preparation E, 943 mg of the product of preparation I gave 681 mg of the title compound, isolated as a foam.

¹H-NMR (CDCl₃, δ): 1.4 (m, 1H), 1.6 (m, 2H), 2.3 (m, 1H), 2.40 (dd, J=13, 13 Hz, 1H), 2.4-3.0 (m, 2H), 3.25 (dd, J 3.0, 13 Hz, 1H), 3.97 (d, J=13 Hz, 1H), 4.09 (d, J=17 Hz, 1H), 4.14 (d, J=17 Hz, 1H), 4.68 (d, J=13 Hz, 1H), 5.02 (d, J=5.7 Hz, 1H) 6.34 (d, J=4.8 Hz, 1H), 7.0-7.4 (m, 14H).

MS (FAB, %): 515 (M+, 10), 295 (25).

### Preparation K

### cis-[3-(3-{[Benzyl-(2-benzyl-1,2,3,4-tetrahydro-naphthalen-1-yl)-carbamoyl]-methyl}-ureido)-phenyl]-acetic acid methyl ester

In a procedure similar to preparation H, 200 mg of the product of preparation J afforded 91 mg of the title compound isolated as a foam.

¹H-NMR (CDCl₃, δ): 1.2-1.6 (m, 3H), 2.2-3.2 (multiplets, 4H), 3.5 (m, 1H), 3.61 (s, 3H), 3.8-4.7 (multiplets, 4H), 6.29 (br s, 1 H), 6.6-7.3 (multiplets, 19H), 7.89, 8.02 (s, 1H).

MS (FAB, %): 576 (M+1, 10), 356 (30), 165 (70).

HRMS calc'd for C₃₆H₃₈N₃O₄: 576.2862; found: 576.2888

### EXAMPLE 6

### cis-[3-(3-{[Benzyl-(2-benzyl-1,2,3,4-tetrahydro-naphthalen-1-yl)-carbamoyl]-methyl}-ureido)-phenyl]-acetic acid

In a procedure similar to Example 1, 80 mg of the product of preparation K afforded 56 mg of the title compound isolated as an amorphous solid.

¹H-NMR (CDCl₃, δ): 1.2-1.6 (m, 3H), 2.2-3.2 (multiplets, 4H), 3.5 (m, 1H), 3.8-4.7 (multiplets, 4H), 6.23 (br s, 1H), 6.6-7.3 (multiplets, 18H), 7.5 (m, 1H), 7.89, 8.02 (s, 1H).

MS (FAB, %): 562 (M+1, 5), 342 (10), 165 (30).

| | C | H | N |
|---|---|---|---|
| Calc'd for C₃₅H₃₅N₃O₄·0.5 H₂O | 73.66 | 6.36 | 7.36 |
| found | 73.94 | 6.36 | 7.32 |

### Preparation L

### (2-Benzyl-1,2,3,4-tetrahydro-naphthalen-1-yl)-furan-2-ylmethyl-amine

To a solution of the product of preparation B (1.0 g, 4.2 mmol), methanol (30 mL) and 2-furaldehyde (405 mg, 8.4 mmol) was added sodium cyanoborohydride (529 mg, 8.4 mmol). After stirring at room temperature for 7 days, the solvent was removed under vacuum and the residue diluted with ethyl acetate (100 mL). The organic solution was washed with water (50 mL), brine, dried (Na₂SO₄), and concentrated to give a oil. Chromatography on silica gel utilizing ethyl acetate/hexanes gave the separated isomers:

Less polar diastereomer, assigned cis stereochemistry (685 mg):

More polar diastereomer, assigned trans stereochemistry (352 mg):

¹H-NMR (CDCl₃, δ): 1.6 (m, 1H), 2.1 (m, 1H), 2.3 (m, 1H), 2.48 (dd, J=8.0, 13 Hz, 1H), 2.65 (dd, J=7.2, 13 Hz, 1H), 2.8 (m, 2H), 3.53 (d, J=3.7 Hz), 3.68 (d, J=15 Hz, 1H), 3.75 (d, J=15 Hz, 1H), 5.97 (dd, J=2.7,.3.1 Hz, 1H), 6.27 (dd, J=1.9, 3.1 Hz, 1H), 7.1-7.4 (m, 10H).

MS (FAB, %): 318 (M+1, 30), 221 (40), 129 (40).

### Preparation M

### (2-Benzyl-1,2,3,4-tetrahydro-naphthalen-1-yl)-pyridin-4-ylmethyl-amine

In a procedure similar to preparation L, the title compounds were prepared and separated:

Less polar diastereomer, assigned cis stereochemistry (40%):

¹H-NMR (CDCl₃, δ): 1.8 (m, 2H), 2.2 (m, 1H), 2.73 (dd, J=8.0, 14 Hz, 1H), 2.78 (dd, J=8.7, 14 Hz, 1H), 2.9 (m, 2H), 3.51 (d, J=3.2 Hz), 3.73 (d, J=15 Hz, 1H), 3.91 (d, J=15 Hz, 1H), 7.0-7.3 (m, 13H).

More polar diastereomer, assigned trans stereochemistry (21%):

¹H-NMR (CDCl₃, δ): 1.6 (m, 1H), 2.1 (m, 1H), 2.3 (m, 1H), 2.51 (dd, J=7.5, 13 Hz, 1H), 2.61 (dd, J=8.5, 13 Hz, 1H), 2.7 (m, 2H), 3.56 (d, J=3.6 Hz), 3.65 (d, J=15 Hz, 1H), 3.72 (d, J=15 Hz, 1H), 7.0-7.3 (m, 13H).

MS (FAB, %): 329 (M+1, 40),221 (150), 129 (25), 107(35).

### Preparation N

### (2-Benzyl-1,2,3,4-tetrahydro-naphthalen-1-yl)-isoxazol-5-ylmethyl-amine

To a mixture of the product of preparation B (1.8 g, 7.6 mmol), sodium carbonate (804 mg, 7.6 mmol) and acetonitrile (50 mL) was added isoxazol-5-ylmethyl iodide (1.6 g, 7.6 mmol) and the reaction mixture was then heated at reflux for 6 h. After cooling to room temperature, the solvent was removed under vacuum, and the residue partitioned between water and ethyl acetate. The organic layer solution was washed with water (50 mL), brine, dried (Na₂SO₄), and concentrated to give an oil. Chromatography on silica gel utilizing ethyl acetate/hexanes gave the separated isomers:

Less polar diastereomer, assigned cis stereochemistry (58%):

¹H-NMR (CDCl₃, δ): 1.8 (m, 2H), 2.2 (m, 1H), 2.75 (dd, J=8.3, 14 Hz, 1H), 2.82 (dd, J=8.9, 14 Hz, 1H), 2.9 (m, 2H), 3.50 (d, J=3.4 Hz), 3.85 (d, J=15 Hz, 1H), 4.03 (d, J=15 Hz, 1H), 6.15 (d, J=2.2 Hz, 1H), 7.0-7.4 (m, 9H), 8.18 (d, J=2.2 Hz, 1H).

More polar diastereomer, assigned trans stereochemistry (9%):

¹H-NMR (CDCl₃, δ): 1.6 (m, 1H), 2.1 (m, 1H), 2.3 (m, 1H), 2.51 (dd, J=8.0, 13 Hz, 1H), 2.62 (dd, J=7.2, 13 Hz, 1H), 2.7 (m, 2H), 3.50 (d, J=3.4 Hz), 3.82 (s, 2H), 6.15 (d, J=2.0 Hz, 1H), 7.0-7.3 (m, 13H), 8.12 (d, J=2.0 Hz, 1H).

### Preparation O

### trans-(2-Benzyl-1,2,3,4-tetrahydro-naphthalen-1-yl)-(4-fluoro-benzyl)-amine

In a procedure similar to preparation C, the product of preparation B (1.5 g, 6.3 mmol) was reacted with p-fluorobenzaldehyde (0.746 mL, 6.95 mmol) to give 0.87g of the title compound as an oil:

¹H-NMR (CDCl₃, δ): 1.6 (m, 1H), 2.1 (m, 1H), 2.3 (m, 1H), 2.49 (dd, J=7.8, 13 Hz, 1H), 2.65 (dd, J=7.3, 13 Hz, 1H), 2.7 (m, 2H), 3.63 (t, J=4.4 Hz), 3.61 (d, J=13 Hz, 1H), 3.67 (d, J=13 Hz, 1H), 6.92 (t, J=8.7 Hz, 2H), 7.0-7.3 (m, 11H).

MS, APCl (%): 346 (M+1, 100), 221 (60).

### Preparation P

### trans-(2-Benzyl-1,2,3,4-tetrahydro-naphthalen-1-yl)-(4-methoxy-benzyl)-amine

In a procedure similar to preparation C, the product of preparation B (1.5 g, 6.3 mmol) was reacted with 2-methoxybenzaldehyde (0.840 mL, 6.95 mmol) to give 0.89 g of the title compound as an oil:

¹H-NMR (CDCl₃, δ): 1.6 (m, 1H), 2.1 (m, 1H), 2.3 (m, 1H), 2.44 (dd, J=8.4, 13 Hz, 1H), 2.67 (dd, J=6.7, 13 Hz, 1H), 2.7 (m, 2H), 3.51 (d, J=3.7 Hz), 3.64 (d, J=13 Hz, 1H), 3.77 (d, J=13 Hz, 1H), 3.79 (s, 3H), 6.8-7.3 (m, 13H).

MS, APCl (%): 358 (M+1, 100).

### Preparation Q

### trans-(2-Benzyl-1,2,3,4-tetrahydro-naphthalen-1-yl)-pyridin-2-ylmethyl-amine

In a procedure similar to preparation C, the product of preparation B (1.5 g, 6.3 mmol) was reacted with 2-pyridinecarboxaldehyde (0.661 mL, 6.95 mmol) to give 0.72 g of the title compound as an oil:

¹H-NMR (CDCl₃, δ): 1.6 (m, 1H), 2.1 (m, 1H), 2.3 (m, 1H), 2.46 (dd, J=8.3, 13 Hz, 1H), 2.69 (dd, J=6.8, 13 Hz, 1H), 2.7 (m, 2H), 3.57 (d, J=3.9 Hz), 3.83 (s, 2H),7.0-7.3 (m, 11H), 7.54 (dt, J=1.9, 7.7 Hz, 1H), 8.50 (ddd, J=1.0, 1.9, 4.8 Hz, 1H).

MS, APCl (%): 329 (M+1, 100), 221 (20).

### Preparation R

### trans-(2-Benzyl-1,2,3,4-tetrahydro-naphthalen-1-yl)-thien-2-ylmethyl-amine

In a procedure similar to preparation C, the product of preparation B (1.5 g, 6.3 mmol) was reacted with 2-thiophenecarboxaldehyde (0.65 mL, 6.95 mmol) to give 0.90 g of the title compound as an oil:

¹H-NMR (CDCl₃, δ): 1.6 (m, 1H), 2.1 (m, 1H), 2.3 (m, 1H), 2.46 (dd, J=8.5, 14 Hz, 1H), 2.70 (dd, J=6.8, 14 Hz, 1H), 2.7 (m, 2H), 3.60 (d, J=4.4 Hz), 3.89 (s, 2H), 6.72 (dd, J=1.1, 2.1, 1H), 6.87 (dd, J=3.4, 5.2 Hz, 1H), 7.0-7.3 (m, 11H).

MS, APCl (%): 334 (M+1, 100), 221 (80).

### Preparation S

### trans-(2-Benzyl-1,2,3,4-tetrahydro-naphthalen-1-yl)-thiazol-2-ylmethyl-amine

In a procedure similar to preparation C, the product of preparation B (1.5 g, 6.3 mmol) was reacted with 2-thiazolecarboxaldehyde (0.615 mL, 6.95 mmol) to give 0.90 g of the title compound as an oil:

¹H-NMR (CDCl₃, δ): 1.6 (m, 1H), 2.1 (m, 1H), 2.3 (m, 1H), 2.46 (dd, J=8.7, 13 Hz, 1H), 2.7 (m, 3H), 3.66 (d, J=4.6 Hz), 4.02 (d, J=15 Hz, 1H), 4.10 (d, J=15 Hz, 1H), 7.0-7.4 (m, 10H), 7.67 (d, J=3.3 Hz, 1H).

MS, APCl (%): 335 (M+1, 100), 221 (60).

### Preparation T

### trans-1-Benzylamino-2-benzylcyclohexane

In a series of procedures similar to preparations A and B, 2-benzyl cyclohexanone was converted into 1-amino-2-benzylcyclohexane in 65% via the intermediate oxime. This material (3.05 g, 16.1 mmol) was then converted to the title compound (2.05 g) in a procedure similar to preparation C, isolated as an oil:

¹H-NMR (CDCl₃, δ): 0.8-2.3 (multiplets, 11H), 3.24 (dd, J=4.0, 13 Hz, 1H), 3.72 (d, J=13 Hz, 1H), 3.94 (d, J=13 Hz, 1H), 7.1-7.5 (m, 10H).

MS, APCl (%): 280 (M+1, 100).

### Preparation U

### trans-(2-Benzyl-1,2,3,4-tetrahydro-naphthalen-1-yl)-ethyl-amine

To a 0° C. solution of the product of preparation B (2.00 g, 8.43 mmol), diisopropylethylamine (1.61 g, 9.27 mmol), and ether (50 mL) was added acetyl chloride (0.66 mL, 9.27 mmol) in a dropwise fashion. After 15 min the reaction was poured into water, extracted with ether (2×300 mL), and the combined organic layers washed with aqueous NaHCO3 (2×200 mL), ), brine, dried (Na₂SO₄), and concentrated to give a solid (2.27 g).

A solution of all of this solid and THF (20 mL) was added dropwise to a suspension of lithium aluminum hydride (0.617 g, 16.3 mmol) in THF (80 mL). After heating to reflux overnight, the reaction was cooled to −70° C. and solid sodium sulfate decahydrate was added. Following warming to room temperature, the reaction mixture was filtered and concentrated to give an oil. Chromatography on silica gel utilizing ethyl acetate/hexanes gave the title compound (0.50 g) as an oil:

¹H-NMR (CDCl₃, δ): 1.03 (t, J=7.1 Hz, 1H), 1.5.(t, 1H), 2.0 (t, 1H), 2.3 (t, 1H), 2.45 (dd, J=8.6, 14 Hz, 1H), 2.5-2.8 (multiplets, 5H), 3.52 (d, J=3.9 Hz, 1H), 7.1-7.3 (m, 9H).

MS, APCl (%): 266 (M+1, 90), 221 (100).

### Preparation V

### trans-1-Benzylamino-2-(2-methoxy-benzyl)-tetralin

In a procedure analogous to preparation A, 2-(2-Methoxy-benzyl)-1-tetralone (prepared in a procedure analogous to Org. Prep. Proc. Inter., 2, 37 (1970)), was converted to 2-(2-Methoxy-benzyl)-3,4-dihydro-2H-naphthalen-1-one oxime in 78% yield.

¹H-NMR (CDCl₃, δ): 1.8 (m, 2H), 2.65 (dt, J=4.0, 17 Hz, 1H), 2.85 (dd, J=10.5, 13 Hz, 1H), 2.96 (dd, J=5.0, 13 Hz, 1H), 3.15 (m, 1H), 3.82 (s, 3H), 3.9 (m, 1H), 6.87 (dd, J=0.8, 8.0 Hz, 1H), 6.91 (dt, J=1.1, 7.3 Hz, 1H), 7.1-7.3 (m,5H), 7.95 (dd, J=1.1, 8.0 Hz, 1H).

MS, APCl (%): 282 (M+1, 100), 264 (60).

This was then treated as described in preparation B to give 1-amino-2-(2-methoxy-benzyl)-tetralin isolated as in oil in 60% yield.

¹H-NMR (CDCl₃, δ): 1.4-2.1 (m, 3H), 2.5-2.9 (multiplets, 4H), 3.68 (d, J=6.2 Hz, 1H), 3.74 (d, J=3.5 Hz, 1H), 3.78, 3.80 (s, 3H), 6.9 (m, 2H), 7.0-7.2 (m, 6H).

MS, APCl (%): 268 (M+1, 10), 251 (100).

The title compound, an oil, was prepared in a procedure similar to preparation C in 28% yield, following silica gel chromatography.

¹H-NMR (CDCl₃, δ): 1.5 (m, 2H), 2.10 (m, 1H), 2.4 (m, 1H), 2.44 (dd, J=8.2, 13 Hz, 1H), 2.7-2.9 (m, 3H), 3.59 (d, J=5.0 Hz, 1H), 3.66 (d, J=13 Hz, 1H), 3.71 (d, J=13 Hz, 1H), 3.74 (s, 3H), 7.0-7.4 (m, 13H).

MS, APCl (%): 358 (M+1, 100), 251 (80).

### Preparation W

### trans-1-Benzylamino-2-(2-fluoro-benzyl)-tetralin

In a procedure analogous to preparation A, 2-(2-fluoro-benzyl)-1-tetralone (prepared in a procedure analogous to Org. Prep. Proc. Inter., 2, 37 (1970)), was converted to 2-(2-fluoro-benzyl)-3,4-dihydro-2H-naphthalen-1-one oxime in 98% yield. ¹H-NMR (CDCl₃, δ): 1.8 (m, 2H), 2.6-32 (multiplets, 4H), 3.9 (m, 1H), 7.1-7.4 (m, 7H), 7.94 (dd, J=1.1, 8.0 Hz, 1H).

MS, APCl (%): 270 (M+1, 100).

This was then treated as described in preparation B to give 1-amino-2-(2-fluoro-benzyl)-tetralin isolated as in oil in 56% yield.

¹H-NMR (CDCl₃, δ): 1.4-2.1 (m, 3H), 2.5-3.0 (multiplets, 4H), 3.69 (d, J=5.8 Hz, 1H), 3.79 (d, J=4.5 Hz, 1H), 7.0-7.2 (m, 8H).

MS, APCl (%): 255 (M(—NH₃)+1, 20), 221 (100).

The title compound, an oil, was prepared in a procedure similar to preparation C in 33% yield, following silica gel chromatography.

¹H-NMR (CDCl₃, δ): 1.5 (m, 2H), 2.10 (m, 1H), 2.4 (m, 1H), 2.47 (dd, J=8.3 , 13 Hz, 1H), 2.68 (dd, J=6.7, 13 Hz, 1H), 2.7 (m, 2H), 3.57 (d, J=4.2 Hz, 1H), 3.66 (d, J=13 Hz, 1H), 3.73 (d, J=13 Hz,1H), 7.0-7.3 (m, 13H).

MS, APCl (%): 346 (M+1, 10), 328 (100), 221 (100).

### Preparation X

In a procedure similar to preparation D, the following compounds were prepared and isolated:

cis-N-(Furan-2-ylmethyl)-N-(2-benzyl-1,2,3,4-tetrahydro-naphthalen-1-yl)-2-(1,3-dioxo-1,3-dihydro-isoindol-2-yl)-acetamide: MS (FAB, %): 505 (M+1, 23).

trans-N-(Furan-2-ylmethyl)-N-(2-benzyl-1,2,3,4-tetrahydro-naphthalen-1-yl)-2-(1,3-dioxo-1,3-dihydro-isoindol-2-yl)-acetamide: MS (FAB, %): 505 (M+1, 19).

trans-N-(isoxazol-5-ylmethyl)-N-(2-benzyl-1,2,3,4-tetrahydro-naphthalen-1-yl)-2-(1,3-dioxo-1,3-dihydro-isoindol-2-yl)-acetamide: MS (FAB, %): 506 (M+1, 5).

trans-N-(4-Fluoro-benzyl)-N-(2-benzyl-1,2,3,4-tetrahydro-naphthalen-1-yl)-2-(1,3-dioxo-1,3-dihydro-isoindol-2-yl)-acetamide: MS, APCl (%): 533 (M+1, 100), 313 (30).

trans-N-(2-Methoxy-benzyl )-N-(2-benzyl-1,2,3,4-tetrahydro-naphthalen-1-yl)-2-(1,3-dioxo-1,3-dihydro-isoindol-2-yl)-acetamide: MS, APCl (%): 545 (M+1, 100), 325 (10).

trans-N-(Pyridin-2-ylmethyl)-N-(2-benzyl-1,2,3,4-tetrahydro-naphthalen-1-yl)-2-(1,3-dioxo-1,3-dihydro-isoindol-2-yl)-acetamide: MS, APCl (%): 516 (M+1, 100).

trans-N-(Thien-2-ylmethyl)-N-(2-benzyl-1,2,3,4-tetrahydro-naphthalen-1-yl)-2-(1,3-dioxo-1,3-dihydro-isoindol-2-yl)-acetamide: MS, APCl (%): 521 (M+1, 100), 301 (25), 289 (30).

trans-N-Benzyl-N-(2-benzyl-cyclohexyl)-2-(1,3-dioxo-1,3-dihydro-isoindol-2-yl)-acetamide: MS, APCl (%): 467 (M+1, 100), 169 (15).

trans-N-(Pyridin-4-ylmethyl)-N-(2-benzyl-1,2,3,4-tetrahydro-naphthalen-1-yl)-2-(1,3-dioxo-1,3-dihydro-isoindol-2-yl)-acetamide: MS, FAB (%): 516 (M+1, 100).

trans-N-Ethyl-N-(2-benzyl-1,2,3,4-tetrahydro-naphthalen-1-yl)-2-(1,3-dixo-1,3-dihydro-isoindol-2-yl)-acetamide: MS, APCl (%): 453 (M+1,100), 169 (50).

trans-N-(Thiazol -2-ylmethyl)-N-(2-benzyl-1,2,3,4-tetrahydro-naphthalen-1-yl)-2-(1,3-dioxo-1,3-dihydro-isoindol-2-yl)-acetamide: MS, APCl (%): 522 (M+1, 60), 169(100).

trans-N-(Benzyl)-N-(2-[2-methoxy-benzyl]-1,2,3,4-tetrahydro-naphthalen-1-yl)-2-(1,3-dioxo-1,3-dihydro-isoindol-2-yl)-acetamide: MS, APCl (%): 545 (M+1, 100), 295 (45), 169 (45).

trans-N-(Benzyl)-N-(2-[2-fluoro-benzyl]-1,2,3,4-tetrahydro-naphthalen-1-yl)-2-(1,3-dioxo-1,3-dihydro-isoindol-2-yl)-acetamide: MS, APCl (%): 533 (M+1, 20), 515 (100), 295 (60).

### Preparation Y

In a procedure similar to E, the following compounds were prepared and isolated:

cis-2-Amino-N-(furan-2-ylmethyl)-N-(2-benzyl-1,2,3,4-tetrahydro-naphthalen-1-yl)-acetamide: MS (FAB, %): 375 (M+1 12).

trans-2-Amino-N-(furan-2-ylmethyl)-N-(Z-benzyl-1,2,3,4-tetrahydro-naphthalen-1-yl-acetamide: MS (FAB, %): 375(M+1, 22).

trans-2-Amino-N-(isoxazol-5-ylmethyl)-N-(2-benzyl-1,2,3,4-tetrahydro-naphthalen-1-yl)-acetamide.

trans-2-Amino-N-(4-fluoro-benzyl)-N-(2-benzyl-1,2,3,4-tetrahydro-naphthalen-1-yl)-acetamide: MS, APCl (%): 403 (M+1, 100), 224 (55).

trans-2-Amino-N-(2-methoxy-benzyl)-N-(2-benzyl-1,2,3,4-tetrahydro-naphthalen-1-yl)-acetamide: MS, APCl (%): 415 (M+1, 95), 195 (100).

trans-2-Amino-N-(pyridin-2-ylmethyl)-N-(2-benzyl-1,2,3,4-tetrahydro-naphthalen-1-yl)-acetamide: MS, APCl (%): 386 (M+1, 100) 166 (35).

trans-2-Amino-N-(thien-2-ylmethyl)-N-(2-benzyl-1,2,3,4-tetrahydro-naphthalen-1-yl)-acetamide: MS, APCl (%): 391 (M+1, 100), 212 (50).

trans-2-Amino-N-benzyl-N-(2-benzyl-cyclohexyl)-acetamide: MS, APCl (%): 337 (M+1, 100).

trans-2-Amino-N-(pyridin-4-ylmethyl)-N-(2-benzyl-1,2,3,4-tetrahydro-naphthalen-1-yl)-acetamide.

trans-2-Amino-N-ethyl-N-(2-benzyl-1,2,3,4-tetrahydro-naphthalen-1-yl)-acetamide: no data collected

trans-2-Amino-N-(thiazol-2-ylmethyl)-N-(2-benzyl-1,2,3,4-tetrahydro-naphthalen-1-yl)-acetamide: no data collected

trans-2-Amino-N-(benzyl)-N-(2-[2-methoxy-benzyl]-1,2,3,4-tetrahydro-naphthalen-1-yl)-acetamide: MS, APCl (%): 415 (M+1, 70), 206 (100).

trans-2-Amino-N-(benzyl)-N-(2-[2-fluoro-benzyl]-1,2,3,4-tetrahydro-naphthalen-1-yl)-acetamide: MS, APCl (%): 403 (M+1, 10), 385 (50), 206 (100).

### Preparation Z

In a procedure similar to F, the following compounds were prepared and isolated:

cis-3-(3-{[(Furan-2-ylmethyl)-(2-benzyl-1,2,3,4-tetrahydro-naphthalen-1-yl)-carbamoyl]-methyl}-ureido)-benzoic acid methyl ester: MS (FAB, %): 552 (M+1, 5).

trans-3-(3-{[(Furan-2-ylmethyl)-(2-benzyl-1,2,3,4-tetrahydro-naphthalen-1-yl)-carbamoyl]-methyl}-ureido)-benzoic acid methyl ester: MS (FAB; %): 552 (M+1, 7).

trans-3-(3-{[(Isoxazol-5-ylmethyl)-(2-benzyl-1,2,3,4-tetrahydro-naphthalen-1-yl)-carbamoyl]-methyl}-ureido)-benzoic acid methyl ester: MS (FAB, %): 553 (M+1, 10), 333 (40), 156 (65).

trans-3-(3-{[(4-Fluoro-benzyl)-(2-benzyl-1,2,3,4-tetrahydro-naphthalen-1-yl)-carbamoyl]-methyl}-ureido)-benzoic acid methyl ester: MS (APCl, %): 580 (M+1, 5), 360 (40), 183 (100).

trans-3-(3-{[(2-Methoxy-benzyl)-(2-benzyl-1,2,3,4-tetrahydro-naphthalen-1-yl)-carbamoyl]-methyl}-ureido)-benzoic acid methyl ester: MS (APCl, %): 592 (M+1, 5), 386 (30), 372 (65).

trans-3-(3-{[(Pyridin-2-ylmethyl)-(2-benzyl-1,2,3,4-tetrahydro-naphthalen-1-yl)-carbamoyl]-methyl}-ureido)-benzoic acid methyl ester: MS (APCl, %): 563 (M+1, 60), 166 (100).

trans-3-(3-{[(Thien-2-ylmethyl)-(2-benzyl-1,2,3,4-tetrahydro-naphthalen-1-yl)-carbamoyl]-methyl}-ureido)-benzoic acid methyl ester: MS (APCl, %): 568 (M+1, 85), 348 (100).

trans-3-(3-{[Benzyl-(2-benzyl-cyclohexyl)-carbamoyl]-methyl}-ureido)-benzoic acid methyl ester: MS (APCl, %): 514 (M+1, 100), 337 (80).

trans-3-(3-{[(Pyridin4-ylmethyl)-(2-benzyl-1,2,3,4-tetrahydro-naphthalen-1-yl)-carbamoyl]-methyl}-ureido)-benzoic acid methyl ester: MS (FAB, %): 563 (M+1, 25), 548 (40), 163 (30).

trans-3-(3-{[Ethyl-(2-benzyl-1,2,3,4-tetrahydro-naphthalen-1-yl)-carbamoyl]-methyl}-ureido)-benzoic acid methyl ester: MS (APCl, %): 500 (M+1, 40), 280(100), 169 (90).

trans-3-(3-{[(Thiazol-2-ylmethyl)-(2-benzyl-1,2,3,4-tetrahydro-naphthalen-1-yl)-carbamoyl]-methyl}-ureido)-benzoic acid methyl ester: MS (APCl, %): 569 (M+1, 90), 169 (100).

trans-3-(3-{[Benzyl-(2-{2-methoxy-benzyl}-1,2,3,4-tetrahydro-naphthalen-1-yl)-carbamoyl]-methyl}-ureido)-benzoic acid methyl ester: MS (APCl, %): 592 (M+1, 40), 342 (100), 206 (60).

trans-3-(3-{[Benzyl-(2-{2-fluoro-benzyl}-1,2,3,4-tetrahydro-naphthalen-1-yl)-carbamoyl]-methyl}-ureido)-benzoic acid methyl ester: MS (APCl, %): 580 (M+1, 5), 562 (40), 342 (100).

### EXAMPLE 7

### cis-3-(3-{[(2-Benzyl-1,2,3,4-tetrahydro-naphthalen-1-yl)-furan-2-ylmethyl-carbamoyl]-methyl}-ureido)-benzoic Acid

In a procedure similar to example 1, the title compound was isolated in 92% yield as an amorphous solid.

MS (FAB, %): 538 (M+1, 34).

| | C | H | N |
|---|---|---|---|
| Calc'd for C₃₂H₃₁N₃O₅ | 71.49 | 5.81 | 7.82 |
| found | 71.29 | 6.19 | 7.46 |

### EXAMPLE 8

### trans-3-(3-{[(2-Benzyl-1,2,3,4-tetrahydro-naphthalen-1-yl)-furan-2-ylmethyl-carbamoyl]-methyl}-ureido)-benzoic acid

In a procedure similar to example 1, the title compound was isolated in 97% yield as an amorphous solid.

MS (FAB, %): 538 (M+1, 22).

| | C | H | N |
|---|---|---|---|
| Calc'd for C₃₂H₃₁N₃O₅ | 71.49 | 5.81 | 7.82 |
| found | 71.28 | 6.21 | 7.55 |

### EXAMPLE 9

### trans-3-(3-{[(2-Benzyl-1,2,3,4-tetrahydro-naphthalen-1-yl)-isoxazol-5-ylmethyl-carbamoyl]-methyl}-ureido)-benzoic acid

In a procedure similar to example 1, the title compound was isolated in quantitative yield as an amorphous solid following trituration with ethyl acetate/hexanes.

MS (FAB, %): 539 (M+1, 8).

| | C | H | N |
|---|---|---|---|
| Calc'd for C₃₁H₃₀N₄O₅·0.25 H₂O | 68.56 | 5.66 | 10.32 |
| found | 68.88 | 5.99 | 10.15 |

### EXAMPLE 10

### trans-3-(3-{[(2-Benzyl-12,3,4-tetrahydro-naphthalen-1-yl)-(4-fluoro-benzyl)-carbamoyl]-methyl}-ureido)-benzoic acid

In a procedure similar to example 1, the title compound was isolated in 60% yield as an amorphous solid following trituration with ethyl acetate/hexanes.

MS (APCl, %): 564 (M−1, 30), 175 (28), 152 (100).

### EXAMPLE 11

### trans-3-(3-{[(2-Benzyl-1,2,3,4-tetrahydro-naphthalen-1-yl)-(2-methoxy-benzyl)-carbamoyl]-methyl}-ureido)-benzoic acid

In a procedure similar to example 1, the title compound was isolated in 72% yield as a white solid following trituration with ethyl acetate/hexanes, mp 117° C.

MS (APCl, %): 576 (M−1, 10), 175 (100).

### EXAMPLE 12

### trans-3-(3-{[(2-Benzyl-1,2,3,4-tetrahydro-naphthalen-1-yl)-pyridin-2-ylmethyl-carbamoyl]-methyl}-ureido)-benzoic acid

In a procedure similar to example 1, the title compound was isolated in 62% yield as a white solid following trituration with ethyl acetate/hexanes, mp 135° C.

MS (APCl, %): 546 (M−1, 100).

### EXAMPLE 13

### trans-3-(3-{[(2-Benzyl-1,2,3,4-tetrahydro-naphthalen-1-yl)-thiophen-2-ylmethyl-carbamoyl]-methyl}-ureido)-benzoic acid

In a procedure similar to example 1, the title compound was isolated in 51% yield as a white solid following trituration with ethyl acetate/hexanes, mp 196-198° C.

MS (APCl, %): 552 (M−1, 100), 153 (60).

### EXAMPLE 14

### trans-3-(3-{[Benzyl-(2-benzyl-cyclohexyl)-carbamoyl]-methyl)-ureido)-benzoic Acid

In a procedure similar to example 1, the title compound was isolated in 34% yield as a white solid following recrystallization from ethyl acetatelcyclohexanes, mp 160-162° C.

MS (APCl, %): 498 (M−1, 100), 367 (80).

### EXAMPLE 15

### trans-3-(3-{[(2-Benzyl-1,2,3,4-tetrahydro-naphthalen-1-yl)-pyridin-4-ylmethyl-carbamoyl]-methyl}-ureido)-benzoic Acid

In a procedure similar to example 1, the title compound was isolated in 80% yield as an amorphous white solid.

MS (FAB, %): 550 (M+1, 15), 534 (30), 314 (20), 119 (40).

### EXAMPLE 16

### trans-3-(3-{[(2-Benzyl-1,2,3,4-tetrahydro-naphthalen-1yl)-ethyl-carbamoyl]-methyl}-ureido)-benzoic acid

In a procedure similar to example 1, the title compound was isolated in 69% yield as a white solid following trituration with ethyl acetate/hexanes, mp 130° C.

MS (APCl, %): 484 (M−1, 100), 175 (50).

### EXAMPLE 17

### trans-3-(3-{[(2-Benzyl-1,2,3,4-tetrahydro-naphthalen-1-yl)-thiazol-2-ylmethyl-carbamoyl]-methyl}-ureido)-benzoic acid

In a procedure similar to example 1, the title compound was isolated in 46% yield as a white solid following trituration with ethyl acetate/hexanes, mp 194-195° C.

MS (APCl, %): 553 (M−1, 100), 249 (40).

### EXAMPLE 18

### trans-3-[3-({Benzyl-[2-(2-methoxy-benzyl)-1,2,3,4-tetrahydro-naphthalen-1-yl]-carbamoyl}-methyl)-ureido]-benzoic acid

In a procedure similar to example 1, the title compound was isolated in 76% yield as a white solid following trituration with ethyl acetate/hexanes, mp 130° C.

MS (APCl, %): 576 (M−1, 30), 175 (100).

### EXAMPLE 19

### trans-3-[3-({Benzyl-[2-(2-fluoro-benzyl)-1,2,3,4-tetrahydro-naphthalen-1-yl]-carbamoyl}-methyl)-ureido]-benzoic acid

In a procedure similar to example 1, the title compound was isolated in 82% yield as a white solid following trituration with ethyl acetate/hexanes, mp 150° C.

MS (APCl, %): 546 (M−1, 40), 175 (100).

### Preparation AA

### trans-6-({[Benzyl-(2-benzyl-cyclohexyl)-carbamoyl]-methyl}-carbamoyl)-pyrimidine-4-carboxylic acid methyl ester

To a solution of the product of preparation T (92 mg, 0.27 mmol), diisopropylethyl amine (0.047 mL, 0.27 mmol), pyrimidine-4,6-dicarboxylic acid monomethyl ester (50 mg, 0.27 mmol), methylene chloride (2 mL) and HOBt (10 mg) was added 1-(3-dimethylaminopropyl)-3-ethyl-carbodiimide hydrochloride (58 mg, 0.30 mmol). After stirring overnight, the reaction was poured into ethyl acetate (40 mL) and washed with 0.1 N HCl (2×20 mL), aqueous sodium bicarbonate, water, and brine, and dried over magnesium sulfate. After evaporation, the residue was chromatographed on silica gel utilizing ethyl acetate/hexane to give (51% yield) the title compound which was isolated as an oil.

MS (APCl, %): 576 (M+1, 100).

### EXAMPLE 20

### trans-6-({[Benzyl-(2-benzyl-cyclohexyl)-carbamoyl]-methyl}-carbamoyl)-pyrimidine-4-carboxylic acid

In a procedure similar to example 1, utilizing the product of preparation AA as starting material, the title compound was isolated in 72% yield as a white solid following recrystallization from isopropyl ether, mp 120° C.

MS (APCl, %): 485 (M−1, 100), 367 (40).

### Preparation AB

### trans-{1-[Benzyl-(2-benzyl-1,2,3,4-tetrahydro-naphthalen-1-yl)-carbamoyl]-2-phenyl-ethyl}-carbamic acid tert-butyl ester

To a solution of the product of preparation C (trans, 159 mg, 0.49 mmol), diisopropylethyl amine (0.25 mL, 1.5 mmol), BOC-L-Phe (142 mg, 0.54 mmol) and methylene chloride (2 mL) was added tetramethylfluoroformamidinium hexfluorophosphate (TFFH, 144 mg, 0.59 mmol) and the reaction allowed to stir at room temperature. After 5 days, an additional portion of BOC-L-Phe (71 mg, 0.27 mmol) and TFFH (172 mg, 0.30 mmol) was added. After stirring over night, the reaction was poured into ethyl acetate (30 mL) and washed with 0.1 N HCl (20 mL), aqueous sodium bicarbonate, water, and brine, and dried over magnesium sulfate. After evaporation, the residue was chromatographed on silica gel utilizing ethyl acetateihexane to give (82% yield) the title compound which was isolated as an oil.

MS (APCl, %): 475 (M+1, 30), 519 (30), 475(40), 355(70), 299 (80), 255 (100).

### Preparation AC

### trans-4-(3-{1-[Benzyl-(2-benzyl-1,2,3,4-tetrahydronaphthalen-1-yl)-carbamoyl]-2-phenyl-ethyl}-ureido)-phthalic acid dimethyl ester

To a solution of 4-amino-phthalic acid dimethyl ester (200 mg, 0.95 mmol), triethylamine (0.31 mL, 2.2 mmol) and methylene chloride (4.5 mL) was added triphosgene (97 mg, 0.32 mmol). The solution was allowed to stir for 1 h.

A solution of the product of preparation AB (230 mg, 0.39 mmol) and methylene chloride (2 mL) was cooled in an ice bath and then diluted with trifluoroacetic acid (2 mL). After stirring for 30 minutes, the solution was concentrated under vacuum. The reaction was diluted with toluene (1 mL) and concentrated two times. One half of the above prepared isocyanate solution Was added, along with triethylamine (0.31 mL, 2.2 mmol), and the reaction stirred at room temperature overnight. The reaction was poured into ethyl acetate (50 mL) and washed with 0.1 N HCl (20 mL), aqueous sodium bicarbonate, water, and. brine, and dried over magnesium sulfate. After evaporation, the residue was chromatographed on silica gel utilizing ethyl acetateihexane to give (73% yield) the title compound which was isolated as an oil.

MS (APCl, %): 710 (M+1, 15) 490 (100), 475 (10), 383 (15), 255 (20).

### EXAMPLE 21

### trans-4-(3-(1-[Benzyl-(2-benzyl-1,2,3,4-tetrahydro-naphthalen-1-yl)-carbamoyl]-2-phenyl-ethyl}-ureido)-phthalic Acid

In a procedure similar to example 1, utilizing the product of preparation AC as starting material, the title compound was isolated in 62% yield as tan foam.

MS (APCl, %): 680 (M−1, 100), 476 (30).

### Preparation AD

### trans-4-[3-Benzyl-3-(2-benzyl-1,2,3,4-tetrahydro-naphthalen-1-yl)-ureido]-phthalic acid dimethyl ester

To a solution of the product of preparation C (trans, 150 mg, 0.46 mmol) in methylene chloride (1 mL) was added 2.5 mL of the isocyanate solution from preparation AC. After stirring overnight, the reaction was poured into ethyl acetate (59 mL) and washed with 0.1 N HCl (20 mL), aqueous sodium bicarbonate, water, and brine, and dried over magnesium sulfate. After evaporation, the residue was chromatographed on silica gel utilizing ethyl acetate/hexane to give (79% yield) the title compound which was isolated as an oil.

MS (APCl, %): 563 (M+1, 100), 328 (30), 311 (30), 221.(30).

### EXAMPLE 22

### trans-4-[3-Benzyl-3-(2-benzyl-1,2,3,4-tetrahydro-naphthalen-1-yl)-ureido]-phthalic acid

In a procedure similar to example 1, utilizing the product of preparation AD as starting material, the title compound was isolated in 68% yield as tan solid following trituration with hexanes.

MS (APCl, %): 635 (M+1, 95), 328 (90), 221 (100).

### Preparation AE

### 3-Benzyl-chroman-4-ol

3-Benzyl-chroman-4-one (476 mg) is dissolved in 13.2 ml 10:1 methanol:THF and cooled to 0° C. for 10 minutes. The solution is treated with 1.5 equiv Cerium trichloride at 0° C. After 15 minutes, 2.5 equiv polymer-bound borohydride (Aldrich, 2.5 mmole/g) is added portionwise. After the addition is complete, the reaction is stirred under nitrogen at 0° C. four hours. The solids are then filtered off and washed with methanol and methylene chloride. Combined filtrates are evaporated to semisolid residues and partitioned between 10% aqueous citric acid and methylene chloride. Extracted 3 x 10 ml methylene chloride. Combined extracts are washed 1×20 ml brine, dried over sodium sulfate and evaporated to afford 394 mg 3-Benzyl-chroman-4-ol as a pink solid, 82%.

TLC (4:1 Hexanes:EtOAc) R_{f}=0.20;

Also prepared were by this method were:

### 2-Benzyl-7-methoxy-1,2,3,4-tetrahydro-naphthalen-1 -ol

2-Benzyl-7-methoxy-3,4-dihydro-2H-naphthalen-1-one (798 mg) is treated as above to afford 759 mg 2-Benzyl-7-methoxy-1,2,3,4-tetrahydro-naphthalen-1-ol as a pink oil, 94%.

### 2-Benzyl-indan-1-ol

2-Benzyl-indan-1-one (667 mg) is treated as above to afford 509 mg 2-Benzyl-indan-1-ol as a pink solid, 76%.

### 2-Benzyl-5,7-dimethyl-1,2,3,4-tetrahydro-naphthalen-1-ol

2-Benzyl-5,7-dimethyl-3,4-dihydro-2H-naphthalen-1-one (792 mg) is treated as above to afford 520 mg 2-Benzyl-5,7-dimethyl-1,2,3,4-tetrahydro-naphthalen-1-ol as a yellow oil, 65%.

### 2-Benzyl-6,7-dimethoxy-1,2,3,4-tetrahydro-naphthalen-1-ol

2-Benzyl-6,7-dimethoxy-3,4-dihydro-2H-naphthalen-1-one (888 :mg) is treated as above to afford 817 mg 2-Benzyl-6,7-dimethoxy-1,2,3,4-tetrahydro-naphthalen-1-ol as a purple solid, 91%.

### 2-(2-Methyl-benzyl)-1,2,3,4-tetrahydro-naphthalen-1-ol

2-(2-Methyl-benzyl)-3,4-dihydro-2H-naphthalen-1-one (751 mg) is treated as above to afford 623 mg 2-(2-Methyl-benzyl)-1,2,3,4-tetrahydro-naphthalen-1-ol as a colorless oil, 82%.

### 2-(3-Fluoro-benzyl)-1,2,3,4-tetrahydro,-ndphthalen-1-ol

2-(3-Fluoro-benzyl)-3,4-dihydro-2H-naphthalen-1-one (763 mg) is treated as above to afford 687 mg 2-(3-Fluoro-benzyl)-1,2,3,4-tetrahydro-naphthalen-1-ol as a colorless oil, 89%.

### 2-Benzo[1,3]dioxol-5-ylmethyl-1,2,3,4-tetrahydro-naphthalen-1-ol

2-Benzo[1,3]dioxol-5-ylmethyl-3,4-dihydro-2H-naphthalen-1-one (841 mg) is treated as above to afford 692 mg 2-Benzo[1,3]dioxol-5-ylmethyl-1,2,3,4-tetrahydro-naphthalen-1-ol as a colorless oil, 82%.

### 2-(4-Fluoro-benzyl)-1,2,3,4-tetrahydro-naphthalen-1-ol

2-(4-Fluoro-benzyl)-3,4-dihydro-2H-naphthalen-1-one (763 mg) is treated as above to afford 646 mg 2-(4-Fluoro-benzyl)-1,2,3,4-tetrahydro-naphthalen-1-ol as a colorless oil, 84%.

### 2-(4-Methoxy-benzyl)-1,2,3,4-tetrahydro-naphthalen-1-ol

2-(4-Methoxy-benzyl)-3,4-dihydro-2H-naphthalen-1-one (799 mg) is treated as above to afford 628 mg 2-(4-Methoxy-benzyl)-1,2,3,4-tetrahydro-naphthalen-1-ol as a colorless oil, 78%.

### 2-(4-Chloro-benzyl)-1,2,3,4-tetrahydro-naphthalen-1-ol

2-(4-Chloro-benzyl)-3,4-dihydro-2H-naphthalen-1-one (812 mg) is treated as above to afford 701 mg 2-(4-Chloro-benzyl)-1,2,3,4-tetrahydro-naphthalen-1-ol as a colorless oil, 86%.

### 2-(2-Chloro-benzyl)-1,2,3,4-tetrahydro-naphthalen-1-ol

2-(2-Chloro-benzyl)-3,4-dihydro-2H-naphthalen-1-one (812 mg) is treated as above to afford 872 mg colorless oil, which is re-treated to afford 225 mg 2-(2-Chloro-benzyl)-1,2,3,4-tetrahydro-naphthalen-1-ol as a pink oil, 27%.

### 2-Benzyl-6-methoxy-1,2,3,4-tetrahydro-naphthalen-1-ol

2-Benzyl-6-methoxy-3,4-dihydro-2H-naphthalen-1-one is dissolved in 13.2 ml 10:1 methanol:THF and cooled to 0° C. for 10 minutes. The solution is treated with 1.5.equiv Cerium trichloride at 0° C. After 15 minutes, 2.5 equiv copolymer-bound borohydride (Aldrich, 2.5 mmole/g) is added portionwise. After the addition is complete, the reaction is stirred under nitrogen at 0° C. four hours. The solids are then filtered off and washed with methanol and methylene chloride. Combined filtrates are evaporated to semisolid residues and partitioned between 10% aqueous citric acid and methylene, chloride. Extracted 3×10 ml methylene chloride. Combined extracts are washed 1×20 ml brine, dried. over sodium sulfate and evaporated to afford 248 mg orange oil. The oil is chromatographed on 50 cc silica gel, eluting with 20% ethyl acetate/hexanes. Product fractions are evaporated to afford 263mg colorless oil, 49%.

### 2-(4-Methyl-benzyl)-1,2,3,4-tetrahydro-naphthalen-1-ol

2-(4-Methyl-benzyl)-3,4-dihydro-2H-naphthalen-1-one (751 mg) is suspended in 10:1 methanol:THF and cooled to 0° C. for 10 minutes. The solution is treated with 1.5 equiv Cerium trichloride at 0° C. After 15 minutes, 2.5 equiv polymer-bound borohydride (Aldrich, 2.5 mmole/g) is added portionwise. After the addition is complete, the reaction is stirred under nitrogen at 0° C. four hours. The solids are then filtered off and washed with methylene chloride. The solvent is evaporated to give 780 mg 2-(4-Methyl-benzyl)-1,2,3,4-tetrahydro-naphthalen-1-ol as a pink oil, which is used without further purification.

### 2-(3-Methyl-benzyl)-1,2,3,4-tetrahydro-naphthalen-1-ol

2-(3-Methyl-benzyl)-3,4-dihydro-2H-naphthalen-1-one (751 mg) is treated as above to afford 785 mg 2-(3-Methyl-benzyl)-1,2,3,4-tetrahydro-naphthalen-1-ol as a pink oil.

### 2-(3-Methoxy-benzyl)-1,2,3,4-tetrahydro-naphthalen-1-ol

2-(3-Methoxy-benzyl)-3,4-dihydro-2H-naphthalen-1-one (800 mg) is treated as above to afford 817 mg 2-(3-Methoxy-benzyl)-1,2,3,4-tetrahydro-naphthalen-1-ol as a pink oil.

### 2-(3,4-Dichloro-benzyl)-1,2,3,4-tetrahydro-naphthalen-1-ol

2-(3,4-Dichloro-benzyl)-3,4-dihydro-2H-naphthalen-1-one (915 mg) is treated as above to afford 872 mg 2-(3,4-Dichloro-benzyl)-1,2,3,4-tetrahydro-naphthalen-1-ol as a pink oil.

### 1,3-Diphenyl-propan-1-ol

Commercially available 1,3-diphenyl-1-propanone (840 mg) is treated as above to afford 800 mg 1,3-Diphenyl-propan-1-ol, 94%.

### Preparation AF

### 4-Azido-3-benzyl-chroman

A solution of 394 mg 3-Benzyl-chroman-4-ol in 2.46 ml 2:1 toluene:THF is treated with 1.5 equiv diphenylphosphoryl azide. and 1.5 equiv 1,8-diazabicyclo[5.4.0]undec-7-ene at ambient temperature 18 hours. The mixture is diluted with 2 ml water, stirred 5 minutes, and extracted 3×4 ml ethyl acetate. Combined extracts are washed with brine, dried over sodium sulfate, and evaporated to afford an orange oil, 570 mg. The oil is chromatographed on 50 cc silica gel, eluting with 5% ethyl acetate/hexanes. Product fractions are evaporated to give 285 mg 4-Azido-3-benzyl-chroman as a colorless oil, 65%.

TLC (4:1 Hexanes:EtOAc) R_{f}=0.55; MS, APCl+(%) 238 (M−N₂, 100%); ¹H NMR (CDCl₃) δ2.17 (m, 1H), 2.63 (d, 2H),4.00 (dd, 1H), 4.15 (dd, 1H), 4.28 (s, 1H), 6.93 (m, 2H), 7.22 (m, 7H).

Also prepared were:

### 1-Azido-2-benzyl-6-methoxy-1,2,3,4-tetrahydro-naphthalene

A solution of 263 mg 2-Benzyl-6-methoxy-1,2,3,4-tetrahydro-naphthalen-1-ol in 1.5 ml 2:1 toluene:THF is treated with 1.5 equiv diphenylphosphoryl azide and 1.5 equiv 1,8-diazabicyclo[5.4.0]undec-7-ene at ambient temperature 18 hours. The mixture is diluted with 1 ml water, stirred 5 minutes, and extracted 3×4 ml ethyl acetate. Combined extracts are washed with brine, dried over sodium sulfate, and evaporated to afford an orange oil, 417 mg. The product is used without further purification.

### 1-Azido-2-benzyl-7-methoxy-1,2,3,4-tetrahydro-naphthalene

2-Benzyl-7-methoxy-1,2,3,4-tetrahydro-naphthalen-1-ol (759 mg) is treated as above to give impure 1-Azido-2-benzyl-7-methoxy-1,2,3,4-tetrahydro-naphthalene as an amber oil, which is used without further purification.

### 1-Azido-1,3-diphenyl Propane

1,3-Diphenyl-propan-1-ol (210 mg) is treated as above to give 140 mg 1-Azido-1,3-diphenyl propane, 61%.

### 1-Azido-2-benzyl-indan

2-Benzyl-indan-1-ol (509 mg) is treated as above to give impure 1-Azido-2-benzyl-indan as an amber oil, which is used without further purification.

### 1-Azido-2-benzyl-5,7-dimethyl-1,2,3,4-tetrahydro-naphthalene

2-Benzyl-5,7-dimethyl-1,2,3,4-tetrahydro-naphthalen-1-ol (520 mg) is treated as above to give impure 1-Azido-2-benzyl-5,7-dimethyl-1,2,3,4-tetrahydro-naphthalene as an amber oil, which is used without further purification.

### 1-Azido-2-benzyl-6,7-dimethoxy-1,2,3,4-tetrahydro-naphthalene

2-Benzyl-6,7-dimethoxy-1,2,3,4-tetrahydro-naphthalen-1-ol (817 mg) is treated as above to give impure 1-Azido-2-benzyl-6,7-dimethoxy-1,2,3,4-tetrahydro-naphthalene as an amber oil, which is used without further purification.

### 1-Azido-2-(2-methyl-benzyl)-1,2,3,4-tetrahydro-naphthalene

2-(2-Methyl-benzyl)-1,2,3,4-tetrahydro-naphthalen-1-ol (623 mg) is treated as above to give impure 1-Azido-2-(2-methyl-benzyl)-1,2,3,4-tetrahydro-naphthalene as an orange oil, which is used without further purification.

### 1-Azido-2-(3-fluoro-benzyl)-1,2,3,4-tetrahydro-naphthalene

2-(3-Fluoro-benzyl)-1,2,3,4-tetrahydro-naphthalen-1-ol (687 mg) is treated as above to give 1-Azido-2-(3-fluoro-benzyl)-1,2,3,4-tetrahydro-naphthalene as an orange oil, which is used without further purification.

### 5-(1-Azido-1,2,3,4-tetrahydro-naphthalen-2-ylmethyl)-benzo[1,3]dioxole

2-Benzo[1,3]dioxol-5-ylmethyl-1,2,3,4-tetrahydro-naphthalen-1-ol (692 mg) is treated as above to give 5-(1-Azido-1,2,3,4-tetrahydro-naphthalen-2-ylmethyl)-benzo[1,3]dioxole as an orange oil, which is used without further purification.

### 1-Azido-2-(2-chloro-benzyl)-1,2,3,4-tetrahydro-naphthalene

2-(2-Chloro-benzyl)-1,2,3,4-tetrahydro-naphthalen-1-ol (225 mg) is treated as above to give 1-Azido-2-(2-chloro-benzyl)-1,2,3,4-tetrahydro-naphthalene as an orange oil, which is used without further purification.

### 1-Azido-2-(4-fluoro-benzyl)-1,2,3,4-tetrahydro-naphthalene

2-(4-Fluoro-benzyl)-1,2,3,4-tetrahydro-naphthalen-1-ol (646 mg) is treated as above to give 1-Azido-2-(4-fluoro-benzyl)-1,2,3,4-tetrahydro-naphthalene as an orange oil, which is used without further purification.

### 1-Azido-2-(4-methoxy-benzyl)-1,2,3,4-tetrahydro-naphthalene

2-(4-Methoxy-benzyl)-1,2,3,4-tetrahydro-naphthalen-1-ol (628 mg) is treated as above to give 1-Azido-2-(4-methoxy-benzyl)-1,2,3,4-tetrahydro-naphthalene as an orange oil, which is used without further purification.

### 1-Azido-2-(4-chloro-benzyl)-1,2,3,4-tetrahydro-naphthalene

2-(4-Chloro-benzyl)-1,2,3,4-tetrahydro-naphthalen-1-ol (702 mg) is treated as above to give 1-Azido-2-(4-chloro-benzyl)-1,2,3,4-tetrahydro-naphthalene as an orange oil, which is used without further purification.

### 1-Azido-2-(4-methyl-benzyl)-1,2,3,4-tetrahydro-naphthalene

2-(4-Methyl-benzyl)-1,2,3,4-tetrahydro-naphthalen-1-ol (780 mg) is treated as above to give 1-Azido-2-(4-methyl-benzyl)-1,2,3,4-tetrahydro-naphthalene as an dark oil, which is used without further purification.

### 1-Azido-2-(3-methyl-benzyl)-1,2,3,4-tetrahydro-naphthalene

2-(3-Methyl-benzyl)-1,2,3,4-tetrahydro-naphthalen-1-ol (780 mg) is treated as above to give 1-Azido-2-(3-methyl-benzyl)-1,2,3,4-tetrahydro-naphthalene as an dark oil, which is used without further purification.

### 1-Azido-2-(3-methoxy-benzyl)-1,2,3,4-tetrahydro-naphthalene

2-(3-Methoxy-benzyl)-1,2,3,4-tetrahydro-naphthalen-1-ol (817 mg) is treated as above to give 1-Azido-2-(3-methoxy-benzyl)-1,2,3,4-tetrahydro-naphthalene as an dark oil, which is used without further purification.

### 1-Azido-2-(3,4-dichloro-benzyl)-1,2,3,4-tetrahydro-naphthalene

2-(3,4-Dichloro-benzyl)-1,2,3,4-tetrahydro-naphthalen-1-ol (872 mg) is treated as above to give 1-Azido-2-(3,4-dichloro-benzyl)-1,2,3,4-tetrahydro-naphthalene as an dark oil, which is used without further purification.

### Preparation AG

### Benzyl-(3-benzyl-chroman-4-yl)-amine

A solution of 285 mg 4-Azido-3-benzyl-chroman in 8 ml methanol is treated with 0.1 equiv nickel acetate tetrahydrate and 4.1 equiv polymer-bound borohydride (Aldrich, 2.5 mmole/g) at ambient temperature for 2 hours. The mixture is filtered through a fiberglass pad, washed with methanol, and the filtrate evaporated to a green oil. The oil is dissolved in 4 ml methanol and treated with 1.6 equiv benzaldehyde and 1.1 equiv acetic acid. 4 ml methanol and 4 ml THF are added to facilitate stirring when a heavy white precipitate forms. The reaction is stirred at ambient temperature 18 hours. Polymer-bound borohydride (1.8 g, Aldrich, 2.5 mmole/g) is added portionwise. The reaction is vented and shaken at ambient temperature 3 hours. The black resin is filtered off and the filtrate evaporated to give a dark oil. The oil is dissolved in 2 ml 6:3:1 ethyl acetate:methylene chloride:methanol and divided equally between two preconditioned SCX columns (Applied Biosystems, 2 g/6 ml). The columns are washed with 6:3:1 ethyl acetate:methylene chloride:methanol and the product is released with 0.3 M ammonia/methanol solution. Solvent evaporated to afford 268 mg Benzyl-(3-benzyl-chroman-4-yl)-amine, 74%.

TLC (4:1 Hexanes:EtOAc) R_{f}=0.75; MS, APCl+(%) 330.3 (M+1, 60%), 223.2 (100%); ¹H NMR (CDCl₃) δ2.20 (m, 1H), 2.54 (m, 1H), 2.63 (m, 1H), 3.52 (s, 1H), 3.74 (d, 1H), 3.86 (d, 1H), 4.01 (dxd, 1H), 4.38 (dxd, 1H), 6.89 (d, 2H), 7.05 (d, 1H), 7.23 (m, 12H).

Also prepared were:

### Benzyl-(2-benzyl-6-methoxy-1,2,3,4-tetrahydro-naphthalen-1-yl)-amine

1-Azido-2-benzyl-6-methoxy-1,2,3,4-tetrahydro-naphthalene was treated as above to give Benzyl-(2-benzyl-6-methoxy-1,2,3,4-tetrahydro-naphthalen-1-yl)-amine, 192 mg, 55%.

### Benzyl-(2-benzyl-7-methoxy-1,2,3,4-tetrahydro-naphthalen-1-yl)-amine

1-Azido-2-benzyl-7-methoxy-1,2,3,4-tetrahydro-naphthalene was treated as above to give Benzyl-(2-benzyl-7-methoxy-1,2,3,4-tetrahydro-naphthalen-1-yl)-amine, 443 mg, 44%.

### Benzyl-(1,3-diphenyl-propyl)-amine

1-Azido-1,3-diphenyl propane was treated as above to give Benzyl-(1,3-diphenyl-propyl)-amine, 280 mg, 43%.

### Benzyl-(2-benzyl-indan-1-yl)-amine

1-Azido-2-benzyl-indan was treated as above to give Benzyl-(2-benzyl-indan-1-yl)-amine, 40%.

### Benzyl-(2-benzyl-5,7-dimethyl-1,2,3,4-tetrahydro-naphthalen-1-yl)-amine

1-Azido-2-benzyl-5,7-dimethyl-1,2,3,4-tetrahydro-naphthalene was treated as above to give Benzyl-(2-benzyl-5,7-dimethyl-1,2,3,4-tetrahydro-naphthalen-1-yl)-amine, 26%.

### Benzyl-(2-benzyl-6,7-dimethoxy-1,2,3,4-tetrahydro-naphthalen-1-yl)-amine

1-Azido-2-benzyl-6,7-dimethoxy-1,2,3,4-tetrahydro-naphthalene was treated as above to give Benzyl-(2-benzyl-6,7-dimethoxy-1,2,3,4-tetrahydro-naphthalen-1-yl)-amine, 34%.

### Benzyl-[2-(2-chloro-benzyl)-1,2,3,4-tetrahydro-naphthalen-1-yl]-amine

1-Azido-2-(2-chloro-benzyl)-1,2,3,4-tetrahydro-naphthalene was treated as above to give Benzyl-[2-(2-chloro-benzyl)-1,2,3,4-tetrahydro-naphthalen-1-yl]-amine, 32%.

### Benzyl-[2-(2-methyl-benzyl)-1,2,3,4-tetrahydro-naphthalen-1-yl]-amine

1-Azido-2-(2-methyl-benzyl)-1,2,3,4-tetrahydro-naphthalene was treated as above to give Benzyl-[2-(2-methyl-benzyl)-1,2,3,4-tetrahydro-naphthalen-1-yl]-amine, 49%.

### Benzyl-[2-(3-fluoro-benzyl)-1,2,3,4-tetrahydro-naphthalen-1-yl]-amine

1-Azido-2-(3-fluoro-benzyl)-1,2,3,4-tetrahydro-naphthalene was treated as above to give Benzyl-[2-(3-fluoro-benzyl)-1,2,3,4-tetrahydro-naphthalen-1-yl]-amine, 54%.

### (2-Benzo[1,3]dioxol-5-ylmethyl-1,2,3,4-tetrahydro-naphthalen-1-yl)-benzyl-amine

5-(1-Azido-1,2,3,4-tetrahydro-naphthalen-2-ylmethyl)-benzo[1,3]dioxole was treated as above to give (2-Benzo[1,3]dioxol-5-ylmethyl-1,2,3,4-tetrahydro-naphthalen-1-yl)-benzyl-amine, 60%.

### Benzyl-[2-(4-chloro-benzyl)-1,2,3,4-tetrahydro-naphthalen-1-yl]-amine

1-Azido-2-(4-chloro-benzyl)-1,2,3,4-tetrahydro-naphthalene was treated as above to give Benzyl-[2-(4-chloro-benzyl)-1,2,3,4-tetrahydro-naphthalen-1-yl]-amine, 44%.

### Benzyl-[2-(4-fluoro-benzyl)-1,2,3,4-tetrahydro-naphthalen-1-yl]-amine

1-Azido-2-(4-fluoro-benzyl)-1,2,3,4-tetrahydro-naphthalene was treated as above to give Benzyl-[2-(4-fluoro-benzyl)-1,2,3,4-tetrahydro-naphthalen-1-yl]-amine, 48%.

### Benzyl-[2-(4-methoxy-benzyl)-1,2,3,4-tetrahydro-naphthalen-1-yl]-amine

1-Azido-2-(4-methoxy-benzyl)-1,2,3,4-tetrahydro-naphthalene was treated as above to give Benzyl-[2-(4-methoxy-benzyl)-1,2,3,4-tetrahydro-naphthalen-1-yl]-amine, 46%.

### Benzyl-[2-(4-methyl-benzyl)-1,2,3,4-tetrahydro-naphthalen-1-yl]-amine

1-Azido-2-(4-methyl-benzyl)-1,2,3,4-tetrahydro-naphthalene was treated as above to give Benzyl-[2-(4-methylbenzyl)-1,2,3,4-tetrahydro-naphthalen-1-yl]-amine, 38%.

### Benzyl-[2-(3-methyl-benzyl)-1,2,3,4-tetrahydro-naphthalen-1-yl]-amine

1-Azido-2-(3-methyl-benzyl)-1,2,3,4-tetrahydro-naphthalene was treated as above to give Benzyl-[2-(3-methyl-benzyl)-1,2,3,4-tetrahydro-naphthalen-1-yl]-amine, 54%.

### Benzyl-[2-(3-methoxy-benzyl)-1,2,3,4-tetrahydro-naphthalen-1-yl]-amine

1-Azido-2-(3-methoxy-benzyl)-1,2,3,4-tetrahydro-naphthalene was treated as above to give Benzyl-[2-(3-methoxy-benzyl)-1,2,3,4-tetrahydro-naphthalen-1-yl]-amine, 30%.

### Benzyl-[2-(3,4-dichloro-benzyl)-1,2,3,4-tetrahydro-naphthalen-1-yl]-amine

1-Azido-2-(3,4-dichloro-benzyl)-1,2,3,4-tetrahydro-naphthalene was treated as above to give Benzyl-[2-(3,4-dichloro-benzyl)-1,2,3,4-tetrahydro-naphthalen-1-yl]-amine, 42%.

### Preparation AH

### N-Benzyl-N-(3-benzyl-chroman-4-yl)-2-(1,3-dioxo-1,3-dihydro-isoindol-2-yl)-acetamide

6 mg 4-Dimethylamino pyridine and 1.25 equiv morpholinomethyl polystyrene (Fluka, 2.4 mmole/g) are added to a solution of 159 mg Benzyl-(3-benzyl-chroman-4-yl)-amine in 3 ml dichloroethane. The mixture is cooled to 0° C. before adding dropwise a solution of (1,3-Dioxo-1,3-dihydro-isoindol-2-yl)-acetyl chloride in 1.6 ml dichloroethane. The mixture is gradually warmed to ambient temperature and stirred as such 18 hours. Aminomethyl resin (1.25 equiv, Fluka, 1.1 mmole/g) is added, followed by 10 ml dichloroethane. Shaken 18 hours at ambient temperature. The resin is filtered off and washed with methylene chloride. The filtrate is evaporated to afford an amber oil. The oil is dissolved in 2 ml 15% isopropanol/chloroform and divided between two preconditioned SCX columns. (Applied Biosystems, 2 g/6 ml). The product is eluted with 15% isopropanol/chloroform. The solvent is evaporated to give 223 mg N-Benzyl-N-(3-benzyl-chroman-4-yl)-2-(1,3-dioxo-1,3-dihydro-isoindol-2-yl)-acetamide as a white foam, 53%.

HPLC*Ret time 7.79 min; MS, APCl+(%) 517.3 (M+1, 30%), 295.2 (100%).

Also prepared were:

### N-Benzyl-N-(2-benzyl-6-methoxy-1,2,3,4-tetrahydro-naphthalen-1-yl)-2-(1,3-dioxo-1,3-dihydro-isoindol-2-yl)-acetamide

Benzyl-(2-benzyl-6-methoxy-1,2,3,4-tetrahydro-naphthalen-1-yl)-amine is treated as above to, afford N-Benzyl-N-(2-benzyl-6-methoxy-1,2,3,4-tetrahydro-naphthalen-1-yl)-2-(1,3-dioxo-1,3-dihydro-isoindol-2-yl)-acetamide as a white foam, 54%.

### N-Benzyl-N-(2-benzyl-7-methoxy-1,2,3,4-tetrahydro-naphthalen-1-yl)-2-(1,3-dioxo-1,3-dihydro-isoindol-2-yl)-acetamide

Benzyl-(2-benzyl-7-methoxy-1,2,3,4-tetrahydro-naphthalen-1-yl)-amine is treated as above to afford N-Benzyl-N-(2-benzyl-7-methoxy-1,2,3,4-tetrahydro-naphthalen-1-yl)-2-(1,3-dioxo-1,3-dihydro-isoindol-2-yl)-acetamide, 46%.

### N-Benzyl-2-(1,3-dioxo-1,3-dihydro-isoindol-2-yl)-N-(1,3-diphenyl-propyl)-acetamide

Benzyl-(1,3-diphenyl-propyl)-amine is treated as above to afford N-Benzyl-2-(1,3-dioxo-1,3-dihydro-isoindol-2-yl)-N-(1,3-diphenyl-propyl)-acetamide as a white foam, 54%.

### N-Benzyl-N-(2-benzyl-indan-1-yl)-2-(1,3-dioxo-1,3-dihydro-isoindol-2-yl)-acetamide

Benzyl-(2-benzyl-indan-1-yl)-amine is treated as above to afford N-Benzyl-N-(2-benzyl-indan-1-yl)-2-(1,3-dioxo-1,3-dihydro-isoindol-2-yl)-acetamide, 46%.

### N-Benzyl-N-(2-benzyl-5,7-dimethyl-1,2,3,4-tetrahydro-naphthalen-1-yl)-2-(1,3-dioxo-1,3-dihydro-isoindol-2-yl)-acetamide

Benzyl-(2-benzyl-5,7-dimethyl-1,2,3,4-tetrahydro-naphthalen-1-yl)-amine is treated as above to afford N-Benzyl-N-(2-benzyl-5,7-dimethyl-1,2,3,4-tetrahydro-naphthalen-1-yl)-2-(1,3-dioxo-1,3-dihydro-isoindol-2-yl)-acetamide, 60%.

### N-Benzyl-N-(2-benzyl-6,7-dimethoxy-1,2,3,4-tetrahydro-naphthalen-1-yl)-2-(1,3-dioxo-1,3-dihydro-isoindol-2-yl)-acetamide

Benzyl-(2-benzyl-6,7-dimethoxy-1,2,3,4-tetrahydro-naphthalen-1-yl)-amine is treated as above to afford N-Benzyl-N-(2-benzyl-6,7-dimethoxy-1,2,3,4-tetrahydro-naphthalen-1-yl)-2-(1,3-dioxo-1,3-dihydro-isoindol-2-yl)-acetamide, 59%.

### N-Benzyl-2-(1,3-dioxo-1,3-dihydro-isoindol-2-yl)-N-[2-(4-methyl-benzyl)-1,2,3,4-tetrahydro-naphthalen-1-yl]-acetamide

Benzyl-[2-(4-methyl-benzyl)-1,2,3,4-tetrahydro-naphthalen-1-yl]-amine is treated as above to afford N-Benzyl-2-(1,3-dioxo-1,3-dihydro-isoindol-2-yl)-N-[2-(4-methyl-benzyl)-1,2,3,4-tetrahydro-naphthalen-1-yl]-acetamide as a white foam, 68%.

### N-Benzyl-2-(1,3-dioxo-1,3-dihydro-isoindol-2-yl)—N-[2-(3-methyl-benzyl)-1,2,3,4-tetrahydro-naphthalen-1-yl]-acetamide

Benzyl-[2-(3-methyl-benzyl)-1,2,3,4-tetrahydro-naphthalen-1-yl]-amine is treated as above to afford N-Benzyl-2-(1,3-dioxo-1,3-dihydro-isoindol-2-yl)-N-[2-(3-methyl-benzyl) 1,2,3,4-tetrahydro-naphthalen-1-yl]-acetamide as a white foam, 50%.

### N-Benzyl-2-(1,3-dioxo-1,3-dihydro-isoindol-2-yl)—N-[2-(3-methoxy-benzyl)-1,2,3,4-tetrahydro-naphthalen-1-yl]-acetamide

Benzyl-[2-(3-methoxy-benzyl)-1,2,3,4-tetrahydro-naphthaien-1-yl]-amine is treated as above to afford N-Benzyl-2-(1,3-dioxo-1,3-dihydro-isoindol-2-yl)-N-[2-(3-methoxy-benzyl) 1,2,3,4-tetrahydro-naphthalen-1-yl]-acetamide as a white foam, 87%.

### N-Benzyl-N-[2-(3,4-dichloro-benzyl)-1,2,3,4-tetrahydro-naphthalen-1-yl]-2-(1,3-dioxo-1,3-dihydro-isoindol-2-yl)-acetamide

Benzyl-[2-(3,4-dichloro-benzyl)-1,2,3,4-tetrahydro-naphthalen-1-yl]-amine is treated as above to afford N-Benzyl-N-[2-(3,4-dichloro-benzyl)-1,2,3,4-tetrahydro-naphthalen-1-yl]-2-(1,3-dioxo-1,3-dihydro-isoindol-2-yl)-acetamide as a white foam, 61%.

### N-Benzyl-2-(1,3-dioxo-1,3-dihydro-isoindol-2-yl)-N-[2-(2-methyl-benzyl)-1,2,3,4-tetrahydro-naphthalen-1-yl]-acetamide

Benzyl-[2-(2-methyl-benzyl)-1,2,3,4-tetrahydro-naphthalen-1-yl]-amine is treated as above to afford N-Benzyl-2-(1,3-dioxo-1,3-dihydro-isoindol-2-yl)-N-[2-(2-methyl-benzyl)-1,2,3,4-tetrahydro-naphthalen-1-yl]-acetamide as a white foam, 60%.

### N-Benzyl-2-(1,3-dioxo-1,3-dihydro-isoindol-2-yl)-N-[2-(3-fluoro-benzyl)-1,2,3,4-tetrahydro-naphthalen-1-yl]-acetamide

Benzyl-[2-(3-fluoro-benzyl)-1,2,3,4-tetrahydro-naphthalen-1-yl]-amine is treated as above to afford N-Benzyl-2-(1,3-dioxo-1,3-dihydro-isoindol-2-yl)-N-[2-(3-fluoro-benzyl-1-1,2,3,4-tetrahydro-naphthalen-1-yl]-acetamide as a white foam, 67%.

### N-(2-Benzo[1,3]dioxol-5-ylmethyl-1,2,3,4-tetrahydro-naphthalen-1-yl)-N-benzyl-2-(1,3-dioxo-1,3-dihydro-isoindol-2-yl)-acetamide

(2-Benzo[1,3]dioxol-5-ylmethyl-1,2,3,4-tetrahydro-naphthalen-1-yl)-benzyl-amine is treated as above to afford N-(2-Benzo[1,3]dioxo-5-ylmethyl-1,2,3,4-tetrahydro-naphthalen-1-yl)-N-benzyl-2-(1,3-dioxo-1,3-dihydro-isoindol-2-yl)-acetamide as a white foam, 52%.

### N-Benzyl-N-[2-(2-chloro-benzyl)-1,2,3,4-tetrahydro-naphthalen-1-yl]-2-(1,3-dioxo-1,3-dihydro-isoindol-2-yl)-acetamide

Benzyl-[2-(2-chloro-benzyl)-1,2,3,4-tetrahydro-naphthalen-1-yl]-amine is treated as above to afford N-Benzyl-N-[2-(2-chloro-benzyl)-1,2,3,4-tetrahydro-naphthalen-1-yl]-2-(1,3-dioxo-1,3-dihydro-isoindol-2-yl)-acetamide as a white foam, 73%.

### N-Benzyl-2-( 1,3-dioxo-1,3-dihydro-isoindol-2-yl)-N-[2-(4-fluoro-benzyl)-1,2,3,4-tetrahydro-naphthalen-1-yl]-acetamide

Benzyl-[2-(4-fluoro-benzyl)-1,2,3,4-tetrahydro-naphthalen-1-yl]-amine is treated as above to afford N-Benzyl-2-(1,3-dioxo-1,3-dihydro-isoindol-2-yl)-N-[2-(4-fluoro-benzyl)-1,2,3,4-tetrahydro-naphthalen-1-yl]-acetamide as a white foam, 79%.

### N-Benzyl-2-(l ,3-dioxo-1,3-dihydro-isoindol-2-yl)-N-[2-(4-methoxy-benzyl)-1 2,3,4-tetrahydro-naphthalen-1-yl]-acetamide

Benzyl-[2-(4-methoxy-benzyl)-1,2,3,4-tetrahydro-naphthalen-1-yl]-amine is treated as above to afford N-Benzyl-2-(1,3-dioxo-1,3-dihydro-isoindol-2-yl)-N-[2-(4-methoxy-benzyl)-1,2,3,4-tetrahydro-naphthalen-1-yl]-acetamide as a white foam, 67%.

### N-Benzyl-N-[2-(4-chloro-benzyl)-1,2,3,4-tetrahydro-naphthalen-1-yl]-2-(1,3-dioxo-1,3-dihydro-isoindol-2-yl)-acetamide

Benzyl-[2-(4-chloro-benzyl)-1,2,3,4-tetrahydro-naphthalen-1-yl]-amine is treated as above to afford N-Benzyl-N-[2-(4-chloro-benzyl)-1,2,3,4-tetrahydro-naphthalen-1-yl]-2-(1,3-dioxo-1,3-dihydro-isoindol-2-yl)-acetamide as a white foam, 65%.

### Preparation AI

### 2-Amino-N-benzyl-N-(3-benzyl-chroman-4-yl)-acetamide

N-Benzyl-N-(3-benzyl-chroman-4-yl)-2-(1,3-dioxo-1,3-dihydro-isoindol-2-yl)-acetamide (223 mg) is dissolved in 4.3 ml ethanol and treated with 1.5 equiv hydrazine hydrate at ambient temperature 18 hours. The -resulting white solid is filtered off and the filtrate is evaporated to a sticky white residue. The residue is dissolved in 1 ml 15% isopropanol/chloroform and applied to a preconditioned SCX column (Applied Biosystems, 2 g/6 ml). After elution with five column volumes of 15% isopropanol/chloroform, the product is released with 0.3 M ammonia/methanol solution. The solvent is evaporated to afford another sticky residue, which is taken up in methylene chloride and filtered. The filtrate is evaporated to give 101 mg 2-Amino-N-benzyl-N-(3-benzyl-chroman-4-yl)-acetamide, 61%.

MS, APCl+(%) 387.3 (M+1, 50%), 223.2 (100%).

Also prepared were:

### 2-Amino-N-benzyl-N-(2-benzyl-6-methoxy-1,2,3,4-tetrahydro-naphthalen-1-yl)-acetamide

N-Benzyl-N-(2-benzyl-6-methoxy-1,2,3,4-tetrahydro-naphthalen-1-yl)-2-(1,3-dioxo-1,3-dihydroisoindol-2-yl)-acetamide (1 59 mg) is treated as above to afford 77 mg 2-Amino-N-benzyl-N-(2-benzyl-6-methoxy-1,2,3,4-tetrahydro-naphthalen-1-yl)-acetamide, 64%.

### 2-Amino-N-benzyl-N-(1,3-diphenyl-propyl)-acetamide

N-Benzyl-2-(1,3-dioxo-1,3-dihydro-isoindol-2-yl)-N-(1,3-diphenyl-propyl)-acetamide (234 mg) is treated as above, except using 2.5 equiv hydrazine hydrate, to afford 101 mg 2-Amino-N-benzyl-N-(1,3-diphenyl-propyl)-acetamide as a white foam, 58%.

### 2-Amino-N-benzyl-N-(2-benzyl-7-methoxy-1,2,3,4-tetrahydro-naphthalen-1-yl)-acetamide

N-Benzyl-N-(2-benzyl-7-methoxy-1,2,3,4-tetrahydro-naphthalen-1-yl)-2-(1,3-dioxo-1,3-dihydro-isoindol-2-yl)-acetamide (140 mg) is dissolved in 1:1 methyl ene chloride:THF and treated with 2 equiv hydrazine hydrate in ethanol at ambient temperature 18 hours. The resulting white solid is filtered off and the filtrate is evaporated to a sticky white residue. The residue is dissolved in 15% isopropanol/chloroform and applied to a preconditioned SCX column (Applied, Biosystems, 2 g/6 ml). After elution with five column volumes of 15% isopropanol/chloroform, the product is released with 0.3 M ammonia/methanol solution. The solvent is evaporated to afford a colorless oil, 2-Amino-N-benzyl-N-(2-benzyl-7-methoxy-1,2,3,4-tetrahydro-naphthalen-1-yl)-acetamide, 97 mg, 90%.

### 2-Amino-N-benzyl-N-(2-benzyl-indan-1-yl)-acetamide

N-Benzyl-N-(2-benzyl-indan-1-yl)-2-(1,3-dioxo-1,3-dihydro-isoindol-2-yl)-acetamide (208 mg) is treated as above to afford 27 mg 2-Amino-N-benzyl-N-(2-benzyl-indan-1-yl)-acetamide, 18%.

### 2-Amino-N-benzyl-N-(2-benzyl-5,7-dimethyl-1,2,3,4-tetrahydro-naphthalen-1-yl)-acetamide

N-Benzyl-N-(2-benzyl-5,7-dimethyl-1,2,3,4-tetrahydro-naphthalen-1-yl)-2-(1,3-dioxo-1,3-dihydro-isoindol-2-yl)-acetamide (167 mg) is treated as above to afford 81 mg 2-Amino-N-benzyl-N-(2-benzyl-5,7-dimethyl-1,2,3,4-tetrahydro-naphthalen-1-yl)-acetamide, 63%

### 2-Amino-N-benzyl-N-(2-benzyl-6,7-dimethoxy-1,2,3,4-tetrahydro-naphthalen-1-yl)-acetamide

N-Benzyl-N-(2-benzyl-6,7-dimethoxy-1,2,3,4-tetrahydro-naphthalen-1-yl)-2-(1,3-dioxo-1,3-dihydro-isoindol-2-yl)- acetamide (328 mg) is treated as above to afford 127 mg 2-Amino-N-benzyl-N-(2-benzyl-6,7-dimethoxy-1,2,3,4-tetrahydro-naphthalen-1-yl)-acetamide, 50%

### 2-Amino-N-benzyl-N-[2-(4-methyl-benzyl)-1,2,3,4-tetrahydro-naphthalen-1-yl]-acetamide

N-Benzyl-2-(1,3-dioxo-1,3-dihydro-isoindol-2-yl)-N-[2-(4-methyl-benzyl)-1,2,3,4-tetrahydro-naphthalen-1-yl]-acetamide (407 mg) is treated as above to afford 222 mg 2-Amino-N-benzyl-N-[2-(4-methyl-benzyl)-1,2,3,4-tetrahydro-naphthalen-1-yl]-acetamide, 73%.

### 2-Amino-N-benzyl-N-[2-(3-methyl-benzyl)-1,2,3,4-tetrahydro-naphthalen-1-yl]-acetamide

N-Benzyl-2-(1,3-dioxo-1,3-dihydro-isoindol-2-yl)N-[2-(3-methyl-benzyl)-1,2,3,4-tetrahydro-naphthalen-1-yl]-acetamide (423 mg) is treated as above to afford 188 mg 2-Amino-N-benzyl-N-[2-(3-methyl-benzyl)-1,2,3,4-tetrahydro-naphthalen-1-yl]-acetamide, 59%.

### 2-Amino-N-benzyl-N-[2-(3-methoxy-benzyl)-1,2,3,4-tetrahydro-naphthalen-1-yl]-acetamide

N-Benzyl-2-(1,3-dioxo-1,3-dihydro-isoindol-2-yl)-N-[2-(3-methoxy-benzyl)-1,2,3,4-tetrahydro-naphthalen-1-yl]-acetamide (408 mg) is treated as above to afford 193 mg 2-Amino-N-benzyl-N-[2-(3-methoxy-benzyl)-1,2,3,4-tetrahydro-naphthalen-1-yl]-acetamide, 62%.

### 2-Amino-N-benzyl-N-[2-(3,4-dichloro-benzyl)-1,2,3,4-tetrahydro-naphthalen-1-yl]-acetamide

N-Benzyl-N-[2-(3,4-dichloro-benzyl)-1,2,3,4-tetrahydro-naphthalen-1-yl]-2-(1,3-dioxo-1,3-dihydro-isoindol-2-yl)-acetamide (415 mg) is treated as above to afford 194 mg 2-Amino-N-benzyl-N-[2-(3,4-dichloro-benzyl)-1,2,3,4-tetrahydro-naphthalen-1-yl]-acetamide, 60%.

### 2-Amino-N-benzyl-N-[2-(2-methyl-benzyl)-1,2,3,4-tetrahydro-naphthalen-1-yl]-acetamide

N-Benzyl-2-(1,3-dioxo-1,3-dihydro-isoindol-2-yl)-N-[2-(2-methyl-ben?yl)-1,2,3,4-tetrahydro-naphthalen-1-yl]-acetamide (425 mg) is treated as above to afford 234 mg 2-Amino-N-benzyl-N-[2-(2-methyl-benzyl)-1,2,3,4-tetrahydro-naphthalen-1-yl]-acetamide, 73%.

### 2-Amino-N-benzyl-N-[2-(3-fluoro-benzyl)-1,2,3,4-tetrahydro-naphthalen-1-yl]-acetamide

N-Benzyl-2-(1,3-dioxo-1,3-dihydro-isoindol-2-yl)-N-[2-(3-fluoro-benzyl)-1,2,3,4-tetrahydro-naphthalen-1-yl]-acetamide (523 mg) is treated as above to afford 333 mg 2-Amino-N-benzyl-N-[2-(3-fluoro-benzyl)-1,2,3,4-tetrahydro-naphthalen-1-yl]-acetamide, 84%.

### 2-Amino-N-(2-benzo[1,3dioxol-5-ylmethyl-1,2,3,4-tetrahydro-naphthalen-1-yl)-N-benzyl-acetamide

N-(2-Benzo[1,3]dioxol-5-ylmethyl-1,2,3,4-tetrahydro-naphthalen-1-yl)-N-benzyl-2-(1,3-dioxo-1,3-dihydro-isoindol-2-yl)-acetamide (464 mg) is treated as above to afford 269 mg 2-Amino-N-(2-benzo[1,3]dioxol-5-ylmethyl-1,2,3,4-tetrahydro-naphthalen-1-yl)-N-benzyl-acetamide, 76%.

### 2-Amino-N-benzyl-N-[2-(2-chloro-benzyl)-1,2,3,4-tetrahydro-naphthalen-1-yl]-acetamide

N-Benzyl-N-[2-(2-chloro-benzyl)-1,2,3,4-tetrahydro-naphthalen-1-yl]-2-(1,3-dioxo-1,3-dihydro-isoindol-2-yl)-acetamide (351 mg) is treated as above to afford 203 mg 2-Amino-N-benzyl-N-[2-(2-chloro-benzyl)-1,2,3,4-tetrahydro-naphthalen-1-yl]-acetamide, 76%.

### 2-Amino-N-benzyl-N-[2-(4-fluoro-benzyl)-1,2,3,4-tetrahydro-naphthalen-1-yl]-acetamide

N-Benzyl-2-(1,3-dioxo-1,3-dihydro-isoindol-2-yl)-N-[2-(4-fluoro-benzyl)-1,2,3,4-tetrahydro-naphthalen-1-yl]-acetamide (546 mg) is treated as above to afford 267 mg 2-Amino-N-benzyl-N-[2-(4-fluoro-benzyl)-1,2,3,4-tetrahydro-naphthalen-1-yl]-acetamide, 66%.

### 2-Amino-N-benzyl-N-[2-(4-methoxy-benzyl)-1,2,3,4-tetrahydro-naphthalen-1-yl]-acetamide

N-Benzyl-2-(1,3-dioxo-1,3-dihydro-isoindol-2-yl)-N-[2-(4-methoxy-benzyl)-1,2,3,4-tetrahydro-naphthalen-1-yl]-acetamide (455 mg) is treated as above to afford 203 mg 2-Amino-N-benzyl-N-[2-(4-methoxy-benzyl)-1,2,3,4-tetrahydro-naphthalen-i -yl]-acetamide, 59%.

### 2-Amino-N-benzyl-N-[2-(4-chloro-benzyl)-1,2,3,4-tetrahydro-naphthalen-1-yl]-acetamide

N-Benzyl-N-[2-(4-chloro-benzyl)-1,2,3,4-tetrahydro-naphthalen-1-yl]-2-(1,3-dioxo-1,3-dihydro-isoindol-2-yl)-acetamide (426 mg) is treated as above to afford 216 mg 2-Amino-N-benzyl-N-[2-(4-chloro-benzyl)-1,2,3,4-tetrahydro-naphthalen-1-yl]-acetamide, 67%.

### Preparation AJ

### 3-(3-{Benzyl-(3-benzyl-chroman-4-yl)-carbamoyl]-methyl-ureido)-benzoic acid ethyl ester

2-Amino-N-benzyl-N-(3-benzyl-chroman4-4yl)-acetamide (101 mg) in 2.6 ml dichloroethane is treated with 3.0 equiv 3-carboethoxyphenyl isocyanate at ambient temperature 84 hours. The reaction is diluted with 10 ml dichloroethane and treated with 3 equiv aminomethyl resin (Fluka, 1.1 mmole/g) at ambient temperature 24 hours. The resin is filtered off and solvent removed to give 174 mg colorless oil. The oil is applied to a 4 mm prep silica gel plate and developed with 6:4 ethyl acetate:hexanes. The product is removed from the silica gel with THF and methylene chloride. Filtration and evaporation of the filtrate gives 84 mg residue. The residue is applied to another 4 mm prep plate and developed with 2:1 diethyl etherhexanes. The product is removed as before and solvent is evaporated to give 3-(3-{[Benzyl-(3-benzyl-chroman-4-yl)-carbamoyl]-methyl}-ureido)-benzoic acid ethyl ester as a colorless film, 59 mg, 39%.

HPLC*Ret. time 7.73; MS, APCl−(%) 576.2 (M−1, 90%), 219.1 (100%).

Also prepared were:

### 3-(3-([Benzyl-(2-benzyl-6-methoxy-1,2,3,4-tetrahydro-naphthalen-1-yl)-carbamoyl]-methyl}-ureido)-benzoic acid ethyl ester

2-Amino-N-benzyl-N-(2-benzyl-6-methoxy-1,2,3,4-tetrahydro-naphthalen-1-yl)-acetamide (77 mg) is treated as above to afford 3-(3-{[Benzyl-(2-benzyl-6-methoxy-1,2,3,4-tetrahydro-naphthalen-1-yl)-carbamoyl]-methyl}-ureido)-benzoic acid ethyl ester (37 mg) as a colorless film, 34%.

### 3-(3-{[Benzyl-(1,3-diphenyl-propyl)-carbamoyl]-methyl}-ureido)-benzoic acid ethyl ester

2-Amino-N-benzyl-N-(1,3-.diphenyl-propyl)-acetamide (101 mg) is treated as above. The product is purified by prep TLC (1 mm) eluting with 2:1 ether:hexanes to afford 3-(3-{[Benzyl-(1,3-diphenyl-propyl)-carbamoyl]-methyl}-ureido)-benzoic acid ethyl ester, 52 mg, 33%.

### 3-(3-{[Benzyl-(2-benzyl-7-methoxy-1l,2,3,4-tetrahydro-naphthalen-1-yl)-carbamoyl]-methyl}-ureido)-benzoic acid ethyl ester

2-Amino-N-benzyl-N-(2-benzyl-7-methoxy-1,2,3,4-tetrahydro-naphthalen-1-yl)-acetamide (97 mg) in dichloroethane is treated with 0.95 equiv 3-carboethoxyphenyl isocyanate and 2.0 equiv morpholinomethyl resin (2.4 mmole/g) at ambient temperature 18 hours. The reaction is diluted with dichloroethane and treated with 1 equiv aminomethyl resin (Fluka, 1.1 mmole/g) at ambient temperature 5 hours. The resin is filtered off and solvent removed to give yellow oil. The oil is applied to a 1 mm prep silica gel plate and developed with 2:1 diethyl ether:hexanes. The product is removed from the silica gel with THF and methylene chloride. Filtration and evaporation of the filtrate gives 36 mg colorless film, 26%.

### 3-(3-{[Benzyl-(2-benzyl-indan-1-yl)-carbamoyl]-methyl}-ureido)-benzoic acid ethyl ester

2-Amino-N-benzyl-N-(2-benzyl-indan-1-yl)-acetamide (27 mg) is treated as above. The crude products are purified by applying the material in THF to a preconditioned SCX column (1 g, 6 ml) to afford 3-(3-{[Benzyl-(2-benzyl-indan-1-yl)-carbamoyl]-methyl}-ureido)-benzoic acid ethyl ester, 20 mg, 51%.

### 3-(3-{[Benzyl-(2-benzyl-5,7-dimethyl-1,2,3,4-tetrahydro-naphthalen-1-yl)-carbamoyl]-methyl}-ureido)-benzoic acid ethyl ester

2-Amino-N-benzyl-N-(2-benzyl-5,7-dimethyl-1,2,3,4-tetrahydro-naphthalen-1-yl)-acetamide (81 mg) is treated as above. The crude products are purified by applying the material in THF to a preconditioned SCX column (1 g, 6 ml) to afford 3-(3-{[Benzyl-(2-benzyl-5,7-dimethyl-1,2,3,4-tetrahydro-naphthalen-1-yl)-carbamoyl]-methyl}-ureido)-benzoic acid ethyl ester, 77 mg, 63%.

### 3-(3-{[Benzyl-(2-benzyl-6,7-dimethoxy-1,2,3,4-tetrahydro-naphthalen-1-yl)-carbamoyl]-methyl}-ureido)-benzoic acid ethyl ester

2-Amino-N-benzyl-N-(2-benzyl-6,7-dimethoxy-1,2,3,4-tetrahydro-naphthalen-1-yl)-acetamide (127 mg) is treated as above. The crude products are purified by applying the material in THF to a preconditioned SCX column (2 g, 6 ml) to afford 3-(3-{[Benzyl-(2-benzyl-6,7-dimethoxy-1,2,3,4-tetrahydro-naphthalen-1-yl)-carbamoyl]-methyl}-ureido)-benzoic acid ethyl ester, 100 mg, 56%.

### 3-[3-({Benzyl-[2-(4-methyl-benzyl )-1,2,3,4-tetrahydro-naphthalen-1-yl]-carbamoyl}-methyl)-ureido]-benzoic acid ethyl ester

2-Amino-N-benzyl-N-[2-(4-methyl-benzyl)-1,2,3,4-tetrahydro-naphthalen-1-yl]-acetamide (222 mg) is treated as above. In addition to the SCX column. the product was further purified by radial chromatography (4 mm plate), eluting with-1:1 hexane:ethyl acetate to afford 3-[3-({Benzyl-[2-(4-methyl-benzyl)-1,2,3,4-tetrahydro-naphthalen-1-yl]-carbamoyl}-methyl)-ureido]-benzoic acid ethyl ester as a colorless oil, 161 mg, 48%.

### 3-[3-({Benzyl-[2-(2-methyl-benzyl)-1,2,3,4-tetrahydro-naphthalen-1-yl]-carbamoyl}-methyl)-ureido]-benzoic acid ethyl ester

2-Amino-N-benzyl-N-[2-(3-methyl-benzyl)-1,2,3,4-tetrahydro-naphthalen-1-yl]-acetamide (188 mg) is treated and purified as in 3-[3-({Benzyl-[2-(4-methyl-benzyl)-1,2,3,4-tetrahydro-naphthalen-1-yl]-carbamoyl}-methyl)-ureido]-benzoic acid ethyl ester to afford 3-[3-({Benzyl-[2-(2-methyl-benzyl)-1,2,3,4-tetrahydro-naphthalen-1-y]-carbamoyl}-methyl)-ureido]-benzoic acid ethyl ester as a colorless oil, 132 mg, 47%.

### 3-[3-({Benzyl-[2-(3-methoxy-benzyl)-1,2,3,4-tetrahydro-naphthalen-1-yl]-carbamoyl}-methyl)-ureido]-benzoic acid ethyl ester

2-Amino-t-benzyl-N-[2-(3-methoxy-benzyl)-1,2,3,4-tetrahydro-naphthalen-1-yl]-acetamide (193 mg) is treated and purified as in 3-[3-({Benzyl-[2-(4-methyl-benzyl)-1,2,3,4-tetrahydro-naphthalen-1-yl]-carbamoyl-methyl)-ureido]-benzoic acid ethyl ester to afford a colorless oil, 103 mg, 37%.

### 3-[3-({Benzyl-[2-(3,4-dichloro-benzyl)-1,2,3,4-tetrahydro-naphthalen-1-yl]-carbamoyl}-methyl)-ureido]-benzoic acid ethyl ester

2-Amino-N-benzyl-N-[2-(3,4-dichloro-benzyl)-1,2,3,4-tetrahydro-naphthalen-1-yl]-acetamide (194 mg) is treated and purified as in 3-[3-({Benzyl-[2-(4-methyl-benzyl)-1,2,3,4-tetrahydro-naphthalen7 1-yl]-carbamoyl}-methyl)-ureido]-benzoic acid ethyl ester to afford 3-[3-({Benzyl-[2-(3,4-dichloro-benzyl)-1,2,3,4-tetrahydro-naphthalen-1-yl]-carbamoyl}-methyl)-ureido]-benzoic acid ethyl ester as a colorless oil, 104 mg, 37%.

### 3-[3-({Benzyl-[2-(2-methyl-benzyl)-1,2,3,4-tetrahydro-naphthalen-1-yl]-carbamoyl}-methyl)-ureido]-benzoic acid ethyl ester

2-Amino-N-benzyl-N-[2-(2-methyl-benzyl)-1,2,3,4-tetrahydro-naphthalen-1-yl]-acetamide (234 mg) is treated and purified as in 3-[3-({Benzyl-[2-(4-methyl-benzyl)-1,2,3,4-tetrahydro-naphthalen-1-yl]-carbamoyl}-methyl)-(ureido]-benzoic acid ethyl ester to afford 3-[3-(Benzyl-[2-(2-methyl-benzyl)-1,2,3,4-tetrahydro-naphthalen-1-yl]-carbamoyl}-methyl)-ureido]-benzoic acid ethyl ester as a colorless oil, 156 mg, 44%.

### 3-[3-({Benzyl-[2-(3-fluoro-benzyl)-1,2,3,4-tetrahydro-naphthalen-1-yl]-carbamoyl}-methyl)-ureido]-benzoic acid ethyl ester

2-Amino-N-benzyl-N-[2-(3-fluoro-benzyl)-1 2,3,4-tetrahydro-naphthalen-1-yl]-acetamide (333 mg) is treated and purified as in 3-[3-({Benzyl-[2-(4-methyl-benzyl)-1,2,3,4-tetrahydro-naphthalen-1-yl]-carbamoyl}-methyl)-ureido]benzoic acid ethyl ester to afford 3-[3-({Benzyl-[2-(3-fluoro-benzyl)-1,2,3,4-tetrahydro-naphthalen-1-yl]-carbamoyl}-methyl)-ureido]-benzoic acid ethyl ester as a colorless oil, 183 mg, 37%.

### 3-(3-{[(2-Benzo[1,3]dioxol-5-ylmethyl-1,2,3,4-tetrahydro-naphthalen-1-yl)-benzyl-carbamoyl]-methyl}-ureido)-benzoic acid ethyl ester

2-Amino-N-(2-benzo[1,3]dioxol-5-ylmethyl-1,2,3,4-tetrahydro-naphthalen-1-yl)-N-benzyl-acetamide (269 mg) is treated and purified as in 3-[3-({Benzyl-[2-(4-methyl-benzyl)-1,2,3,4-tetrahydro-naphthalen-1-yl]-carbamoyl}-methyl)-ureido]-benzoic acid ethyl ester to afford 3-(3-{[(2-Benzo[1,3]dioxol-5-ylmethyl-1,2,3,4-tetrahydro-naphthalen-1-yl)-benzyl-carbamoyl]-methyl}-ureido)-benzoic acid ethyl ester as a colorless oil, 170 mg, 43%.

### 3-[3-({Benzyl-[2-(2-chloro-benzyl)-1,2,3,4-tetrahydro-naphthalen-1-yl]-carbamoyl}-methyl)-ureido]-benzoic acid ethyl ester

2-Amino-N-benzyl-N-[2-(2-chloro-benzyl)-1,2,3,4-tetrahydro-naphthalen-1-yl]-acetamide (203 mg) is treated and purified as in 3-[3-({Benzyl-[2-(4-methyl-benzyl)-1,2,3,4-tetrahydro-naphthalen-1-yl]-carbamoyl}-methyl)-ureido]-benzoic acid ethyl ester to afford 3-[3-({Benzyl-[2-(2-chloro-benzyl)-1,2,3,4-tetrahydro-naphthalen-1-yl]-carbamoyl}-methyl)-ureido]-benzoic acid ethyl ester as a colorless oil, 119 mg, 39%.

### 3-[3-({Benzyl-[2-(4-fluoro-benzyl)-1,2,3,4-tetrahydro-naphthalen-1-yl]-carbamoyl}-methyl)-ureido]-benzoic acid ethyl ester

2-Amino-N-benzyl-N-[2-(4-fluoro-benzyl)-1,2,3,4-tetrahydro-naphthalen-1-yl]-acetamide (267 mg) is treated and purified as in 3-[3-({Benzyl-[2-(4-methyl-benzyl)-1,2,3,4-tetrahydro-naphthalen-1-yl]-carbamoyl}-methyl)-ureido]-benzoic acid ethyl ester to afford 3-[3-({Benzyl-[2-(4-fluoro-benzyl)-1,2,3,4-tetrahydro-naphthalen-1-yl]-carbamoyl}-methyl)-ureido]-benzoic acid ethyl ester as a colorless oil, 104 mg, 26%.

### 3-[3-({Benzyl-[2-(4-chloro-benzyl)-1,2,3,4-tetrahydro-naphthalen-1-yl]-carbamoyl}-methyl)-ureido]-benzoic acid ethyl ester

2-Amino-N-benzyl-N-[2-(4-chloro-benzyl)-1,2,3,4-tetrahydro-naphthalen-1-yl]-acetamide (216 mg) is treated and purified as in 3-[3-({Benzyl-[2-(4-methyl-benzyl)-1,2,3,4-tetrahydro-naphthalen-1-yl]-carbamoyl}-methyl)-ureido]-benzoic acid.ethyl ester to afford 3-[3-({Benzyl-[2-(4-chloro-benzyl)-i,2,3,4-tetrahydro-naphthalen-1-yl]-carbamoyl}-methyl)-ureido]-benzoic acid ethyl ester as a colorless oil, 117 mg, 37%.

### 3-[3-({Benzyl-[2-(4-methoxy-benzyl)-1,2,3,4-tetrahydro-naphthalen-1-yl]-carbamoyl}-methyl)-ureido]-benzoic acid ethyl ester

2-Amino-N-benzyl-N-[2-(4-methoxy-benzyl)-1,2,3,4-tetrahydro-naphthalen-1-yl]-acetamide (203 mg) is treated and purified as in 3-[3-({Benzyl-[2-(4-methyl-benzyl)-1,2,3,4-tetrahydro-naphthalen-1-yl]-carbamoyl}-methyl)-ureido]-benzoic acid ethyl ester. The resulting residue is dissolved in methylene chloride and divided among two 2 mm prep TLC plates. Elution with 2:1 hexane:ethyl acetate gave 3-[3-({Benzyl-[2-(4-methoxy-benzyl)-1,2,3,4-tetrahydro-naphthalen-1-yl]-carbamoyl}-methyl)-ureido]-benzoic acid ethyl ester as a colorless oil, 63 mg, 21%.

### EXAMPLE 23

### 3-(3-{[Benzyl-(3-benzyl-chroman-4-yl)-carbamoyl]-methyl }-ureido)-benzoic acid

3-(3-{[Benzyl-(3-benzyl-chroman-4-yl)-carbamoyl]-methyl}-ureido)-benzoic acid ethyl ester (59 mg) is suspended in 660 μl methanol and treated with 220 μl 1 M aqueous lithium hydroxide solution at 750° C. one hour. The methanol is evaporated and the residue is suspended in 1 N aqueous hydrochloric acid, extracted 3×1 ml 15% isopropanol/chloroform. The combined extracts are evaporated to a yellow residue, which is dried in vacuo at <40° C. 72 hours to afford the desired product, 50 mg, 92%.

MS, APCl−(%) 548.3 (M−1, 100%); HPLC Ret. time: 6.89 min

Also prepared were:

### 3-(3-{[Benzyl-(2-benzyl-7-methoxy-1,2,3,4-tetrahydro-naphthalen-1-yl)-carbamoyl]-methyl}-ureido)-benzoic acid

3-(3-{[Benzyl-(2-benzyl-7-methoxy-1,2,3 4-tetrahydro-naphthalen-1-yl)-carbamoyl]-methyl}-ureido)-benzoic acid ethyl ester (36 mg) is treated in the same manner as 3-(3-{[Benzyl-(3-benzyl-6chroman-4-yl)-carbamoyl]-methyl}-ureido)-benzoic acid ethyl ester to give 3-(3-{[Benzyl-(2-benzyl-7-methoxy-1,2,3,4-tetrahydro-naphthalen-1-yl)-carbamoyl]-methyl}-ureido)-benzoic acid as a yellow residue, 26 mg, 76%.

MS, APCl−(%) 576 (M−1, 55%), 219.4 (100%); HPLC Ret. time: 7.34 min

### 3-(3-{[Benzyl-(1,3-diphenyl-propyl)-carbamoyl]-methyl}-ureido)-benzoic acid

3-(3-{[Benzyl-(1,3-diphenyl-propyl)-carbamoyl]-methyl}-ureido)-benzoic acid ethyl ester (52 mg) is treated as above to afford 3-(3-{[Benzyl-(1,3-diphenyl-propyl)-carbamoyl]-methyl}-ureido)-benzoic acid as a yellow residue, 29 mg, 59%.

MS, APCl−(%) 520.3 (M−1, 55%), 219.4,(100%); HPLC Ret. time: 6.81 min

### 3-(3-{[Benzyl-(2-benzyl-indan-1-yl)-carbamoyl]-methyl}-ureido)-benzoic acid

3-(3-{[Benzyl-(2-benzyl-indan-1-yl)-carbamoyl]-methyl}-ureido)-benzoic acid ethyl ester (20 mg) is treated as above to give 3-(3-{[Benzyl-(2-benzyl-indan-1-yl)-carbamoyl]-methyl}-ureido)-benzoic acid as a white foam, 19 mg, 100%.

MS, APCl−(%) 532.2 (M−1, 100%); HPLC Ret. time: 6.89 min

### 3-(3-{[Benzyl-(2-benzyl-5,7-dimethyl-1,2,3,4-tetrahydro-naphthalen-1-yl)-carbamoyl]-methyl}-ureido)-benzoic acid

3-(3-{[Benzyl-(2-benzyl-5,7-dimethyl-1,2,3,4-tetrahydro-naphthalen-1-yl)-carbamoyl]-methyl)-ureido)-benzoic acid ethyl ester (72 mg) is treated as above to give 3-(3{[Benzyl-(2-benzyl-5,7-dimethyl-1,2,3,4-tetrahydro-naphthalen-1-yl)-carbamoyl]-methyl}-ureido)-benzoic acid as a white residue, 67 mg, 97%.

MS, APCl−(%) 574.4 (M−1,30%), 610.4 ([M−1] +HCl); HPLC Ret. time: 7.62 min

### 3-(3-{[Benzyl-(2-benzyl-6,7-dimethoxy-1,2,3,4-tetrahydro-naphthalen-1-yl)-carbamoyl]-methyl}-ureido)-benzoic acid

3-(3-{[Benzyl-(2-benzyl-6,7-dimethoxy-1,2,3,4-tetrahydro-naphthalen-1-yl)-carbamoyl]-methyl}-ureido)-benzoic acid ethyl ester (95 mg) is treated as above to give 3-(3-{[Benzyl-(2-benzyl-6,7-dimethoxy-1,2,3,4-tetrahydro-naphthalen-1-yl)-carbamoyl]-methyl}-ureido)-benzoic acid as a white foam, 87 mg, 95%.

MS, APCl−(%) 605.9 (M−1, 90%), 591.6 (100%); HPLC Ret. time: 6.76 min

### 3-[3-({Benzyl-[2-(4-methyl-benzyl)-1,2,3,4-tetrahydro-naphthalen-1-yl]-carbamoyl}-methyl)-ureido]-benzoic acid

3-[3-({Benzyl-[2-(4-methyl-benzyl)-1,2,3,4-tetrahydro-naphthalen-1-yl]-carbamoyl)-methyl)-ureido]-benzoic acid ethyl ester (161 mg) is treated as above to give 3-[3-({Benzyl-[2-(4-methyl-benzyl)-1,2,3,4-tetrahydro-naphthalen-1-yl]-carbamoyl}-methyl)-ureido]-benzoic acid as a white residue, 133 mg, 87%.

MS, APCl−(%) 560.3 (M−1, 20%), 347.0 (100%); HPLC Ret. time: 7.27 min

### 3-[3-({Benzyl-[2-(3-methyl-benzyl)-1,2,3,4-tetrahydro-naphthalen-1-yl]-carbamoyl}-methyl)-ureido]-benzoic acid

3-[3-({Benzyl-[2-(2-methyl-benzyl)-1,2,3,4-tetrahydro-naphthalen-1-yl]-carbamoyl}-methyl)-ureido]-benzoic acid ethyl ester (132 mg) is treated as above to give 3-[3-({Benzyl-[2-(3-methyl-benzyl)-1,2,3,4-tetrahydro-naphthalen-1-yl]-carbamoyl}-methyl)-ureido]-benzoic acid as a white residue, 124 mg, 99%.

MS, APCl−(0%) 560.2 (M−1, 95%), 347.0 (100%); HPLC Ret. time: 7.23 min

### 3-[3-({Benzyl-[2-(3-methoxy-benzyl)-1,2,3,4-tetrahydro-naphthalen-1-yl]-carbamoyl}-methyl)-ureido]-benzoic acid

3-[3-({Benzyl-[2-(3-methoxy-benzyl)-1,2,3,4-tetrahydro-naphthalen-1-yl]-carbamoyl}-methyl)-ureido]-benzoic acid ethyl ester (103 mg) is treated as above to give 3-[3-({Benzyl-[2-(3-methoxy-benzyl)-1,2,3,4-tetrahydro-naphthalen-1-yl]-carbamoyl}-methyl)-ureido]-benzoic acid as a white residue, 99 mg, 99%.

MS, APCl−(%) 576.2 (M−1, 100%); HPLC Ret. time: 6.89 min

### 3-[3-({Benzyl-[2-(3,4-dichloro-benzyl)-1,2,3,4-tetrahydro-naphthalen-1-yl]-carbamoyl}-methyl)-ureido-benzoic acid

3-[3-({Benzyl-[2-(3,4-dichloro-benzyl)-1,2,3,4-tetrahydro-naphthalen-1-yl]-carbamoyl}-methyl)-ureido]-benzoic acid ethyl ester (104 mg) is treated as above to give 3-(3-({Benzyl-[2-(3,4-dichloro-benzyl)-1,2,3,4-tetrahydro-naphthalen-1-yl]-carbamoyl}-methyl)-ureido]-benzoic acid as a white residue, 90 mg, 91%.

MS, APCl−(%) 615 (M−1, 10%), 389 (100%); HPLC Ret. time: 7.64 min

### 3-[3-({Benzyl-[2-(2-methyl-benzyl)-1,2,3,4-tetrahydro-naphthalen-1-yl]-carbamoyl}-methyl)-ureido]-benzoic acid

3-[3-({Benzyl-[2-(2-methyl-benzyl)-1,2,3,4-tetrahydro-naphthalen-1-yl]-carbamoyl}-methyl)-ureido]-benzoic acid ethyl ester (156 mg) is treated as above to give 3-[3-({Benzyl-[2-(2-methyl-benzyl)-1,2,3,4-tetrahydro-naphthalen-1-yl]-carbamoyl}-methyl)-ureido]-benzoic acid as a white residue, 143 mg, 97%.

MS, APCl−(%) 560.2 (M−1, 100%); HPLC Ret. time: 7.19 min

### 3-[3-({Benzyl-[2-(3-fluoro-benzyl)-1,2,3,4-tetrahydro-naphthalen-1-yl]-carbamoyl}-methyl)-ureido]-benzoic acid

3-[3-({Benzyl-[2-(3-fluoro-benzyl)-1,2,3,4-tetrahydro-naphthalen-1-yl]-carbamoyl}-methyl)-ureido]-benzoic acid ethyl ester (183 mg) is treated as above to give 3-[3-({Benzyl-[2-(3-fluoro-benzyl)-1,2,3,4-tetrahydro-naphthalen-1-yl]-carbamoyl}-methyl)-ureido]-benzoic acid as a white residue, 169 mg, 96%.

MS, APCl−(%) 564.1 (M−1, 100%); HPLC Ret. time: 6.99 min

### 3-(3-{[(2-Benzo[1,3]dioxol-5-ylmethyl-1,2,3,4-tetrahydro-naphthalen-1-yl)-benzyl-carbamoyl]-methyl}-ureido)-benzoic acid

3-(3-{[(2-Benzo[1,3]dioxol-5-ylmethyl-1,2,3,4-tetrahydro-naphthalen-1-yl)-benzyl-carbamoyl]-methyl}-ureido)-benzoic acid ethyl ester (170 mg) is treated as above to give 3-(3-{[(2-Benzo[1,3]dioxol-5-ylmethyl-1,2,3,4-tetrahydro-naphthalen-1-yl)-benzyl-carbamoyl]-methyl}-ureido)-benzoic acid as a white residue, 144 mg, 90%.

MS, APCl−(%) 590.0 (M−1, 100%); HPLC Ret. time: 6.78 min

### 3-[3-({Benzyl-[2-(2-chloro-benzyl)-1,2,3,4-tetrahydro-naphthalen-1-yl]-carbamoyl}-methyl)-ureido]-benzoic acid

3-[3-({Benzyl-[2-(2-chloro-benzyl)-1,2,3,4-tetrahydro-naphthalen-1-yl]-carbamoyl}-methyl)-ureido]-benzoic acid ethyl ester (119 mg) is treated as above to give 3-[3-({Benzyl-[2-(2-chloro-benzyl)-1,2,3,4-tetrahydro-naphthalen-1-yl]-carbamoyl}-methyl)-ureido]-benzoic acid as a white residue, 108 mg, 97%.

MS, APCl−(%) 580.1 (M−1, 100%); HPLC Ret. time: 7.22 min

### 3-[3-({Benzyl-[2-(4-fluoro-benzyl)-1,2,3,4-tetrahydro-naphthalen-1-yl]-carbamoyl}-methyl)-ureido]-benzoic acid

3-[3-({Benzyl-[2-(4-fluoro-benzyl)-1,2,3,4-tetrahydro-naphthalen-1-yl]-carbamoyl}-methyl)-ureido]-benzoic acid ethyl ester (104 mg) is treated as above to give 3-[3-({Benzyl-[2-(4-fluoro-benzyl)-1,2,3,4-tetrahydro-naphthalen-1-yl]-carbamoyl}-methyl)-ureido]-benzoic acid as a white residue, 101 mg, 99%.

MS, APCl−(%) 564.3 (M−1, 100%); HPLC Ret. time: 7.03 min

### 3-[3-({Benzyl-[2-(4-methoxy-benzyl)-1,2,3,4-tetrahydro-naphthalen-1-yl]-carbamoyl}-methyl)-ureido]-benzoic acid

3-[3-({Benzyl-[2-(4-methoxy-benzyl)-1,2,3,4-tetrahydro-naphthalen-1-yl]-carbamoyl}-methyl)-ureido]-benzoic acid ethyl ester (63 mg) is treated as above to give 3-[3-({Benzyl-[2-(4-methoxy-benzyl)-1,2,3,4-tetrahydro-naphthalen-1-yl]-carbamoyl}-methyl)-ureido]-benzoic acid as a white residue, 50 mg, 86%.

MS, APCl−(%) 576.1 (M−1, 100%); HPLC Ret. time: 6.87 min

### 3-[3-({Benzyl-[2-(4-chloro-benzyl)-1,2,3,4-tetrahydro-naphthalen-1-yl]-carbamoyl}-methyl)-ureido]-benzoic acid

3-[3-({Benzyl-[2-(4-chloro-benzyl)-1,2,3,4-tetrahydro-naphthalen-1-yl]-carbamoyl}-methyl)-ureido]-be.nzoic acid ethyl ester (117 mg) is treated as above to give 3-[3-({Benzyl-[2-(4-chloro-benzyl)-1,2,3,4-tetrahydro-naphthalen-1-yl]-carbamoyl}-methyl)-ureido]-benzoic acid as a white residue, 84 mg, 76%.

MS, APCl−(%) 580.1 (M−1, 100%/o); HPLC Ret. time: 7.39 min

### 3-(3-{[Benzyl-(2-benzyl-6-methoxy-1,2,3,4-tetrahydro-naphthalen-1-yl)-carbamoyl]-methyl-ureido)-benzoic acid

3-(3-{[Benzyl-(2-benzyl-6-methoxy-1,2,3,4-tetrahydro-naphthalen-1-yl)-carbamoyl]-methyl}-ureido)-benzoic acid ethyl ester (37 mg) is treated as above to afford a yellow residue, which is dissolved in THF and applied to a preconditioned SAX cartridge (500 mg) and eluted with 3×3 ml THF. The product is released with 2 M acetic acid solution in THF, then 2M acetic acid in methanol. Solvent is evaporated to give 3-(3-{[Benzyl-(2-benzyl-6-methoxy-1,2,3,4-tetrahydro-naphthalen-1-yl)-carbamoyl]-methyl}-ureido)-benzoic acid as yellow residue, 31 mg, 88%.

MS, APCl−(%) 576 (M−1, 60%), 132.2 (100%); HPLC Ret. time: 7.15 min

The HPLC system consists of a Waters 717 autosampler, Waters 996 Photodiode Array Detector, and Waters 600 quaternary solvent delivery system, and is controlled by Millennium software. The samples are chromatographed using a linear gradient of 0% to 100% Acetonitrile/0.2 M Ammonium Acetate buffer (pH 4.5) over ten minutes at a flow rate of 3 ml/min. using a Perkin-Elmer Pecosphere 3.3 cm C18 column.

1. A compound of the formula 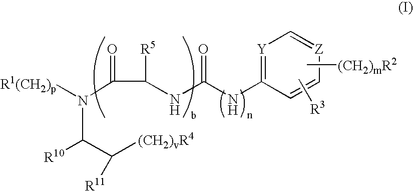
or a pharmaceutically acceptable salt, hydrate or prodrug thereof, wherein:
R¹ and R⁵ are each independently selected from H, C₁-C₁₀ alkyl, C₂-C₁₀ alkenyl, C₂-C₁₀ alkynyl, —(CH₂)ₜ(C₆-C₁₀ aryl), or —(CH₂)ₜ(4 to 10 membered heterocyclic), wherein t is an integer from 0 to 5; said alkyl group optionally including 1 or 2 hetero moieties selected from O, S and —N(R⁶)— with the proviso that two O atoms, two S atoms, or an O and S atom are not attached directly to each other; said aryl and heterocyclic R¹ and R⁵ groups being optionally fused to a C₆-C₁₀ aryl group, a C₅-C₈ saturated cyclic group, or a 4 to 10 membered heterocyclic group; one or two carbon atoms in said 4 to 10 membered heterocyclic group of R¹ and R⁵ being optionally substituted by an oxo (═O) moiety; the —(CH₂)ₜ— moieties of R¹ and R⁵ optionally including a carbon-carbon double or triple bond when t is an integer from two to five; R¹ and R⁵ groups being optionally substituted by one to five R⁶ groups;
each R⁶ is independently selected from C₁-C₁₀ alkyl, C₂-C₁₀ alkenyl, C₂-C₁₀ alkynyl, halo, cyano, nitro, trifluoromethyl, trifluoromethoxy, azido, —OR⁷, —C(O)R⁸, —C(O)OR⁷, —NR⁸C(O)OR⁷, —OC(O)R⁷, —NR⁸SO₂R⁷, —SO₂NR⁷R⁸, —NR⁸C(O)R⁷, —C(O)NR⁷R⁸, —NR⁷R⁸, —S(O)ⱼR⁹ wherein j is an integer ranging from zero to two, —SO₃H, —NR⁷(CR⁸R⁹)ₜOR⁸, —(CH₂)ₜ(C₆-C₁₀ aryl), —SO₂(CH₂)ₜ(C₆-C₁₀ aryl ), —S(CH₂)ₜ(C₆-C₁₀ aryl), —O(CH₂)ₜ(C₆-C₁₀ aryl), —(CH₂)ₜ(4 to 10 membered heterocyclic), and —(CR⁸R⁹)ₘOR⁸ wherein m is an integer from one to five and t is an integer from zero to five; said alkyl group optionally containing one or two hetero moieties selected from O, S and —N(R⁸)— with the proviso that two O atoms, two S atoms, or an O and S atom are not attached directly to each other; aryl and heterocyclic moieties of R⁶ being optionally fused to a C₆-C₁₀ aryl group, a C₅-C₈ saturated cyclic group, or a 4 to 10 membered heterocyclic group; one or two carbon atoms of the heterocyclic moieties of R⁶ being optionally substituted by an oxo (═O) moiety; and the alkyl, aryl and heterocyclic moieties of R⁶ groups being optionally substituted by one to three substituents independently selected from halo, cyano, nitro, trifluoromethyl, trifluoromethoxy, azido, —NR⁸SO₂R⁷, —SO₂NR⁷R⁸, —C(O)R⁷, —C(O)OR⁷, —OC(O)R⁷, —NR⁸C(O)R⁷, —C(O)NR⁷R⁸, —NR⁷R⁸, —(CR⁸R⁹)ₘOR⁸ wherein m is an integer from one to five, —OR⁷ and R⁷;
each R⁷ is independently selected from H, C₁-C₁₀ alkyl, —(CH₂)ₜ(C₆-C₁₀ aryl), and —(CH₂)ₜ(4 to 10 membered heterocyclic), wherein t is an integer from zero to five; said alkyl group optionally including one or two hetero moieties selected from O, S and —N(R⁶)— with the proviso that two O atoms, two S atoms, or an O and S atom are not attached directly to each other; said aryl and heterocyclic R groups being optionally fused to a C₆-C₁₀ aryl group, a C₅-C₈ saturated cyclic group, or a 4 to 10 membered heterocyclic group; the foregoing moieties of R⁷; with the exception of H, being optionally substituted by one to three substituents independently selected from halo, cyano, nitro, trifluoromethyl, trifluoromethoxy, azido, —C(O)R⁸, —C(O)OR⁸, —CO(O)R⁸, —NR⁸C(O)R⁹, —C(O)NR⁸R⁹, —NR⁸R⁹, hydroxy, C₁-C₆ alkyl, and C₁-C₆ alkoxy;
each R⁸ and R⁹ is independently H or C₁-C₆ alkyl;
R² is a group selected from CO₂H, CONHSO₂R¹, CONR¹(CH₂)CO₂H, SO₂H, PO₃H₂, 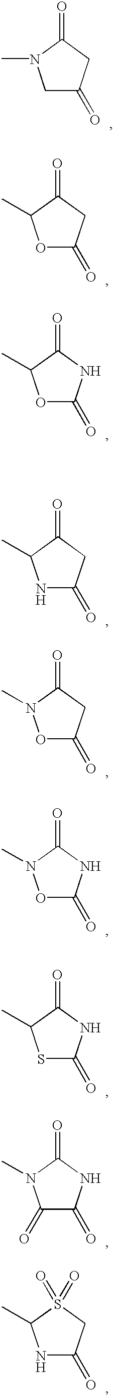 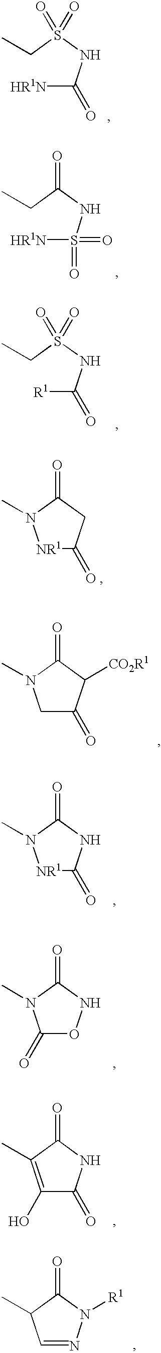 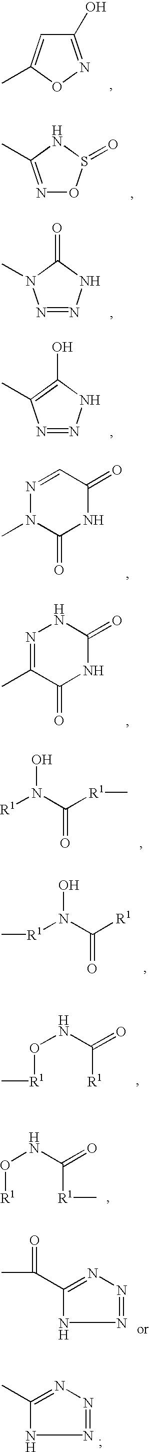
each R³ is independently selected from H and R²;
R⁴ is —(CH₂)ₜ(C₆-C₁₀ aryl), or —(CH₂)ₜ(4 to 10 membered heterocyclic), wherein t is a integer from zero to five; said alkyl group optionally including one or two hetero moieties selected from O, S and —N(R⁶)— with the proviso that two O atoms, two S atoms, or an O and S atom are not attached directly to each other; said aryl and heterocyclic R⁴ groups are optionally fused to a C₆-C₁₀ aryl group, a C₅-C₈ saturated cyclic group, or a 4 to 10 membered heterocyclic group; one or two carbon atoms of the heterocyclic moieties of R⁴ being optionally substituted by an oxo (═O) moiety; the —(CH₂)ₜ— moieties of R⁴ optionally including a carbon-carbon double or triple bond where t is an integer from two to five, R⁴ being optionally substituted by one to five R⁶ groups or methylenedioxy;
R¹⁰ and R¹¹ are each independently R¹, or R¹⁰ and R¹¹, together with the carbons to which R¹⁰ and R¹¹ are attached, optionally form a 4 to 10 membered carbocyclic group optionally substituted by ═O or H(OH) or a 4 to 10 membered heterocyclic group comprising heterocyclic moieties selected from O, N or S optionally substituted with R¹, S, SO or SO₂; said carbocyclic group or heterocyclic group formed by R¹⁰ and R¹¹ being optionally fused to a C₆-C₁₀ aryl group, a C₅-C₈ saturated cyclic group, or a 4 to 10 membered heterocyclic group optionally substituted with one or more substituents selected from halogen, hydroxy, C₁-C₁₀ alkyl, C₁-C₁₀ alkoxy and methylenedioxy;
Y and Z are independently CH, N optionally substituted with R¹, O, S, SO or SO₂;
m is zero or 1;
n is zero or 1;
b is zero or 1;
v is zero or 1 and
p is zero to 6,
with the proviso that said compound of formula I is not 3-(3-{[benzyl-(2-benzyl-1,2,3,4-tetrahydro-naphthalen-1-yl)-carbamoyl]-methyl}-ureido)-benzoic acid or 3-(3-{[2(2-benzyl-1,2,3,4-tetrahydro-naphthalen-1-yl)-isoxalol-5-ylmethyl-carbamoyl]-methyl}-ureido)-benzoic acid.

2. The compound of claim 1, wherein R¹ and R⁵ are each independently selected from H, C₁-C₁₀ alkyl, C₂-C₁₀ alkenyl, C₂-C₁₀ alkynyl, C₆-C₁₀ aryl, or a 4 to 10 membered heterocyclic group, wherein any aromatic carbocyclic or heterocyclic rings are optionally substituted with one or more substituents selected from halogen, hydroxy, C₁-C₆alkoxy, C₁-C₆alkyl, CF₃, CO₂H, CO₂-C₁-C₆alkyl or CN.

3. The compound of claim 1, wherein R² is selected from —CO₂H, —CONHSO₂R¹, —CONR¹(CH₂)CO₂H,

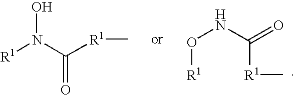

4. The compound of claim 1, wherein R² is selected from meta-substituted benzoic acid and phenylacetic acetic acid.

5. The compound of claim 1, wherein R⁴ is phenyl or a 4 to 10 membered heterocyclic group optionally substituted with one or more substituents selected from halogen, hydroxy, C₁-C₆alkoxy, C₁-C₆alkyl, and methylenedioxy.

6. The compound of claim 1, wherein Y and Z are each independently selected from CH and N.

7. A compound of claim 1 of the following formula: 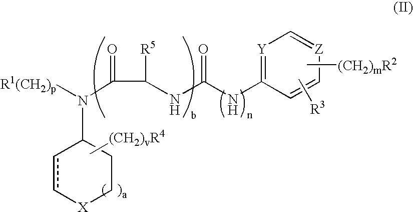
wherein R¹, R², R³, R⁴, R⁵, b, m, n, p, v, Y and Z are as defined for formula (1),( )ₐ means (CH₂)ₐ, X is CHR¹, O, NR¹, S, SO or SO₂, a is zero, 1 or 2; and the dotted line indicates optional fusion to a C₆-C₁₀ aryl group, a C₅-C₈ saturated cyclic group, or a 4 to 10 membered heterocyclic group, each optionally substituted with one or more substituents selected from halogen, hydroxy, C₁-C₁₀ alkyl, C₁-C₁₀ alkoxy and methylenedioxy.

8. A compound of claim 7 selected from:
trans-3-(3-{[Benzyl-(2-benzyl-1,2,3,4-tetrahydro-naphthalen-1-yl)-carbamoyl]-methyl}-ureido)-benzoic acid;
trans-2-[3-(3-Benzenesulfonylaminocarbonyl-phenyl)-ureido]-N-benzyl-N-(2-benzyl-1,2,3,4-tetrahydro-naphthalen-1-yl)-acetamide;
trans-3-(3-{[Benzyl-(2-benzyl-1,2,3,4-tetrahydro-naphthalen-1-yl)-carbamoyl]-methyl}-ureido)-N-(1H-tetrazol-5-yl)-benzamide;
trans-N-{[Benzyl-(2-benzyl-1,2,3,4-tetrahydro-naphthalen-1-yl)-carbamoyl]-methyl}-3-(1H-tetrazol-5-yl)-benzamide;
trans-[3-(3-{[Benzyl-(2-benzyl-1,2,3,4-tetrahydro-naphthalen-1-yl)-carbamoyl]-methyl}-ureido)-phenyl]-acetic acid;
cis-[3-(3-{[Benzyl-(2-benzyl-1,2,3,4-tetrahydro-naphthalen-1-yl)-carbamoyl]-methyl}-ureido)-phenyl]-acetic acid;
cis-3-(3-{[(2-Benzyl-1,2,3,4-tetrahydro-naphthalen-1-yl)-furan-2-ylmethyl-carbamoyl]-methyl}-ureido)-benzoic acid;
trans-3-(3[(2-Benzyl-1,2,3,4-tetrahydro-naphthalen-1-yl)-furan-2-ylmethyl-carbamoyl]-methyl}-ureido)-benzoic acid;
trans-3-(3-([(2-Benzyl-1,2,3,4-tetrahydro-naphthalen-1-yl)-isoxazol-5-ylmethyl-carbamoyl]-methyl}-ureido)-benzoic acid;
trans-3-(3[(2-Benzyl-1 ,2 ,3,4-tetrahydro-naphthalen-1-yl)-(4-fluoro-benzyl)-carbamoyl]-methyl}-ureido)-benzoic acid;
trans-3-(3-{[(2-Benzyl-1,2,3,4-tetrahydro-naphthalen-1-yl)-(2-methoxy-benzyl)-carbamoyl]-methyl}-ureido)-benzoic acid;
trans-3-(3-{[(2-Benzyl-1,2,3,4-tetrahydro-naphthalen-1-yl)-pyridin-2-ylmethyl-carbamoyl]-methyl}-ureido)-benzoic acid;
trans-3-(3-{[(2-Benzyl-1,2,3,4-tetrahydro-naphthalen-1-yl)-thiophen-2-ylmethyl-carbamoyl]-methyl}-ureido)-benzoic acid;
trans-3-(3-{[Benzyl-(2-benzyl-cyclohexyl)-carbamoyl]-methyl}-ureido)-benzoic acid
trans-3-(3-{[(2-Benzyl-1,2,3,4-tetrahydro-naphthalen-1-yl)-pyridin-4-ylmethyl-carbamoyl]-methyl}-ureido)-benzoic acid;
trans-3-(3-{[(2-Benzyl-1,2,3,4-tetrahydro-naphthalen-1-yl)-ethyl-carbamoyl]-methyl}-ureido)-benzoic acid;
trans-3-(3-{[(2-Benzyl-1,2 ,3,4-tetrahydro-naphthalen-1-yl)-thiazol-2-ylmethyl-carbamoyl]-methyl}-ureido)-benzoic acid;
trans-3-[3-({Benzyl-[2-(2-methoxy-benzyl)-1,2,3,4-tetrahydronaphthalen-1-yl]-carbamoyl}-methyl)-ureido]-benzoic acid;
trans-3-[3-({Benzyl-[2-(2-fluoro-benzyl)-1,2,3,4-tetrahydro-naphthalen-1-yl]-carbamoyl}-methyl)-ureido]-benzoic acid;
trans-6-({[Benzyl-(2-benzyl-cyclohexyl)-carbamoyl]-methyl)-carbamoyl)-pyrimidine-4-carboxylic acid;
trans-4-(3-{1-[Benzyl-(2-benzyl-1,2,3,4-tetrahydro-naphthalen-1-yl)-carbamoyl]-2-phenyl-ethyl}-ureido)-phthalic acid; trans-4-[3-Benzyl-3-(2-benzyl-1,2,3,4-tetrahydro-naphthalen-1-yl)-ureido]-phthalic acid;
3-(3-{[Benzyl-(3-benzyl-chroman-4-yl)-carbamoyl]-methyl}-ureido)-benzoic acid;
3-(3-{[Benzyl-(2-benzyl-7-methoxy-1,2,3,4-tetrahydro-naphthalen-1-yl)-carbamoyl]-methyl}-ureido)-benzoic acid;
3-(3-{[Benzyl-(1,3-diphenyl-propyl )-carbamoyl]-methyl}-ureido)-benzoic acid;
3-(3{[Benzyl-(2-benzyl-indan-1-yl)-carbamoyl]-methyl}-ureido)-benzoic acid;
3-(3-{[Benzyl-(2-benzyl-5,7-dimethyl-1,2,3,4-tetrahydro-naphthalen-1-yl)-carbamoyl]-methyl}-ureido)-benzoic acid;
3-(3-{[Benzyl-(2-benzyl-6,7-dimethoxy-1,2 ,3,4-tetrahydro-naphthalen-1-yl)-carbamoyl]-methyl}-ureido)-benzoic acid;
3-[3-({Benzyl-[2-(4-methyl-benzyl)-1,2,3,4-tetrahydro-naphthalen-1-yl]-carbamoyl}-methyl)-ureido]-benzoic acid;
3-[3-({Benzyl-[2-(3-methyl-benzyl)-1,2,3,4-tetrahydro-naphthalen-1-yl]-carbamoyl}-methyl)-ureido]-benzoic acid;
3-[3-({Benzyl-[2-(3-methoxy-benzyl)-1,2,3,4-tetrahydro-naphthalen-1-yl]-carbamoyl}-methyl)-ureido]-benzoic acid;
3-[3-({Benzyl-[2-(3,4-dichloro-benzyl)-1,2,3,4-tetrahydro-naphthalen-1-yl]-carbamoyl}-methyl)-ureido]-benzoic acid;
3-[3-({Benzyl-[2-(2-methyl-benzyl )-1,2,3,4-tetrahydro-naphthalen-1-yl]-carbamoyl}-methyl)-ureido]-benzoic acid;
3-[3-({Benzyl-[2-(3-fluoro-benzyl)-1,2 ,3,4-tetrahydro-naphthalen-1-yl]-carbamoyl}-methyl)-ureido]-benzoic acid;
3-(3-{[(2-Benzo[1,3]dioxol-5-ylmethyl-1,2,3,4-tetrahydro-naphthalen-1-yl)-benzyl-carbamoyl]-methyl}-ureido)-benzoic acid;
3-[3-({Benzyl-[2-(2-chloro-benzyl)-1,2,3,4-tetrahydro-naphthalen-1-yl]-carbamoyl}-methyl)-ureido]-benzoic acid;
3-[3-({Benzyl-[2-(4-fluoro-benzyl)-1,2,3,4-tetrahydro-naphthalen-1-yl]-carbamoyl}-methyl)-ureido]-benzoic acid;
3-[3-({Benzyl-[2-(4-methoxy-benzyl)-1,2,3,4-tetrahydro-naphthalen-1-yl]-carbamoyl}-methyl)-ureido]-benzoic acid;
3-[3-({Benzyl-[2-(4-chloro-benzyl)-1,2,3,4-tetrahydro-naphthalen-1-yl]-carbamoyl}-methyl)-ureido]-benzoic acid;
3-(3{[Benzyl-(2-benzyl-6-methoxy-1,2,3,4-tetrahydro-naphthalen-1-yl)-carbamoyl]-methyl}-ureido)-benzoic acid
3-(3-{[2-benzyl-1,2,3,4-tetrahydro-naphthalen-1-yl)-furan-2-ylmethyl-carbamoyl]-methyl}-ureido)-benzoic acid;
N-{[benzyl-(2-benzyl-1,2,3,4-tetrahydro-napthalen-1-yl)-carbamoyl]-methyl}-3-(1H-tetrazol-5-yl)-benzamide; and
2-[3-(3-benzenesulfonylaminocarbonyl-phenyl)-ureido]-N-benzyl-N-(2-benzyl-1,2,3,4-tetrahydro-naphthalen-1-yl)-acetamide.

9. A compound of claim 7 selected from:
3-[3-({Benzyl-[2-(3-fluoro-benzyl)-1,2,3,4-tetrahydro-naphthalen-1-yl]-carbamoyl}-methyl)-ureido]-benzoic acid;
3-(3-{[(2-Benzo[1,3]dioxol-5-ylmethyl-1,2,3,4-tetrahydro-naphthalen-1-yl)-benzyl-carbamoyl]-methyl}-ureido)-benzoic acid;
3-[3-({Benzyl-[2-(2-chloro-benzyl)-1,2,3,4-tetrahydro-naphthalen-1-yl]-carbamoyl}-methyl)-ureido]-benzoic acid;
3-[3-({Benzyl-[2-(4-fluoro-benzyl)-1,2,3,4-tetrahydro-naphthalen-1-yl]-carbamoyl}-methyl)-ureido]-benzoic acid;
3-[3-({Benzyl-[2-(4-methoxy-benzyl)-1,2,3 ,4-tetrahydro-naphthalen-1-yl]-carbamoyl}-methyl)-ureido]-benzoic acid;
3-[3-({Benzyl-[2-(4-chloro-benzyl)-1,2,3,4-tetrahydro-naphthalen-1-yl]-carbamoyl}-methyl)-ureido]-benzoic acid;
3-(3-{[Benzyl-(2-benzyl-6-methoxy-1,2,3,4-tetrahydro-naphthalen-1-yl)-carbamoyl]-methyl}-ureido)-benzoic acid
3-(3-{[2-benzyl-1,2,3,4-tetrahydro-naphthalen-1-yl)-furan-2-ylmethyl-carbamoyl]-methyl}-ureido)-benzoic acid;
N-{[benzyl-(2-benzyl-1,2,3,4-tetrahydro-napthalen-1-yl)-carbamoyl]-methyl}-3-(1H-tetrazol-5-yl)-benzamide; and
2-[3-(3-benzenesulfonylaminocarbonyl-phenyl)-ureido]-N-benzyl-N -(2-benzyl-1,2,3,4-tetrahydro-naphthalen-1yl)-acetamide.

10. A pharmaceutical composition for the treatment of a hyperproliferative disorder in a mammal which comprises a therapeutically effective amount of a compound according to claim 1, and a pharmaceutically acceptable carrier.

11. A pharmaceutical composition for the treatment of pancreatitis or kidney disease in a mammal which comprises a therapeutically effective amount of a compound according to claim 1, and a pharmaceutically acceptable carrier.

12. A pharmaceutical composition for the prevention of blastocyte implantation in a mammal which comprises a therapeutically effective amount of a compound according to claim 1, and a pharmaceutically acceptable carrier.

13. A pharmaceutical composition for treating a disease related to vasculogenesis or angiogenesis in a mammal which comprises a therapeutically effective amount of a compound according to claim 1, and a pharmaceutically acceptable carrier.

14. A method of treating a hyperproliferative disorder in a mammal in need of such treatment which comprises administering to said mammal a therapeutically effective amount of the compound according to claim 1, or 3-(3-{[benzyl-(2-benzyl-1,2,3,4-tetrahydro-naphthalen-1-yl)-carbamoyl]-methyl)-ureido)-benzoic acid or 3-(3-{[2(2-benzyl-1,2,3,4-tetrahydro-naphthalen-1-yl)-isoxalol-5-ylmethyl-carbamoyl]-methyl}-ureido)-benzoic acid.

15. A method of the treating a hyperproliferative disorder in a mammal which comprises administering to a mammal in need of such treatment a therapeutically effective amount of a compound according to claim 1, 3-(3-{[benzyl-(2-benzyl-1,2,3,4-tetrahydro-naphthalen-1yl)-carbamoyl]-methyl}-ureido)-benzoic acid or 3-(3-([2(2-benzyl-1,2,3,4-tetrahydro-naphthalen-1-yl)-isoxalol-5-ylmethyl-carbamoyl]-methyl}-ureido)-benzoic acid, in combination with an anti-tumor agent selected from the group consisting of mitotic inhibitors, alkylating agents, anti-metabolites, intercalating antibiotics, growth factor inhibitors, cell cycle inhibitors, enzymes, topoisomerase inhibitors, biological response modifiers, anti-hormones, and anti-androgens.

16. A method of treating pancreatitis or kidney disease in a mammal which comprises administering to a mammal in need of such treatment a therapeutically effective amount of a compound according to claim 1 or 3-(3-{[benzyl-(2-benzyl-1,2,3,4-tetrahydro-naphthalen-1-yl)-carbamoyl]-methyl}-ureido)-benzoic acid or 3-(3-{[2(2-benzyl-1,2,3,4-tetrahydro-naphthalen-1-yl)-isoxalol-5-ylmethyl-carbamoyl]-methyl}-ureido)-benzoic acid.

17. A method of preventing blastocyte implantation in a mammal which comprises administering to said mammal a therapeutically effective amount of a compound according to claim 1, 3-(3-([benzyl-(2-benzyl-1,2,3,4-tetrahydro-naphthalen-1-yl)-carbamoyl]-methyl}-ureido)-benzoic acid or 3-(3-{[2(2-benzyl-1,2,3,4-tetrahydro-naphthalen-1-yl)-isoxalol-5-ylmethyl-carbamoyl]-methyl}-ureido)-benzoic acid.

18. A method of treating diseases related to vasculogenesis or angiogenesis in a mammal which comprises administering to a mammal in need of such treatment an effective amount of a compound according to claim 1 or 3-(3-{[benzyl-(2-benzyl-1,2,3,4-tetrahydro-naphthalen-1-yl)-carbamoyl]-methyl}-ureido)-benzoic acid or 3-(3-{[2(2-benzyl-1,2,3,4-tetrahydro-naphthalen-1-yl)-isoxalol-5-ylmethyl-carbamoyl]-methyl}-ureido)-benzoic acid.

19. A process for forming a compound of formula III 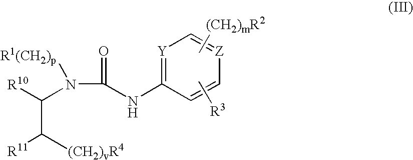
which comprises reacting a compound of formula VIII 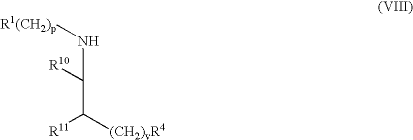
with a compound of formula IX 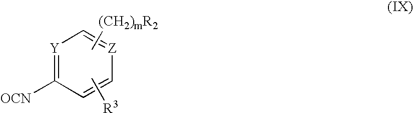
wherein R¹, R², R³, R⁴, R¹⁰, R¹¹, m, p and v are as defined in claim 1

20. A process for forming a compound of formula IV 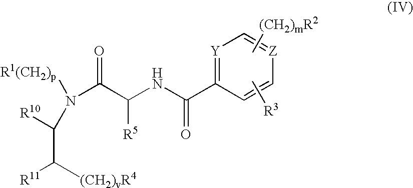
which comprises reacting a compound of formula XII 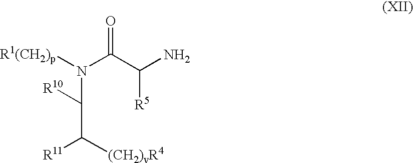
with a compound of formula XIII 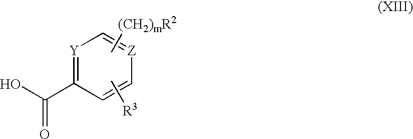
wherein R¹, R², R³, R⁴, R⁵, R¹⁰, R¹¹, m, p and v are as defined in claim 1.

21. A process for forming a compound of formula V 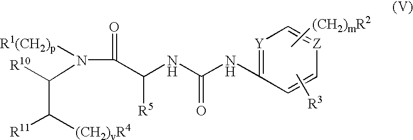
which comprises reacting a compound of formula XII 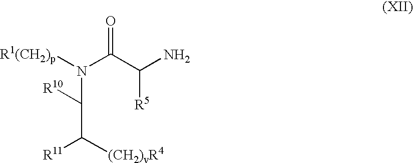
with a compound of formula IX 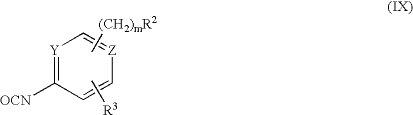
wherein R¹, R², R³, R⁴, R⁵, R¹⁰, R¹¹, m, p, and v are as defined in claim 1.
